(19) United States
(12) Patent Application Publication
(10) Pub. No.: US 2026/0146228 A1
(43) Pub. Date: May 28, 2026

(54) COMPOSITIONS AND METHODS FOR INDUCING OOCYTE MATURATION

(71) Applicant: Gameto, Inc., Austin, TX (US)

(72) Inventors: Christian KRAMME, Austin, TX (US); Dina Radenkovic, Austin, TX (US); Martin Varsavsky, Austin, TX (US); Klaus Wiemer, Austin, TX (US)

(21) Appl. No.: 19/181,929

(22) Filed: Apr. 17, 2025

Related U.S. Application Data
(63) Continuation of application No. 18/875,590.
Application No. 18/875,590 is a continuation of application No. 17/846,725, filed on Jun. 22, 2022, now Pat. No. 11,802,268.
Application No. 17/846,725 is a continuation of application No. 17/846,845, filed on Jun. 22, 2022, now abandoned.
(60) Provisional application No. 63/492,210, filed on Mar. 24, 2023.

(51) Int. Cl. C12N 5/075; A61K 31/194; A61K 38/24; C12N 5/00; G16B 40/00

(52) U.S. Cl. CPC C12N 5/0609; A61K 31/194; A61K 38/24; C12N 5/0062; G16B 40/00; C12N 2502/243; C12N 2506/45; C12N 2513/00

(57) ABSTRACT

Featured are methods, compositions, and apparatuses for the in vitro maturation of oocytes. In particular, the disclosure features methods of inducing oocyte maturation in vitro, by co-culturing a female subject's oocytes with an ex vivo composition containing a plurality of ovarian support cells (e.g., granulosa cells). Additional methods for administering follicular triggering agents and retrieving oocytes from the female subject are provided. Such methods, compositions, and apparatuses are particularly useful for assisted reproduction technology (ART) procedures.

### TECHNICAL FIELD

This disclosure relates to the field of in vitro oocyte maturation.

### BACKGROUND

One in ten women struggle with infertility, requiring assisted reproductive technology (ART) such as in vitro fertilization (IVF). Challenges remain with maintaining oocyte health in culture, resulting in low oocyte quality and subsequently poor embryo quality. Furthermore, oocytes that are developmentally immature are traditionally discarded, constricting the available oocyte pool for IVF. In vitro maturation (IVM) holds the promise to mature oocytes in vitro after egg extraction, allowing for utilization of all retrieved eggs. Current methods for IVM are inefficient, using follicle-stimulating hormone (FSH) spike-in to the culture media, showing 5-40% maturation of immature eggs. Even worse, this method results in many unhealthy eggs, with an embryo viability rate under 17%, far lower than for standard IVF. Thus, there remains a need in the field for promoting oocyte maturation for a female subject undergoing an ART procedure.

### SUMMARY OF THE INVENTION

In one aspect, the disclosure features a method of inducing oocyte maturation in vitro, the method including co-culturing one or more oocytes that have previously been retrieved from a human subject with a population of ovarian support cells.

In a further aspect, the disclosure features a method of preparing one or more oocytes that have previously been retrieved from a human subject for use in an assisted reproduction technology (ART) procedure, the method including co-culturing the one or more oocytes with a population of ovarian support cells.

In a further aspect, the disclosure features a method of producing a mature oocyte for use in an ART procedure, the method including co-culturing one or more oocytes that have previously been retrieved from a human subject with a population of ovarian support cells.

In some embodiments, prior to retrieval of the one or more oocytes from the subject, the subject is administered one or more follicular triggering agents during a follicular triggering period.

In some embodiments, prior to retrieval of the one or more oocytes from the subject, the subject is not administered a follicular triggering agent during a follicular triggering period.

In some embodiments, the follicular triggering period has a duration of no greater than 8 days. In some embodiments, the follicular triggering period has a duration of no greater than 7 days. In some embodiments, the follicular triggering period has a duration of no greater than 6 days. In some embodiments, the follicular triggering period has a duration of no greater than 5 days. In some embodiments, the follicular triggering period has a duration of no greater than 4 days. In some embodiments, the follicular triggering period has a duration of no greater than 3 days. In some embodiments, the follicular triggering period has a duration of no greater than 2 days. In some embodiments, the follicular triggering period has a duration of no greater than 1 day. In some embodiments, the follicular triggering period has a duration of from 1 day to 8 days. In some embodiments, the follicular triggering period has a duration of from 1 day to 7 days. In some embodiments, the follicular triggering period has a duration of from 1 day to 6 days. In some embodiments, the follicular triggering period has a duration of from 1 day to 5 days. In some embodiments, the follicular triggering period has a duration of from 1 day to 4 days. In some embodiments, the follicular triggering period has a duration of from 1 day to 3 days. In some embodiments, the follicular triggering period has a duration of from 2 days to 8 days. In some embodiments, the follicular triggering period has a duration of from 2 days to 7 days. In some embodiments, the follicular triggering period has a duration of from 2 days to 6 days. In some embodiments, the follicular triggering period has a duration of from 2 days to 5 days. In some embodiments, the follicular triggering period has a duration of from 2 days to 4 days. In some embodiments, the follicular triggering period has a duration of from 3 days to 8 days. In some embodiments, the follicular triggering period has a duration of from 3 days to 7 days. In some embodiments, the follicular triggering period has a duration of from 3 days to 6 days. In some embodiments, the follicular triggering period has a duration of from 3 days to 5 days.

In some embodiments, the one or more follicular triggering agents include follicle stimulating hormone (FSH), clomiphene citrate, and/or human chorionic gonadotropin (hCG). In some embodiments, the one or more follicular triggering agents include FSH.

In some embodiments, the FSH is administered to the subject in one or more doses per day. In some embodiments, the FSH is administered to the subject once daily.

In some embodiments, the FSH is administered to the subject in an amount of from about 100 international units (IU) to about 1,000 IU per day. In some embodiments, the FSH is administered to the subject in an amount of from about 200 IU to about 800 IU per day. In some embodiments, the FSH is administered to the subject in an amount of from about 300 IU to about 700 IU per day. In some embodiments, the FSH is administered to the subject in an amount of from about 300 IU to about 600 IU per day, from about 300 IU to about 500 IU per day, or from about 300 IU to about 400 IU per day.

In some embodiments, the duration of FSH administration is equal to the duration of the follicular triggering period. In some embodiments, the duration of FSH administration is less than the duration of the follicular triggering period. In some embodiments, the duration of FSH administration is 1, 2, 3, 4, or 5 days during the follicular triggering period, optionally wherein the FSH is administered to the subject in an amount of about 200 IU per day for 1, 2, 3, 4, or 5 days during the follicular triggering period, optionally wherein the FSH is administered to the subject in an amount of about 200 IU per day for 3 days during the follicular triggering period.

In some embodiments, the one or more follicular triggering agents include clomiphene citrate. In some embodiments, the clomiphene citrate is administered to the subject in one or more doses per day.

In some embodiments, the clomiphene citrate is administered to the subject once daily.

In some embodiments, the clomiphene citrate is administered to the subject in an amount of from about 50 mg to about 100 mg per day. In some embodiments, the clomiphene citrate is administered to the subject in an amount of about 50 mg per day.

In some embodiments, the duration of clomiphene citrate administration is equal to the duration of the follicular triggering period. In some embodiments, the duration of clomiphene citrate administration is less than the duration of the follicular triggering period. In some embodiments, the duration of clomiphene citrate administration is 1, 2, 3, 4, or 5 days during the follicular triggering period.

In some embodiments, the one or more follicular triggering agents include hCG. In some embodiments, the hCG is administered to the subject in one or more doses per day. In some embodiments, the hCG is administered to the subject in 1, 2, or 3 doses during the follicular triggering period.

In some embodiments, the hCG is administered to the subject in an amount of from about 200 μg to about 700 μg per dose. In some embodiments, the hCG is administered to the subject in an amount of from about 200 μg to about 500 μg per dose, from about 300 μg to about 600 μg per dose, from about 400 μg to about 700 μg per dose, from about 200 μg to about 300 μg per dose, from about 300 μg to about 400 μg per dose, from about 400 μg to about 500 μg per dose, from about 500 μg to about 600 μg per dose, or from about 600 μg to about 700 μg per dose. In some embodiments, the hCG is administered to the subject in an amount of about 500 μg per dose. In some embodiments, the hCG is administered to the subject in an amount of from about 2,500 IU to about 10,000 IU per dose.

In some embodiments, the subject is one that has completed oral contraceptive treatment within 28 days of commencement of the follicular triggering period. In some embodiments, the follicular triggering period commences at least 5 days after cessation of the contraceptive treatment.

In some embodiments, the subject has not undergone oral contraceptive treatment within 28 days of commencement of the follicular triggering period.

In some embodiments, the follicular triggering period commences on day 2 of the subject's menstrual cycle.

In some embodiments, the contraceptive treatment includes administration to the subject of a gonadotropin-releasing hormone (GnRH) agonist.

In some embodiments, the subject has been determined to exhibit a follicle size of from about 6 mm to about 8 mm prior to commencement of the follicular triggering period. In some embodiments, the subject has been determined to exhibit a follicle size of from about 6 mm to about 8 mm prior to administration of a final follicular triggering agent. In an embodiment, the follicle size is determined using a scoring metric (e.g., following ultrasound imaging or other follicle size determination method known in the art).

In some embodiments, a biological sample isolated from the subject prior to retrieval of the one or more oocytes has been determined to have an anti-Müllerian hormone (AMH) concentration of from about 0.1 ng/ml to about 1 ng/ml, or from about 1 ng/ml to about 6 ng/ml.

In some embodiments, the sample has been determined to have an AMH concentration of from about 1 ng/ml to about 6 ng/ml, optionally wherein the sample has been determined to have an AMH concentration of from about 2.5 ng/ml to about 3.0 ng/ml. In some embodiments, the sample has been determined to have an AMH concentration of from about 2 ng/ml to about 5 ng/ml. In some embodiments, the sample has been determined to have an AMH concentration of from about 2.5 ng/ml to about 3.0 ng/ml. In some embodiments, a biological sample isolated from the subject prior to retrieval of the one or more oocytes has been determined to have an AMH concentration of at least 1 ng/ml. In some embodiments, a biological sample isolated from the subject prior to retrieval of the one or more oocytes has been determined to have an AMH concentration of no greater than 6 ng/ml. In some embodiments, the biological sample isolated from the subject prior to retrieval of the one or more oocytes has been determined to have an AMH concentration of from about 0.1 ng/ml to about 1 ng/ml. In some embodiments, the sample is a blood sample.

In some embodiments, the subject is from 18 years old to 48 years old at the time of retrieval of the one or more oocytes. In some embodiments, the subject is from 25 years old to 45 years old at the time of retrieval of the one or more oocytes. In some embodiments, the subject is less than 35 years old at the time of retrieval of the one or more oocytes. In some embodiments, the subject is greater than 35 years old at the time of retrieval of the one or more oocytes.

In some embodiments, prior to retrieval of the one or more oocytes from the subject, the subject has been determined to exhibit a follicle size of from about 6 mm to about 14 mm. In some embodiments, prior to retrieval of the one or more oocytes from the subject, the subject has been determined to exhibit a follicle size of from about 8 mm to about 12 mm. In some embodiments, prior to retrieval of the one or more oocytes from the subject, the subject has been determined to exhibit a follicle size of from about 8 mm to about 9 mm. In some embodiments, prior to retrieval of the one or more oocytes from the subject, the subject has been determined to exhibit a follicle size of no greater than 14 mm.

In some embodiments, the follicle size has been assessed by way of ultrasound image analysis.

In some embodiments, a total of 20 oocytes or less are retrieved from the subject. In some embodiments, 15 oocytes or less are retrieved from the subject. In some embodiments, 10 oocytes or less are retrieved from the subject. In some embodiments, 9 oocytes or less are retrieved from the subject. In some embodiments, 8 oocytes or less are retrieved from the subject. In some embodiments, 7 oocytes or less are retrieved from the subject. In some embodiments, 6 oocytes or less are retrieved from the subject. In some embodiments, 5 oocytes or less are retrieved from the subject. In some embodiments, a plurality of oocytes are retrieved from the subject.

In some embodiments, 10% to 100% of the oocytes retrieved from the subject are germinal vesicle (GV)-stage or meiosis I (MI)-stage oocytes. In some embodiments, 20% to 100% of the oocytes retrieved from the subject are GV-stage or MI-stage oocytes. In some embodiments, 30% to 100% of the oocytes retrieved from the subject are GV-stage or MI-stage oocytes. In some embodiments, 40% to 100% of the oocytes retrieved from the subject are GV-stage or MI-stage oocytes. In some embodiments, 50% to 100% of the oocytes retrieved from the subject are GV-stage or MI-stage oocytes. In some embodiments, 60% to 100% of the oocytes retrieved from the subject are GV-stage or MI-stage oocytes. In some embodiments, 70% to 100% of the oocytes retrieved from the subject are GV-stage or MI-stage oocytes. In some embodiments, 80% to 100% of the oocytes retrieved from the subject are GV-stage or MI-stage oocytes. In some embodiments, 90% to 100% of the oocytes retrieved from the subject are GV-stage or MI-stage oocytes. In some embodiments, 100% of the oocytes retrieved from the subject are GV-stage or MI-stage oocytes.

In some embodiments, the population of ovarian support cells includes ovarian granulosa cells and/or ovarian stroma cells, optionally wherein the ovarian granulosa cells are forkhead box protein L2 (FOXL2)-positive and/or wherein the ovarian stroma cells are nuclear receptor subfamily 2 group F member 2 (NR2F2)-positive.

In some embodiments, the population of ovarian support cells includes from about 50,000 to about 100,000 ovarian support cells. In some embodiments, the population of ovarian support cells includes from about 50,000 to about 60,000 ovarian support cells, from about 60,000 to about 70,000 ovarian support cells, from about 70,000 to about 80,000 ovarian support cells, from about 80,000 to about 90,000 ovarian support cells, from about 90,000 to about 100,000 ovarian support cells, or from about 100,000 to about 150,000, optionally wherein the population of ovarian support cells includes about 125,000 ovarian support cells. In some embodiments, the population of ovarian support cells includes about 50,000 ovarian support cells, about 55,000 ovarian support cells, about 60,000 ovarian support cells, about 65,000 ovarian support cells, about 70,000 ovarian support cells, about 75,000 ovarian support cells, about 80,000 ovarian support cells, about 85,000 ovarian support cells, about 90,000 ovarian support cells, about 95,000 ovarian support cells, about 100,000 ovarian support cells, about 105,000 ovarian support cells, about 110,000 ovarian support cells, about 115,000 ovarian support cells, about 120,000 ovarian support cells, about 125,000 ovarian support cells, about 130,000 ovarian support cells, about 135,000 ovarian support cells, about 140,000 ovarian support cells, about 145,000 ovarian support cells, or about 150,000 ovarian support cells.

In some embodiments, the population of ovarian support cells includes of mixture of cell types (e.g., granulosa cells, stroma cells, among other possible cell types). In some embodiments, the population of ovarian support cells includes a mixture of cells such that the mixture comprises a 1:1 distribution of cell types. In some embodiments, the population of ovarian support cells includes a mixture of cell types such that the mixture comprises an unequal distribution of cell types (e.g., 2:1, 3:1, 4:1, 5:1, among other possible population distributions).

In some embodiments, the ovarian support cells include steroidogenic granulosa cells. In some embodiments, the steroidogenic granulosa cells produce estradiol.

In some embodiments, the ovarian support cells are obtained by differentiation of a population of induced pluripotent stem cells (iPSCs).

In some embodiments, the ovarian support cells are obtained by modifying the iPSCs to express one or more transcription factors selected from FOXL2, NR5A1, GATA4, RUNX1, and RUNX2. In some embodiments, the ovarian support cells are obtained by modifying the iPSCs to express two or more of FOXL2, NR5A1, GATA4, RUNX1, and RUNX2. In some embodiments, the ovarian support cells are obtained by modifying the iPSCs to express three or more of FOXL2, NR5A1, GATA4, RUNX1, and RUNX2. In some embodiments, the ovarian support cells are obtained by modifying the iPSCs to express four or more of FOXL2, NR5A1, GATA4, RUNX1, and RUNX2. In some embodiments, the ovarian support cells are obtained by modifying the iPSCs to express all five of FOXL2, NR5A1, GATA4, RUNX1, and RUNX2.

In some embodiments, the ovarian support cells are cryopreserved and thawed prior to the co-culturing with the one or more oocytes. In some embodiments, the ovarian support cells are thawed from about 24 hours to about 120 hours prior to the co-culturing with the one or more oocytes. In some embodiments, the ovarian support cells are thawed from about 24 hours to about 48 hours, from about 48 hours to about 72 hours, from about 72 hours to about 96 hours, or from about 96 hours to about 120 hours prior to the co-culturing with the one or more oocytes. In some embodiments, the ovarian support cells are thawed from about 24 hours to about 36 hours, from about 30 hours to about 40 hours, from about 36 hours to about 48 hours, from about 48 hours to about 56 hours, from about 56 hours to about 72 hours, from about 72 hours to about 84 hours, from about 80 hours to about 96 hours, from about 90 hours to about 100 hours, from about 96 hours to about 108 hours, or from about 108 hours to about 120 hours prior to the co-culturing with the one or more oocytes.

In some embodiments, the one or more oocytes are co-cultured with the population of ovarian support cells for from about 12 hours to about 120 hours. In some embodiments, the one or more oocytes are co-cultured with the population of ovarian support cells for from about 12 hours to about 24 hours, from about 12 hours to about 36 hours, from about 24 hours to about 48 hours, from about 36 hours to about 60 hours, from about 54 hours to about 72 hours, from about 68 hours to about 96 hours, or from about 96 hours to about 120 hours. In some embodiments, the one or more oocytes are co-cultured with the population of ovarian support cells for about 12 hours, about 14 hours, about 16 hours, about 18 hours, about 20 hours, about 22 hours, about 24 hours, about 26 hours, about 28 hours, about 30 hours, about 32 hours, about 34 hours, about 36 hours, about 38 hours, about 40 hours, about 42 hours, about 44 hours, about 46 hours, about 48 hours, about 50 hours, about 52 hours, about 54 hours, about 56 hours, about 58 hours, about 60 hours, about 62 hours, about 64 hours, about 66 hours, about 68 hours, about 70 hours, about 72 hours, about 74 hours, about 76 hours, about 78 hours, about 80 hours, about 82 hours, about 84 hours, about 86 hours, about 88 hours, about 90 hours, about 92 hours, about 94 hours, about 96 hours, about 98 hours, about 100 hours, about 102 hours, about 104 hours, about 106 hours, about 108 hours, about 110 hours, about 112 hours, about 114 hours, about 116 hours, about 118 hours, or about 120 hours.

In some embodiments, the co-culturing is conducted in an adherent co-culture system. In some embodiments, the co-culturing is conducted in a suspension co-culture system.

In some embodiments, prior to and/or after the co-culturing, the one or more oocytes are evaluated for a parameter selected from the group consisting of total oocyte score, GV-stage to MII-stage oocyte maturation rate, GV-stage to MI-stage oocyte maturation rate, MI-stage to MII-stage oocyte maturation rate, average oocyte shape, average oocyte size, average ooplasm quality, average perivitelline space (PVS) quality, average zona pellucida (ZP) quality, and average polar body quality. In some embodiments, the one or more oocytes are denuded following the co-culturing.

In some embodiments, the method further including isolating one or more meiosis II (MII)-stage oocytes from the mixture produced by co-culturing the one or more oocytes retrieved from the subject with the population of ovarian support cells.

In some embodiments, the subject is undergoing an autologous ART procedure, and wherein the method further includes contacting each of the one or more MII-stage oocytes with a mature sperm cell.

In some embodiments, the one or more MII-stage oocytes are cryopreserved and thawed prior to the contacting. In some embodiments, the one or more MII-stage oocytes are not cryopreserved and thawed prior to the contacting.

In some embodiments, the contacting includes in vitro fertilization (IVF) of the one or more MII-stage oocytes. In some embodiments, the contacting includes intracytoplasmic sperm injection (ICSI) into the one or more MII-stage oocytes.

In some embodiments, the contacting results in formation of an embryo. In some embodiments, the embryo is transferred to the uterus of the subject. In some embodiments, the embryo is transferred to the uterus of the subject about 3 days following the contacting of the one or more MII-stage oocytes with a mature sperm cell. In some embodiments, the embryo is transferred to the uterus of the subject about 5 days following the contacting of the one or more MII-stage oocytes with a mature sperm cell. In some embodiments, the embryo transferred to the uterus of the subject is a blastocyst-stage embryo.

In some embodiments, the method results in the formation of a plurality of embryos having a viability rate that exceeds 20% (e.g., a viability rate of 25%, 30%, 35%, 40%, 45%, 50%, 55%, 60%, 65%, 70%, 75%, 80%, 85%, 90%, 95%, 99%, or more).

In a further aspect, the disclosure features a method of producing a mature oocyte for use in an ART procedure, the method including: (a) administering to a human subject one or more follicular triggering agents during a follicular triggering period; (b) retrieving one or more oocytes from the subject following the follicular triggering period; and (c) culturing the one or more oocytes with a population of ovarian support cells, thereby producing one or more mature oocytes.

In a further aspect, the disclosure features a method of promoting oocyte maturation for a subject undergoing an ART procedure and that has previously been administered one or more follicular triggering agents during a follicular triggering period, the method including: (a) retrieving one or more oocytes from the subject; (b) culturing the one or more oocytes with a population of ovarian support cells, thereby producing one or more mature oocytes; and (c) isolating the one or more mature oocytes.

In some embodiments, the follicular triggering period has a duration of no greater than 8 days. In some embodiments, the follicular triggering period has a duration of no greater than 7 days. In some embodiments, the follicular triggering period has a duration of no greater than 6 days. In some embodiments, the follicular triggering period has a duration of no greater than 5 days. In some embodiments, the follicular triggering period has a duration of no greater than 4 days. In some embodiments, the follicular triggering period has a duration of no greater than 3 days. In some embodiments, the follicular triggering period has a duration of no greater than 2 days. In some embodiments, the follicular triggering period has a duration of no greater than 1 day. In some embodiments, the follicular triggering period has a duration of from 1 day to 8 days. In some embodiments, the follicular triggering period has a duration of from 1 day to 7 days. In some embodiments, the follicular triggering period has a duration of from 1 day to 6 days. In some embodiments, the follicular triggering period has a duration of from 1 day to 5 days. In some embodiments, the follicular triggering period has a duration of from 1 day to 4 days. In some embodiments, the follicular triggering period has a duration of from 1 day to 3 days. In some embodiments, the follicular triggering period has a duration of from 2 days to 8 days. In some embodiments, the follicular triggering period has a duration of from 2 days to 7 days. In some embodiments, the follicular triggering period has a duration of from 2 days to 6 days. In some embodiments, the follicular triggering period has a duration of from 2 days to 5 days. In some embodiments, the follicular triggering period has a duration of from 2 days to 4 days. In some embodiments, the follicular triggering period has a duration of from 3 days to 8 days. In some embodiments, the follicular triggering period has a duration of from 3 days to 7 days. In some embodiments, the follicular triggering period has a duration of from 3 days to 6 days. In some embodiments, the follicular triggering period has a duration of from 3 days to 5 days.

In some embodiments, the one or more follicular triggering agents include FSH, clomiphene citrate, and/or hCG. In some embodiments, the one or more follicular triggering agents include FSH.

In some embodiments, the FSH is administered to the subject in one or more doses per day. In some embodiments, the FSH is administered to the subject once daily.

In some embodiments, the FSH is administered to the subject in an amount of from about 100 IU to about 1,000 IU per day. In some embodiments, the FSH is administered to the subject in an amount of from about 200 IU to about 800 IU per day. In some embodiments, the FSH is administered to the subject in an amount of from about 300 IU to about 700 IU per day. In some embodiments, the FSH is administered to the subject in an amount of from about 300 IU to about 600 IU per day, from about 300 IU to about 500 IU per day, or from about 300 IU to about 400 IU per day.

In some embodiments, the duration of FSH administration is equal to the duration of the follicular triggering period. In some embodiments, the duration of FSH administration is less than the duration of the follicular triggering period. In some embodiments, the duration of FSH administration is 1, 2, 3, 4, or 5 days during the follicular triggering period, optionally wherein the FSH is administered to the subject in an amount of about 200 IU per day for 1, 2, 3, 4, or 5 days during the follicular triggering period, optionally wherein the FSH is administered to the subject in an amount of about 200 IU per day for 3 days during the follicular triggering period.

In some embodiments, the one or more follicular triggering agents include clomiphene citrate.

In some embodiments, the clomiphene citrate is administered to the subject in one or more doses per day. In some embodiments, the clomiphene citrate is administered to the subject once daily.

In some embodiments, the clomiphene citrate is administered to the subject in an amount of from about 50 mg to about 100 mg per day. In some embodiments, the clomiphene citrate is administered to the subject in an amount of about 50 mg per day.

In some embodiments, the duration of clomiphene citrate administration is equal to the duration of the follicular triggering period. In some embodiments, the duration of clomiphene citrate administration is less than the duration of the follicular triggering period. In some embodiments, the duration of clomiphene citrate administration is 1, 2, 3, 4, or 5 days during the follicular triggering period.

In some embodiments, the one or more follicular triggering agents include hCG. In some embodiments, the hCG is administered to the subject in one or more doses per day. In some embodiments, the hCG is administered to the subject in 1, 2, or 3 doses during the follicular triggering period.

In some embodiments, the hCG is administered to the subject in an amount of from about 200 μg to about 700 μg per dose. In some embodiments, the hCG is administered to the subject in an amount of from about 200 μg to about 500 μg per dose, from about 300 μg to about 600 μg per dose, from about 400 μg to about 700 μg per dose, from about 200 μg to about 300 μg per dose, from about 300 μg to about 400 μg per dose, from about 400 μg to about 500 μg per dose, from about 500 μg to about 600 μg per dose, or from about 600 μg to about 700 μg per dose. In some embodiments, the hCG is administered to the subject in an amount of about 500 μg per dose. In some embodiments, the hCG is administered to the subject in an amount of from about 2,500 IU to about 10,000 IU per dose.

In some embodiments, the subject is one that has completed oral contraceptive treatment within 28 days of commencement of the follicular triggering period. In some embodiments, the follicular triggering period commences at least 5 days after cessation of the contraceptive treatment.

In some embodiments, the subject has not undergone oral contraceptive treatment within 28 days of commencement of the follicular triggering period.

In some embodiments, the follicular triggering period commences on day 2 of the subject's menstrual cycle.

In some embodiments, the contraceptive treatment includes administration to the subject of a GnRH agonist.

In some embodiments, the subject has been determined to exhibit a follicle size of from about 6 mm to about 8 mm prior to commencement of the follicular triggering period.

In some embodiments, the subject has been determined to exhibit a follicle size of from about 6 mm to about 8 mm prior to administration of a final follicular triggering agent.

In some embodiments, a biological sample isolated from the subject prior to retrieval of the one or more oocytes has been determined to have an AMH concentration of from about 1 ng/ml to about 6 ng/ml. In some embodiments, the sample has been determined to have an AMH concentration of from about 2 ng/ml to about 5 ng/ml. In some embodiments, the sample has been determined to have an AMH concentration of from about 2.5 ng/ml to about 3.0 ng/ml. In some embodiments, a biological sample isolated from the subject prior to retrieval of the one or more oocytes has been determined to have an AMH concentration of at least 1 ng/ml. In some embodiments, a biological sample isolated from the subject prior to retrieval of the one or more oocytes has been determined to have an AMH concentration of no greater than 6 ng/ml.

In some embodiments, the sample is a blood sample.

In some embodiments, the subject is from 18 years old to 48 years old at the time of retrieval of the one or more oocytes. In some embodiments, the subject is from 20 years old to 45 years old. In some embodiments, the subject is from 25 years old to 45 years old at the time of retrieval of the one or more oocytes. In some embodiments, the subject is less than 35 years old at the time of retrieval of the one or more oocytes. In some embodiments, the subject is greater than 35 years old at the time of retrieval of the one or more oocytes.

In some embodiments, prior to retrieval of the one or more oocytes from the subject, the subject has been determined to exhibit a follicle size of from about 6 mm to about 14 mm. In some embodiments, prior to retrieval of the one or more oocytes from the subject, the subject has been determined to exhibit a follicle size of from about 8 mm to about 12 mm. In some embodiments, prior to retrieval of the one or more oocytes from the subject, the subject has been determined to exhibit a follicle size of from about 8 mm to about 9 mm. In some embodiments, prior to retrieval of the one or more oocytes from the subject, the subject has been determined to exhibit a follicle size of no greater than 14 mm.

In some embodiments, the follicle size has been assessed by way of ultrasound image analysis.

In some embodiments, a total of 20 oocytes or less are retrieved from the subject. In some embodiments, 15 oocytes or less are retrieved from the subject. In some embodiments, 10 oocytes or less are retrieved from the subject. In some embodiments, 9 oocytes or less are retrieved from the subject. In some embodiments, 8 oocytes or less are retrieved from the subject. In some embodiments, 7 oocytes or less are retrieved from the subject. In some embodiments, 6 oocytes or less are retrieved from the subject. In some embodiments, 5 oocytes or less are retrieved from the subject. In some embodiments, a plurality of oocytes are retrieved from the subject.

In some embodiments, 10% to 100% of the oocytes retrieved from the subject are GV-stage or MI-stage oocytes. In some embodiments, 20% to 100% of the oocytes retrieved from the subject are GV-stage or MI-stage oocytes. In some embodiments, 30% to 100% of the oocytes retrieved from the subject are GV-stage or MI-stage oocytes. In some embodiments, 40% to 100% of the oocytes retrieved from the subject are GV-stage or MI-stage oocytes. In some embodiments, 50% to 100% of the oocytes retrieved from the subject are GV-stage or MI-stage oocytes. In some embodiments, 60% to 100% of the oocytes retrieved from the subject are GV-stage or MI-stage oocytes. In some embodiments, 70% to 100% of the oocytes retrieved from the subject are GV-stage or MI-stage oocytes. In some embodiments, 80% to 100% of the oocytes retrieved from the subject are GV-stage or MI-stage oocytes. In some embodiments, 90% to 100% of the oocytes retrieved from the subject are GV-stage or MI-stage oocytes. In some embodiments, 100% of the oocytes retrieved from the subject are GV-stage or MI-stage oocytes.

In some embodiments, the population of ovarian support cells includes ovarian granulosa cells and/or ovarian stroma cells, optionally wherein the ovarian granulosa cells are FOXL2-positive and/or wherein the ovarian stroma cells are NR2F2-positive. In some embodiments, the population of ovarian support cells includes of mixture of ovarian granulosa cells and ovarian stroma cells. In some embodiments, the population of ovarian support cells includes a mixture of cells such that the mixture comprises approximately a 1:1 distribution of ovarian granulosa cells and ovarian stroma cells, with or without one or more additional cell types in the population. In some embodiments, the population of ovarian support cells includes a mixture of cell types such that the mixture comprises distribution of cell types in which one or more cell type is more abundant compared to another cell type (e.g., a relative distribution of 2:1, 3:1, 4:1, 5:1, among other possible population distributions). In some embodiments, the population of ovarian support cells includes a mixture of ovarian granulosa cells and ovarian stroma cells such that one cell type is more abundant in the mixture (e.g., 90% ovarian granulosa cells and 10% ovarian stroma cells, 80% ovarian granulosa cells and 20% ovarian stroma cells, 70% ovarian granulosa cells and 30% ovarian stroma cells, 60% ovarian granulosa cells and 40% ovarian stroma cells, 40% ovarian granulosa cells and 60% ovarian stroma cells, 30% ovarian granulosa cells and 70% ovarian stroma cells, 20% ovarian granulosa cells and 80% ovarian stroma cells, or 10% ovarian granulosa cells and 90% ovarian stroma cells, among other possible distributions). In some embodiments, the population of ovarian support cells includes a mixture of ovarian granulosa cells and ovarian stroma cells in combination with one or more additional cell types.

In some embodiments, the population of ovarian support cells includes ovarian granulosa cells.

In some embodiments, the population of ovarian support cells includes from about 50,000 to about 100,000 ovarian support cells. In some embodiments, the population of ovarian support cells includes from about 50,000 to about 60,000 ovarian support cells, from about 60,000 to about 70,000 ovarian support cells, from about 70,000 to about 80,000 ovarian support cells, from about 80,000 to about 90,000 ovarian support cells, or from about 90,000 to about 100,000 ovarian support cells. In some embodiments, the population of ovarian support cells includes about 50,000 ovarian support cells, about 55,000 ovarian support cells, about 60,000 ovarian support cells, about 65,000 ovarian support cells, about 70,000 ovarian support cells, about 75,000 ovarian support cells, about 80,000 ovarian support cells, about 85,000 ovarian support cells, about 90,000 ovarian support cells, about 95,000 ovarian support cells, or about 100,000 ovarian support cells.

In some embodiments, the ovarian support cells include steroidogenic granulosa cells. In some embodiments, the steroidogenic granulosa cells produce estradiol.

In some embodiments, the ovarian support cells are obtained by differentiation of a population of iPSCs.

In some embodiments, the ovarian support cells are obtained by modifying the iPSCs to express one or more transcription factors selected from FOXL2, NR5A1, GATA4, RUNX1, and RUNX2. In some embodiments, the ovarian support cells are obtained by modifying the iPSCs to express two or more of FOXL2, NR5A1, GATA4, RUNX1, and RUNX2. In some embodiments, the ovarian support cells are obtained by modifying the iPSCs to express three or more of FOXL2, NR5A1, GATA4, RUNX1, and RUNX2. In some embodiments, the ovarian support cells are obtained by modifying the iPSCs to express four or more of FOXL2, NR5A1, GATA4, RUNX1, and RUNX2. In some embodiments, the ovarian support cells are obtained by modifying the iPSCs to express all five of FOXL2, NR5A1, GATA4, RUNX1, and RUNX2.

In some embodiments, the ovarian support cells are cryopreserved and thawed prior to the co-culturing with the one or more oocytes. In some embodiments, the ovarian support cells are thawed from about 24 hours to about 120 hours prior to the co-culturing with the one or more oocytes. In some embodiments, the ovarian support cells are thawed from about 24 hours to about 48 hours, from about 48 hours to about 72 hours, from about 72 hours to about 96 hours, or from about 96 hours to about 120 hours prior to the co-culturing with the one or more oocytes. In some embodiments, the ovarian support cells are thawed from about 24 hours to about 36 hours, from about 30 hours to about 40 hours, from about 36 hours to about 48 hours, from about 48 hours to about 56 hours, from about 56 hours to about 72 hours, from about 72 hours to about 84 hours, from about 80 hours to about 96 hours, from about 90 hours to about 100 hours, from about 96 hours to about 108 hours, or from about 108 hours to about 120 hours prior to the co-culturing with the one or more oocytes.

In some embodiments, the one or more oocytes are co-cultured with the population of ovarian support cells for from about 12 hours to about 120 hours. In some embodiments, the one or more oocytes are co-cultured with the population of ovarian support cells for from about 12 hours to about 24 hours, from about 12 hours to about 36 hours, from about 24 hours to about 48 hours, from about 36 hours to about 60 hours, from about 54 hours to about 72 hours, from about 68 hours to about 96 hours, or from about 96 hours to about 120 hours. In some embodiments, the one or more oocytes are co-cultured with the population of ovarian support cells for about 12 hours, about 14 hours, about 16 hours, about 18 hours, about 20 hours, about 22 hours, about 24 hours, about 26 hours, about 28 hours, about 30 hours, about 32 hours, about 34 hours, about 36 hours, about 38 hours, about 40 hours, about 42 hours, about 44 hours, about 46 hours, about 48 hours, about 50 hours, about 52 hours, about 54 hours, about 56 hours, about 58 hours, about 60 hours, about 62 hours, about 64 hours, about 66 hours, about 68 hours, about 70 hours, about 72 hours, about 74 hours, about 76 hours, about 78 hours, about 80 hours, about 82 hours, about 84 hours, about 86 hours, about 88 hours, about 90 hours, about 92 hours, about 94 hours, about 96 hours, about 98 hours, about 100 hours, about 102 hours, about 104 hours, about 106 hours, about 108 hours, about 110 hours, about 112 hours, about 114 hours, about 116 hours, about 118 hours, or about 120 hours.

In some embodiments, the co-culturing is conducted in an adherent co-culture system. In some embodiments, the co-culturing is conducted in a suspension co-culture system.

In some embodiments, prior to and/or after the co-culturing, the one or more oocytes are evaluated for a parameter selected from the group consisting of total oocyte score, GV-stage to MII-stage oocyte maturation rate, GV-stage to MI-stage oocyte maturation rate, MI-stage to MII-stage oocyte maturation rate, average oocyte shape, average oocyte size, average ooplasm quality, average perivitelline space (PVS) quality, average zona pellucida (ZP) quality, and average polar body quality.

In some embodiments, the one or more oocytes are denuded following the co-culturing.

In some embodiments, the method further includes isolating one or more MII-stage oocytes from the mixture produced by co-culturing the one or more oocytes retrieved from the subject with the population of ovarian support cells.

In some embodiments, the subject is undergoing an autologous ART procedure, and wherein the method further includes contacting each of the one or more MII-stage oocytes with a mature sperm cell.

In some embodiments, the one or more MII-stage oocytes are cryopreserved and thawed prior to the contacting. In some embodiments, the one or more MII-stage oocytes are not cryopreserved and thawed prior to the contacting.

In some embodiments, the contacting includes IVF of the one or more MII-stage oocytes. In some embodiments, the contacting includes ICSI into the one or more MII-stage oocytes.

In some embodiments, the contacting results in formation of an embryo. In some embodiments, the embryo is transferred to the uterus of the subject. In some embodiments, the embryo is transferred to the uterus of the subject about 3 days following the contacting of the one or more MII-stage oocytes with a mature sperm cell. In some embodiments, the embryo is transferred to the uterus of the subject about 5 days following the contacting of the one or more MII-stage oocytes with a mature sperm cell. In some embodiments, the embryo transferred to the uterus of the subject is a blastocyst-stage embryo.

In some embodiments, the method results in the formation of a plurality of embryos having a viability rate that exceeds 20% (e.g., a viability rate of 25%, 30%, 35%, 40%, 45%, 50%, 55%, 60%, 65%, 70%, 75%, 80%, 85%, 90%, 95%, 99%, or more).

In a further aspect, the disclosure features an ex vivo composition including a population of ovarian support cells and one or more diluents or excipients.

In some embodiments, the population of ovarian support cells includes from about 50,000 to about 100,000 ovarian support cells. In some embodiments, the population of ovarian support cells includes from about 50,000 to about 60,000 ovarian support cells, from about 60,000 to about 70,000 ovarian support cells, from about 70,000 to about 80,000 ovarian support cells, from about 80,000 to about 90,000 ovarian support cells, or from about 90,000 to about 100,000 ovarian support cells. In some embodiments, the population of ovarian support cells includes about 50,000 ovarian support cells, about 55,000 ovarian support cells, about 60,000 ovarian support cells, about 65,000 ovarian support cells, about 70,000 ovarian support cells, about 75,000 ovarian support cells, about 80,000 ovarian support cells, about 85,000 ovarian support cells, about 90,000 ovarian support cells, about 95,000 ovarian support cells, or about 100,000 ovarian support cells.

In some embodiments, the ovarian support cells include steroidogenic granulosa cells. In some embodiments, the steroidogenic granulosa cells produce estradiol.

In some embodiments, the ovarian support cells are obtained by differentiation of a population of iPSCs.

In some embodiments, the ovarian support cells are obtained by modifying the iPSCs to express one or more transcription factors selected from FOXL2, NR5A1, GATA4, RUNX1, and RUNX2. In some embodiments, the ovarian support cells are obtained by modifying the iPSCs to express two or more of FOXL2, NR5A1, GATA4, RUNX1, and RUNX2. In some embodiments, the ovarian support cells are obtained by modifying the iPSCs to express three or more of FOXL2, NR5A1, GATA4, RUNX1, and RUNX2. In some embodiments, the ovarian support cells are obtained by modifying the iPSCs to express four or more of FOXL2, NR5A1, GATA4, RUNX1, and RUNX2. In some embodiments, the ovarian support cells are obtained by modifying the iPSCs to express all five of FOXL2, NR5A1, GATA4, RUNX1, and RUNX2.

In some embodiments, the ovarian support cells are cryopreserved.

In a further aspect, the disclosure features a cell culture medium including a population of ovarian support cells.

In some embodiments, the population of ovarian support cells includes from about 50,000 to about 100,000 ovarian support cells. In some embodiments, the population of ovarian support cells includes from about 50,000 to about 60,000 ovarian support cells, from about 60,000 to about 70,000 ovarian support cells, from about 70,000 to about 80,000 ovarian support cells, from about 80,000 to about 90,000 ovarian support cells, or from about 90,000 to about 100,000 ovarian support cells. In some embodiments, the population of ovarian support cells includes about 50,000 ovarian support cells, about 55,000 ovarian support cells, about 60,000 ovarian support cells, about 65,000 ovarian support cells, about 70,000 ovarian support cells, about 75,000 ovarian support cells, about 80,000 ovarian support cells, about 85,000 ovarian support cells, about 90,000 ovarian support cells, about 95,000 ovarian support cells, or about 100,000 ovarian support cells.

In some embodiments, the ovarian support cells include steroidogenic granulosa cells. In some embodiments, the steroidogenic granulosa cells produce estradiol.

In some embodiments, the ovarian support cells are obtained by differentiation of a population of iPSCs.

In some embodiments, the ovarian support cells are obtained by modifying the iPSCs to express one or more transcription factors selected from FOXL2, NR5A1, GATA4, RUNX1, and RUNX2. In some embodiments, the ovarian support cells are obtained by modifying the iPSCs to express two or more of FOXL2, NR5A1, GATA4, RUNX1, and RUNX2. In some embodiments, the ovarian support cells are obtained by modifying the iPSCs to express three or more of FOXL2, NR5A1, GATA4, RUNX1, and RUNX2. In some embodiments, the ovarian support cells are obtained by modifying the iPSCs to express four or more of FOXL2, NR5A1, GATA4, RUNX1, and RUNX2. In some embodiments, the ovarian support cells are obtained by modifying the iPSCs to express all five of FOXL2, NR5A1, GATA4, RUNX1, and RUNX2.

In some embodiments, the cell culture medium is cryopreserved.

In a further aspect, the disclosure features the composition of any one of the foregoing aspects or the cell culture medium of any one of the foregoing aspects for use in performing the method of any one of the foregoing aspects.

In a further aspect, the disclosure features a kit including the composition of any one of the foregoing aspects and a package insert, wherein the package insert instructs a user of the kit to co-culture the population of ovarian support cells with one or more oocytes in accordance with the method of any one of the foregoing aspects.

In a further aspect, the disclosure features a kit including the cell culture medium of any one of the foregoing aspects and a package insert, wherein the package insert instructs a user of the kit to co-culture the population of ovarian support cells with one or more oocytes in accordance with the method of any one of the foregoing aspects.

### BRIEF DESCRIPTION OF THE DRAWINGS

The accompanying drawings are included to illustrate embodiments of the disclosure and further an understanding of its implementations.

FIG. 1A is a block diagram of an embodiment and an apparatus for aiding in human oocyte maturation in vitro.

FIG. 1B is an exemplary embodiment of an apparatus 100 for aiding in oocyte rescue in vitro post stimulation.

FIG. 2A is a block diagram of exemplary embodiment of a machine learning module.

FIG. 2B is an exemplary table illustrating training data for training a machine learning model.

FIG. 2C is an exemplary table illustrating additional training data for training a machine learning model.

FIG. 3A is an exemplary flow-chart of a mini stimulation protocol.

FIG. 3B is an exemplary flow-chart of oocyte denudation.

FIG. 4 is an exemplary table of metabolite formulations.

FIG. 5 is an exemplary flow-chart for preparing a granulosa co-culture.

FIG. 6A is an exemplary embodiment of a co-cultured second biological sample.

FIG. 6B is an exemplary embodiment of a control group culture of a second biological sample.

FIG. 6C is an exemplary embodiment of a co-cultured oocyte.

FIG. 6D is an exemplary embodiment of a control culture of immature oocytes.

FIG. 7A is a flow diagram of an exemplary method for inducing human oocyte maturation in vitro.

FIG. 7B is an exemplary flow diagram illustrating a method for oocyte rescue in vitro post stimulation.

FIG. 8 is a block diagram of a computing system that can be used to implement any one or more of the methodologies disclosed herein and any one or more portions thereof.

FIG. 9 shows an experimental workflow of ovaroid formation. First, barcoded transcription factor (TF) expression vectors were integrated into FOXL2-T2A-tdTomato reporter human induced pluripotent stem cells (hiPSCs). After induction of TF expression, cells positive for tdTomato and granulosa-related surface markers were sorted, and the barcodes were sequenced. The top TFs based on barcode enrichment were selected for further characterization by combinatorial screening and bulk RNA-seq. Next, monoclonal hiPSC lines were generated that inducibly express the top TFs and generate granulosa-like cells with high efficiency. Granulosa-like cells from these lines were further evaluated for estradiol production in response to follicle-stimulating hormone (FSH). Finally, they were aggregated with human primordial germ cell-like cells (hPGCLCs) to form ovaroids. These ovaroids produced estradiol and progesterone, formed follicle-like structures, and supported hPGCLC maturation as measured by immunofluorescence microscopy and scRNA-seq.

FIG. 10A is a schematic of the experimental co-culture IVM approach. hiPSCs are differentiated using inducible transcription factor overexpression to form ovarian supporting cells (OSCs). Immature human cumulus oocyte complexes (COCs) are obtained from donors in the clinic after undergoing abbreviated gonadotropin stimulation. In the lab, embryology dishes are prepared including OSCs seeding as required, and COCs are introduced for IVM co-culture. Oocyte maturation and morphological quality are assessed after 24-28 hours IVM co-culture, and samples are banked for analysis or utilized for embryo formation.

FIG. 10B is a representative image of a co-culture setup at time of plating containing human COCs (n=5) and 100,000 OSCs. Scale bar is 100 μm. COCs with expanded and unexpanded cumulus are seen with surrounding OSCs in suspension culture.

FIG. 11A shows the maturation rate of oocytes after 24-28 hour IVM experiments in Experiment 1, including oocyte co-culture with OSCs, or in Media Control. n indicates the number of individual oocytes in each culture condition. Error bars indicate mean±SEM. p-value is derived from unpaired t-test comparing OSC-IVM to Media Control condition.

FIG. 11B shows the Total Oocyte Score (TOS) generated from imaging analysis of MII oocytes after 24-28 hour IVM experiments. n indicates the number of individual MII oocytes analyzed. Median (dashed line) and quartiles (dotted line) are indicated. An unpaired t-test indicated no significant (p=0.2909) difference between the means. Due to low numbers of retrieved oocytes per donor, oocytes could not be consistently split between both conditions analyzed. Groups contain oocytes from predominantly non-overlapping donor cohorts and pairwise comparisons are not utilized.

FIG. 12A shows the maturation rate of oocytes after 28-hour IVM experiments in Experiment 2, including oocyte co-culture with OSCs or in Commercially available IVM Control. n indicates the number of individual oocytes in each culture condition. Error bars indicate mean±SEM. p-value derived from paired t-test comparing Experimental OSC-IVM to Control Condition (Commercial IVM Control).

FIG. 12B shows the Total Oocyte Score (TOS) generated from imaging analysis of MII oocytes after 28-hour IVM experiments. n indicates the number of individual MII oocytes analyzed. Median (dashed line) and quartiles (dotted line) are indicated. An unpaired t-test indicated no significant (p=0.9420) difference between the means. COCs from each donor were randomly and equitably distributed between control and intervention to allow for pairwise statistical comparison.

FIG. 13A shows the embryo formation outcomes after 28-hour IVM experiments in the subset of oocytes utilized for embryo formation in Experiment 2, including oocyte co-culture with OSCs or in Commercially available IVM Control. Error bars indicate mean±SEM. Results are displayed as a percentage of total COCs treated in the group. Outcomes for fertilization, cleavage, blastocyst formation, high quality blastocyst formation and euploid blastocyst formation are assessed for both IVM conditions.

FIG. 13B shows representative images of embryo formation in OSC-IVM versus Commercial IVM conditions at day 3 cleavage, as well as day 5, 6, and 7 of blastocyst formation. Embryos that were of suitable vitrification quality are labeled as “usable quality blast” and were utilized for trophectoderm biopsy.

FIG. 14A is a schematic of the experimental co-culture IVM approach. hiPSCs were differentiated using inducible transcription factor overexpression to form ovarian support cells (OSCs). Human oocytes were obtained from donors in the clinic after undergoing standard gonadotropin stimulation, and immature oocytes (GV and MI) identified after denuding were allocated to this research study. In the embryology lab, dishes were prepared including OSCs seeding as required, and immature oocytes were introduced for IVM co-culture. Oocyte maturation and health were assessed after 24-28 hours IVM co-culture, and oocyte samples were banked for further analyses.

FIG. 14B is a representative image of co-culture setup at time of plating containing immature human oocytes (n=3) and OSCs. Scale bar: 200 μm. Denuded GV oocytes are seen with surrounding OSCs in suspension culture.

FIG. 15A shows the maturation rate of oocytes after 24-28 hour IVM experiments, including oocyte co-culture with OSCs (OSC-IVM), or in Media Control (Media-IVM). n indicates the number of individual oocytes in each culture condition. Error bars indicate mean±SEM. p-value derived from unpaired t-test comparing Experimental OSC-IVM to Control Media-IVM. Due to low numbers of retrieved oocytes per donor, each group contains oocytes from predominantly non-overlapping donor groups and pairwise comparisons are not utilized.

FIG. 15B shows Total Oocyte Scores (TOS) generated from imaging analysis of MII oocytes after 24-28 hour IVM experiments. n indicates the number of individual MII oocytes analyzed. Median (dashed lines) and quartiles (dotted lines) are indicated. Unpaired t-test indicated no significant (ns, p=0.5725) difference between the means.

FIG. 16A shows representative images of MII oocytes after 28-hour IVM co-culture with OSCs, stained with fluorescent alpha-tubulin dye (cyan) to visualize the meiotic spindle. Blue lines transecting the middle of the PB1 and the spindle assembly from the oocyte center were used to derive the PB1-spindle angle. PB1-spindle angle ranges are indicated above. An example of an MII with a missing spindle is provided from the Media-IVM condition.

FIG. 16B shows quantification of the angle between the PB1 and spindle, derived from oocyte fluorescence imaging analysis (as in A). n= indicates the number of individual oocytes analyzed from each condition. Number of MII oocytes with no spindle assembly observed is also indicated below the axis labels. Median (dashed line) and quartiles (dotted line) are indicated. ANOVA statistical analysis found no significant difference (ns, p=0.1155) between the means of each condition.

FIG. 17A shows UMAP projections of oocyte transcriptomes with symbols colored by experimental batch, experimental condition (OSC-IVM, Media-IVM, IVF-MII), oocyte maturation state, and Leiden cluster. Each symbol represents one oocyte. n=81 oocytes.

FIG. 17B shows UMAP projections colored by scores for each of the gene marker sets (GV and IVF MII).

FIG. 17C shows UMAP projection generated from the scores of cells for each of the two signature marker sets (GV vs IVF MII), colored by experimental condition, oocyte maturation state, and Leiden cluster.

FIG. 17D shows quantification of oocytes in each maturation outcome (GV, MI and MII) by experimental condition (IVM or IVF), with color distribution indicating percentage of population in each Leiden cluster. Striped bars are utilized to denote clusters with predominantly IVF-like characteristics.

FIG. 18A shows immunofluorescence images of human ovaroid (F66/N.R1.G.F #4 granulosa-like cells+hPGCLCs) sections at days 2, 4, 14, and 32 of culture, stained for FOXL2 (granulosa), OCT4 (germ cell/pluripotent), and DAZL (mature germ cell). Scale bars are 40 μm.

FIG. 18B shows mouse ovaroid (fetal mouse ovarian somatic cells+hPGCLCs) sections stained as in FIG. 18A. Scale bars are 40 μm.

FIG. 18C shows the fraction of OCT4+ and DAZL+ cells relative to the total (DAPI+) over time in human ovaroids and mouse xenovaroids. Counts were performed at 11 time points on images from 2 replicates of human ovaroids (F66/N.R1.G.F #4 and F66/N.R2 #1 granulosa-like cells+hPGCLCs) and 1 replicate of mouse xeno-ovaroids.

FIG. 18D shows immunofluorescence images of human ovaroid (F66/N.R2 #1 granulosa-like cells+hPGCLCs) sections at days 4 and 8 of culture, stained for SOX17 (germ cell), TFAP2C (early germ cell), and AMHR2 (granulosa). Scale bars are 40 μm.

FIG. 18E shows DAZL and OCT4 expression observed by immunofluorescence in day 16 ovaroids. Some DAZL+OCT4− cells (magenta arrows) are visible, as well as DAZL+OCT4+ cells (cyan arrows). Ovaroids are also beginning to form follicle-like morphology (yellow arrows). Scale bars are 40 μm.

FIG. 19A shows day 35 human ovaroid (F66/N.R1.G #7+hPGCLC) sections stained for FOXL2, OCT4, and AMHR2. Scale bars are 40 μm. Follicle-like structures are marked with yellow triangles.

FIG. 19B shows a whole-ovaroid view of follicle-like structures in human ovaroids (F66/N.R1.G #7). Scale bars are 1 mm.

FIG. 19C shows a section of human ovaroid (F66/N.R1.G.F #4+hPGCLC) at day 70 of culture, stained for FOXL2, NR2F2, and AMHR2, showing multiple small follicles (yellow triangles) consisting single layers of FOXL2+AMHR2+ cells. NR2F2+ cells are interspersed between these. Scale bars are 100 μm.

FIG. 19D shows a section of human ovaroid (F66/N.R2 #1+hPGCLC) at day 70 of culture, stained for FOXL2, NR2F2, and AMHR2, showing an antral follicle consisting of FOXL2+AMHR2+ granulosa-like cells arranged in several layers around a central cavity. NR2F2 staining is visible outside of the follicle (marked ‘Stroma’). Scale bars are 100 μm.

FIG. 20A shows the expression (log 2 CPM) of selected granulosa (FOXL2), stroma/theca (NR2F2), and germ cell (PRDM1) markers. Expression is from scRNA-seq analysis of ovaroids (F66/N.R1.G.F #4 granulosa-like cells+hPGCLCs). Data from all samples (days 2, 4, 8, and 14) were combined for joint dimensionality reduction and clustering.

FIG. 20B shows Leiden clustering of four main clusters; the expression (log 2 CPM) of marker genes is plotted for each cluster from the scRNA-seq analysis of ovaroids as in FIG. 20A.

FIG. 20C shows the mapping of cells onto a human fetal ovary reference atlas (Garcia-Alonso et al., 2022) and assignment of cell types based on the scRNA-seq analysis described in FIG. 20A.

FIG. 20D shows the proportion of somatic cell types, germ cells, DAZL+ cells, and DDX4+ cells in ovaroids from each day based on the scRNA-seq analysis described in FIG. 20A.

FIG. 21A shows denuded oocytes from standard of care.

FIG. 21B shows COCs from minimal stimulation.

FIG. 21C shows OSC-IVM statistically significantly improves oocyte maturation rates.

FIG. 22A shows morphological quality of oocytes grown in culture with OSCs-IVM.

FIG. 22B shows the angle between the PB1 and the spindle of oocytes grown in culture with OSCs-IVM.

FIG. 22C shows the high similarity of oocytes grown in culture with OSCs-IVM to in vivo MII oocytes.

FIG. 22D shows the high similarity of oocytes grown in culture with OSCs-IVM to in vivo MII oocytes.

FIG. 23A shows the oocyte degradation rate from a toxicity assessment of OSCs-IVM product.

FIG. 23B shows the fertilization and blastocysts generation of OSCs-IVM product.

FIG. 24 shows that OSC-IVM oocytes show similar stress and cell cycle-related differential gene expression relative to IVF-MII control compared to Media-IVM oocytes. Gene expression values of oocytes for different developmental states (GV, MI or MII) for each experimental condition (OSC-IVM, Media-IVM, IVF-MII) are grouped for analysis, with each row representing a specific group. Relative (top panel) and absolute (bottom panel) gene expression are shown for each group for specific genes with known roles in cell cycle, stress, and meiosis, with each column indicating a specific gene. Samples are ordered on the y-axis utilizing unsupervised hierarchical clustering (UHC) for the selected genes, as a measure of relative similarity. The drawings are not necessarily to scale and may be illustrated by phantom lines, diagrammatic representations, and fragmentary views. In certain instances, details that are not necessary for an understanding of the embodiments or that render other details difficult to perceive may have been omitted.

### DEFINITIONS

Unless otherwise defined herein, scientific, and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

As used herein, the term “about” refers to a value that is within 10% (10%, 9%, 8%, 7%, 6%, 5%, 4%,3%, 2%, 1%, or less) above or below the value being described. For instance, the phrase “about 50 mg” refers to a value between and including 45 mg and 55 mg.

As used herein, the term “assisted reproductive technology” or “ART” refers to a fertility treatment in which one or more female gametocytes (oocytes) or gametes (ova) are manipulated ex vivo so as to promote the formation of an embryo that can, in turn, be implanted into a subject in an effort to achieve pregnancy. For example, in some embodiments, an oocyte retrieved from a subject undergoing an ART procedure may be matured in vitro using, e.g., co-culturing methodologies described herein. In some embodiments, upon the formation of a mature oocyte (ovum), the ovum may be treated with a sperm cells so as to promote the formation of a zygote and, ultimately, an embryo. The embryo may then be transferred to the uterus of a female subject, for instance, using the compositions and methods in the art. Exemplary ART procedures include in vitro fertilization (IVF) and intracytoplasmic sperm injection (ICSI) techniques described herein and known in the art.

As used herein, the terms “subject” refers to an organism that receives treatment for a particular disease or condition as described herein. Examples of subjects and subjects include mammals, such as humans (e.g., a female human), receiving treatment for diseases or conditions that correspond to a reduced ovarian reserve or release of immature oocytes.

As used herein, the term “controlled ovarian hyperstimulation” refers to a procedure in which ovulation is induced in a subject, such as a human subject, prior to oocyte or ovum retrieval for use in embryo formation, for instance, by in vitro fertilization (IVF) or intracytoplasmic sperm injection (ICSI). Controlled ovarian hyperstimulation procedures may involve administration of follicle-stimulating hormone (FSH), human chorionic gonadotropin (hCG), and/or a gonadotropin-releasing hormone (GnRH) antagonist to the subject so as to promote follicular maturation. Controlled ovarian hyperstimulation methods are known in the art and are described herein as they pertain to methods for inducing follicular maturation and ovulation in conjunction with assisted reproductive technology.

As used herein, the term “derived from” in the context of a cell derived from a subject refers to a cell, such as a mammalian ovum, that is either isolated from the subject or obtained from expansion, division, maturation, or manipulation (e.g., ex vivo expansion, division, maturation, or manipulation) of one or more cells isolated from the subject. For instance, an ovum is “derived from” a subject or an oocyte as described herein if the ovum is directly isolated from the subject or obtained from the maturation of an oocyte isolated from the subject, such as an oocyte isolated from the subject from about 1 day to about 5 days following the subject receiving ovarian hyperstimulation procedures (e.g., an oocyte isolated from the subject from about 2 days to about 4 days following ovarian hyperstimulation procedures).

As used herein, the term “dose” refers to the quantity of a therapeutic agent, such as a follicle stimulating agent described herein, that is administered to a subject for the treatment of a disorder or condition, such as to enhance oocyte maturation and/or release and promote retrieval and ex vivo maturation of viable oocytes. A therapeutic agent as described herein may be administered in a single dose or in multiple doses. In each case, the therapeutic agent may be administered using one or more unit dosage forms of the therapeutic agent. For instance, a single dose of 100 mg of a therapeutic agent may be administered using, e.g., two 50 mg unit dosage forms of the therapeutic agent. Similarly, a single dose of 300 mg of a therapeutic agent may be administered using, e.g., six 50 mg unit dosage forms of the therapeutic agent or two 50 mg unit dosage forms of the therapeutic agent and one 200 mg unit dosage form of the therapeutic agent, among other combinations. Similarly, a single dose of 900 mg of a therapeutic agent may be administered using, e.g., six 50 mg unit dosage forms of the therapeutic agent and three 200 mg unit dosage forms of the therapeutic agent or ten 50 mg unit dosage form of the therapeutic agent and two 200 mg unit dosage forms of the therapeutic agent, among other combinations.

As used herein, the term “follicular triggering period” refers to the timepoint for administering a follicular triggering agent. The timepoint for administering a follicular triggering agent (i.e., the follicular triggering period) to a female subject is on day 1, day 2, or day 3 of her menstrual cycle, with preference for day 2 of her menstrual cycle. However, if the female subject is taking a hormonal contraceptive, then the timepoint for administering a follicular triggering agent is 4-6 days (e.g., 4 days, 5 days, or 6 days) after consuming the last oral contraception pill, with preference for 5 days following the dosing of her last oral contraception pill.

As used herein, the term “follicle-stimulating hormone” (FSH) refers to a biologically active heterodimeric human fertility hormone capable of inducing ovulation in a subject. FSH may be purified from post-menopausal human urine or produced as a recombinant protein product. Exemplary recombinant FSH products include follitropin alfa (GONAL-F, Merck Serono/EMD Serono) and follitropin beta (PUREGON/FOLLISTIM, MSD/Scherig-Plough).

As used herein, the term “human chorionic gonadotropin” (hCG) refers to the polypeptide hormone that interacts with the luteinizing hormone chorionic gonadotropin receptor (LHCGR) to induce follicle maturation and ovulation. hCG may be purified from the urine of pregnant women or produced as a recombinant protein product. Exemplary recombinant hCG products include choriogonadotropin alfa (OVIDREL®, Merck Serono/EMD Serono).

As used herein, the term “in vitro fertilization” (IVF) refers to a process in which an ovum, such as a human ovum, is contacted ex vivo with one or more sperm cells so as to promote fertilization of the ovum and zygote formation. The ovum can be derived from a subject, such as a human subject, undergoing various ARTs known in the art. For instance, one or more oocytes may be obtained from the subject following injection of follicular maturation stimulating agents for controlled ovarian hyperstimulation procedures, e.g., from about 1 day to about 5 days prior after injection of said agents (such as from about one day to about 4 days after injection of follicular maturation stimulating agents to the subject). The ovum may also be retrieved directly from the subject, for instance, by transvaginal ovum retrieval procedures known in the art.

As used herein, the term “intracytoplasmic sperm injection” (ICSI) refers to a process in which a sperm cell is injected directly into an ovum, such as a human ovum, so as to promote fertilization of the ovum and zygote formation. The sperm cell may be injected into the ovum, for instance, by piercing the oolemma with a microinjector so as to deliver the sperm cell directly to the cytoplasm of the ovum. ICSI procedures useful in conjunction with the compositions and methods described herein are known in the art and are described, for instance, in WO 2013/158658, WO 2008/051620, and WO 2000/009674, among others, the disclosures of which are incorporated herein by reference as they pertain to compositions and methods for performing intracytoplasmic sperm injection.

As used herein, the terms “ovum” and “oocyte” refer to a haploid female reproductive cell or gamete. In the context of assisted reproductive technology as described herein, ova may be produced ex vivo by maturation of one or more oocytes isolated from a subject undergoing ART. Ova may also be isolated directly from the subject, for example, by transvaginal ovum retrieval methods described herein or known in the art. Ovum or oocyte as used in this disclosure may refer to a plurality of oocytes. An oocyte may be in complex with surrounding cells such as a cumulus-oocyte complex (COC).

As used herein, the terms “mature ova” and “mature oocyte” refer to one or more ovum or oocyte in metaphase II (MII)-stage of meiosis and typically has morphological or structural features consistent with metaphase II, such as a polar body and other features described herein.

As used herein, the terms “immature ovum” and “immature oocyte” refer to one or more ovum or oocyte that has not reached MII stage of meiosis. In some embodiments, an immature oocyte may be an oocyte including germinal vesicle (GV)-stage and/or metaphase I (MI)-stage oocytes as determined by morphological features and/or other indications known in the art.

As used herein, the term “oocyte maturation” refers to the process by which an immature oocyte developmentally transitions to a mature oocyte. Oocyte maturation occurs as immature oocytes undergo cell signaling events incurred by external and internal stimuli. External stimuli may be produced by neighboring cells or supporting cells described herein. Oocyte maturation may occur prior to the release of an oocyte and retrieval from a subject. Oocyte maturation may occur in vitro as a result of culturing methods and culture compositions described herein.

As used herein, an “ovarian support cell” (OSC) or “support cell” refers to one or more cells that promotes maturation of one or more oocytes. An OSC may be an ovarian granulosa cell (e.g., a type of granulosa cell described herein). Additionally or alternatively, an OSC may be an ovarian stroma cell (e.g., a type of stroma cell described herein). An OSC may form a cumulus-oocyte complex (COC) with an oocyte. An OSC may be generated from an exogenous source, such as from induced pluripotent stem cells (iPSCs), e.g., human induced pluripotent stem cells (hiPSCs), as described herein. An OSC may be applied to a retrieved oocyte using in vitro cell culture methods and compositions described herein. An OSC may be a mixture of two or more cell types. An OSC may be a mixture of stroma cells and granulosa cells such that the mixture is approximately a 1:1 population of stroma cells and granulosa cells. An OSC may be a mixture of stroma cells and granulosa cells such that one cell type is in higher relative abundance compared to one or more cell types such that the mixture is approximately a 2:1 population, a 3:1 population, a 4:1 population, a 5:1 population, among other possible population distributions. An OSC may be a mixture of stroma cells and granulosa cells such that one cell type is more abundant in the mixture (e.g., 90% stroma cells and 10% granulosa cells, 80% stroma cells and 20% granulosa cells, 70% stroma cells and 30% granulosa cells, 60% stroma cells and 40% granulosa cells, 40% stroma cells and 60% granulosa cells, 30% stroma cells and 70% granulosa cells, 20% stroma cells and 80% granulosa cells, or 10% stroma cells and 90% granulosa cells, among other possible distributions). In some embodiments, an OSC may be a mixture of stroma cells and granulosa cells in combination with one or more additional cell types.

As used herein, an “ovarian stroma cell” or a “stroma cell” is a cumulus cell surrounding the oocyte to ensure healthy oocyte and subsequent embryo development. An ovarian stroma cell may form a COC with an oocyte. An ovarian stroma cell may express markers consistent with a stroma subtype such as nuclear receptor subfamily 2 group F member 2 (NR2F2), which can be detected by methods known in the art. An ovarian stroma cell may be a steroidogenic stroma cell. An ovarian stroma cell may be produced from differentiated hiPSCs as described herein.

As used herein, a “steroidogenic stroma cell” is a stroma cell that may produce one or more steroids such as estradiol, progesterone, or a combination thereof. One or more steroids may be produced in response to hormonal stimulation, such as by FSH, androstenedione, or a combination thereof. One or more steroids may be secreted.

As used herein, an “ovarian granulosa cell” or a “granulosa cell” is a cumulus cell surrounding the oocyte to ensure healthy oocyte and subsequent embryo development. An ovarian granulosa cell may form a COC with an oocyte. An ovarian granulosa cell may express markers consistent with a granulosa subtype such as FOXL2, CD82 and/or follicle-stimulating hormone receptor (FSHR), which can be detected by methods known in the art. An ovarian granulosa cell may be a steroidogenic granulosa cell.

An ovarian granulosa cell may be produced from differentiated hiPSCs as described herein.

As used herein, a “steroidogenic granulosa cell” is a granulosa cell that may produce one or more steroids such as estradiol, progesterone, or a combination thereof. One or more steroids may be produced in response to hormonal stimulation, such as by FSH, androstenedione, or a combination thereof. One or more steroids may be secreted.

As used herein, the term “biological sample” or “sample” refers to a specimen (e.g., blood, blood component (e.g., serum or plasma), urine, saliva, amniotic fluid, cerebrospinal fluid, tissue (e.g., placental or dermal), pancreatic fluid, chorionic villus sample, hair, oocyte, ovum, and/or cells isolated from a subject.

As used herein, the terms “oral contraceptive treatment,” “oral contraception,” “contraception,” or “birth control pill” refers to a hormonal method of treatment typically used to prevent pregnancy. Oral contraceptive treatment may block the release of oocytes from the ovaries and may contain hormones including estrogen and progestin.

As used herein, the term “ovarian reserve” refers to the number of oocytes in a subject's ovaries and the quality of said oocytes. The ovarian reserve naturally declines with age and/or medical conditions described herein. Subjects with a diminished ovarian reserve may seek IVF or other ARTs to achieve a successful pregnancy. Levels of anti-Müllerian hormone (AMH), as described herein, may be indicative of a subject's ovarian reserve.

As used herein, the term “stimulation protocol” refers to the process of administering to the subject one or more follicular triggering agents during a follicular triggering period.

As used herein, the terms “follicular triggering agent” or “triggering agent” refer to a chemical or biological composition that stimulates release of oocytes from the ovaries during ovulation. Follicular triggering agents may include hormones such as human chorionic gonadotropin and follicle-stimulating hormone. As used herein, the term “induced pluripotent stem cells” (iPSCs) refer to artificial stem cells that derive from reprogrammed and otherwise manipulated harvested somatic cells. iPSCs may differentiate into other cell types including ovarian support cells or granulosa cells via methods known in the art and methods described herein. iPSCs may be humans (hiPSCs) or iPSCs from, e.g., other mammalian sources.

As used herein, the term “cell culture” refers to laboratory methods that enable in vitro cell proliferation and/or cultivation of prokaryotic or eukaryotic cell types.

As used herein, the term “co-culture” refers to a type of cell culture method in which more than one cell type or cell populations are cultivated with some degree of contact between them. In a typical co-culture system, two or more cell types may share artificial growth medium.

As used herein, the terms “adherent co-culture systems” or “adherent cell culture” refer to a cell culture arrangement by which cells are attached to a surface for proper growth and proliferation.

As used herein, the terms “suspension co-culture systems” or “suspension cell culture” refer to a cell culture arrangement by which cells are cultivated via dispersion in a liquid medium for proper growth and proliferation.

### DETAILED DESCRIPTION

Described herein are apparatuses, compositions, and methods for use in assisted reproductive technology (ART). For example, the apparatuses, compositions, and methods described herein are directed to follicle stimulation for ovarian release of oocytes and in vitro maturation of oocytes after follicle stimulation (i.e., post stimulation).

Advantageously, the methods described herein enable the harvest and use of previously discarded oocytes for purposes of traditional in vitro fertilization (IVF) by performing in vitro maturation of immature oocytes via co-culture with ovarian support cells (e.g., ovarian granulosa and/or stroma cells).

The described in vitro maturation methods improve the ability to use these typically discarded immature oocytes in IVF procedures and may lead to a more cost-effective treatment strategy and reduced risk to a treated subject. For example, the methods can reduce the risk of systemic ovarian overstimulation for subjects seeking IVF procedures by requiring fewer hormone injections and/or lower doses of injected hormones than present IVF treatment options. Aspects of the present disclosure can be used to increase the overall pool of available healthy oocytes in women for use in IVF. Aspects of the present disclosure can also be used to significantly reduce hormone dosing in subjects during egg retrieval and improve oocyte quality in culture. This may greatly expand access to reproductive technology, make the duration of a single cycle significantly shorter and require fewer cycles overall to achieve pregnancy.

### I. Methods of Stimulating Oocyte Release

### A. Subject Selection

The methods of stimulating oocyte release described herein are directed to a subject seeking IVF treatment options. In general, a subject is a female with a low oocyte retrieval number or a subject with many immature oocytes. A subject may be between 20 and 45 years old, and a subject is typically 35 years of age or older. A subject may have a reduced ovarian reserve due to advancing age and/or a genetic or medical condition (e.g., polycystic ovarian syndrome (PCOS)) that leads to a reduced ovarian reserve. A subject may have an ovarian reserve of 20 or fewer oocytes such that a subject has 1 to 5 oocytes, 4 to 10 oocytes, 8 to 16 oocytes, or 15 to 20 oocytes, e.g., the subject has 1 oocyte, 2 oocytes, 3 oocytes, 4 oocytes, 5 oocytes, 6 oocytes, 7 oocytes, 8 oocytes, 9 oocytes, 10 oocytes, 11 oocytes, 12 oocytes, 13 oocytes, 14 oocytes, 15 oocytes, 16 oocytes, 17 oocytes, 18 oocytes, 19 oocytes, or 20 oocytes. A subject may have anti-Mullerian hormone (AMH) levels that are consistent with reduced ovarian reserve. A subject may have their AMH levels measured by a blood test and other methods known in the art. A subject may have AMH levels between 1 and 6 ng/mL (e.g., 1-2 ng/mL, 2-4 ng/mL, or 4-6 ng/mL; e.g., 1 ng/mL, 2 ng/mL, 3 ng/mL, 4 ng/mL, 5 ng/mL, or 6 ng/mL). A subject may have measured estradiol levels between 20 and 50 μg/mL (e.g., 20-30 μg/mL, 25-35 μg/mL, 30-40 μg/mL, 35-45 μg/mL, or 40-50 μg/mL; e.g., 20 μg/mL, 21 μg/mL, 22 μg/mL, 23 μg/mL, 24 μg/mL, 25 μg/mL, 30 μg/mL, 35 μg/mL, 40 μg/mL, 45 μg/mL, or 50 μg/mL).

A physician or skilled practitioner may evaluate a subject for the methods of stimulating oocyte release by taking a biological sample from the subject. A biological sample may include a laboratory specimen held by a biorepository for research. In some embodiments, a biological sample may include bodily fluids including blood, saliva, urine, semen (seminal fluid), vaginal secretions, cerebrospinal fluid (CSF), synovial fluid, pleural fluid (pleural lavage), pericardial fluid, peritoneal fluid, amniotic fluid, saliva, nasal fluid, optic fluid, gastric fluid, breast milk, cell culture supernatants, and the like. A biological sample may include a medical diagnosis, user input describing how a user is feeling and/or a symptomatic complaint, information collected from a wearable device pertaining to a user and the like. For example, a biological sample may include information obtained from a visit with a medical professional such as a health history. In yet another non-limiting example, a biological sample may include information such as data collected from a wearable device worn by a user and designed to collect information relating to a user's sleep patterns, exercise patterns, and the like. In an embodiment, a biological sample collected at a particular date and/or time of a user's menstrual cycle. For instance, and without limitation, a biological sample may be collected on the second day of a user's menstrual cycle to evaluate one or more hormone levels. The biological sample may be utilized to determine markers of a subject's ovarian reserve that may be measured by a subject's AMH levels and/or other hormone levels or other indications. AMH levels of 1 ng/mL or less may be used to indicate a low ovarian reserve. A subject with a low ovarian reserve may have measured AMH levels of 1.0 ng/mL, 0.9 ng/mL, 0.8 ng/mL, 0.7 ng/mL, 0.6 ng/mL, 0.5 ng/mL, 0.4 ng/mL, 0.3 ng/mL, 0.2 ng/mL, or 0.1 ng/mL. Other biological samples that may be utilized to determine one or more markers of a subject's overall health include without limitation menstrual cycle progression, and/or monitor circulating hormone levels such as estradiol (E2), luteinizing hormone (LH), follicle-stimulating hormone (FSH), progesterone (P4), estrone (E1), estriol (E3), testosterone, androgens, dehydroepiandrosterone (DHEA), triiodothyronine (T3), tetraiodothyronine (T4), calcitonin, melatonin, insulin, cortisol, human growth hormone (HGH), adrenaline levels, and other hormones.

Other biological sample data taken from a subject includes at least an oocyte. As used in this disclosure, “biological sample data” is data that provides a characterization of the biological, genetic, biochemical and/or physiological properties, compositions, or activities of biological samples. In some embodiments, an oocyte may be an immature oocyte. An “immature oocyte” as used in this disclosure is a one or more immature reproductive cells originating in the ovaries. In some embodiments, an immature oocyte may be an oocyte including GV and/or MI oocytes. In some embodiments, an immature oocyte may be a plurality of oocytes. An immature oocyte may be immature cumulus-oocyte complexes (COCs) taken from the subject. As used in this disclosure, a “cumulus-oocyte complex” is an oocyte surrounded by specialized granulosa cells. As used in this disclosure, a “specialized granulosa cell” is a cumulus cell surrounding the oocyte to ensure healthy oocyte and embryo development. In some embodiments, the immature oocyte may contain an oocyte wherein the specialized granulosa cell is added to mature the oocyte in a cell culture (e.g., a co-culture) and thus create a COC.

In some embodiments of the method, the biological sample may be extracted from the user through an extraction device. An “extraction device” is a device and/or tool capable of obtaining, recording and/or ascertaining a measurement associated with a sample. The extraction device may include a needle, syringe, vial, lancet, Evacuated Collection Tubes (ECT), tourniquet, vacuum extraction tube systems, any combination thereof and the like. For example, the extraction device may comprise a butterfly needle set. Data from a biological sample may include measurements, for example, of serum calcium, phosphate, electrolytes, blood urea nitrogen and creatinine, uric acid, and the like.

In an embodiment of the method, biological sample information of a subject may be obtained from an ultrasound. An “ultrasound,” as used in this disclosure, is any procedure that utilizes sound waves to generate one or more images of a user's body. For example, an ultrasound may be utilized to obtain an image of a subject's reproductive organs and/or tissues. In an embodiment, an ultrasound may be performed at a particular time of a subject's menstrual cycle. For example, a subject may receive an ultrasound on day 2 of her cycle and this may be utilized to determine follicle size and/or follicle count. Selection of a stimulation protocol and/or adjustment to a stimulation protocol may be made utilizing this information. For example, a subject with an ultrasound that shows PCOS may have a dose adjustment made to one or more medications received and/or utilized during a stimulation protocol. In addition, the length of her stimulation protocol may be modified based on her PCOS diagnosis. In an embodiment, an ultrasound may be repeated one or more times throughout a subject's stimulation protocol, and information obtained may be utilized to adjust her stimulation protocol in real time.

### B. Oocyte Stimulation Protocols

A physician or skilled practitioner may determine the stimulation protocol of oocyte release directed to a subject using the described biological parameters. Such biological parameters include hormone levels (e.g., baseline hormone levels and/or hormone levels due to use of contraceptives), subject anatomy (e.g., follicle size, follicle count, ovarian morphology, and/or uterine morphology), among other biological parameters known to a skilled practitioner. A skilled practitioner may administer a stimulation protocol with any one or a combination of triggering agents, or compositions directed to stimulate follicular maturation and oocyte release, described herein.

Hormone levels or concentrations of other relevant compounds of the biological sample may include estradiol (E2), luteinizing hormone (LH), follicle-stimulating hormone (FSH), progesterone (P4), estrone (E1), estriol (E3), testosterone, androgens, dehydroepiandrosterone (DHEA), triiodothyronine (T3), tetraiodothyronine (T4), calcitonin, melatonin, insulin, cortisol, human growth hormone (HGH), adrenaline levels and the like. In some embodiments, the measurement of hormone levels may be based on blood analysis of the biological sample. For example, blood analysis may include plasma hormone analysis techniques. In some embodiments, measurement of hormone levels may be based on saliva hormone testing techniques. Measurement of hormone levels may be based on other forms of analysis such as hair, urine, and any other form of biological samples described throughout this disclosure. A subject may have a baseline serum level of estradiol from about 30 μg/mL to about 60 μg/mL (e.g., from about 30 μg/mL to about 45 μg/mL, from about 40 μg/mL to about 55 μg/mL, or from about 45 μg/mL to about 60 μg/mL; e.g., about 30 μg/mL, about 35 μg/mL, about 40 μg/mL, about 45 μg/mL, about 50 μg/mL, about 55 μg/mL, or about 60 μg/mL) prior to the follicular triggering period. A subject may have a baseline serum level of progesterone from about 0.5 ng/mL to about 2.5 ng/mL (e.g., from about 0.5 ng/mL to about 1.0 ng/mL, from about 1.0 ng/mL to about 1.5 ng/mL, from about 1.5 ng/mL to about 2.0 ng/mL, or from about 2.0 ng/mL to about 2.5 ng/mL; e.g., about 1.0 ng/mL, about 1.5 ng/mL, about 2.0 ng/mL, or about 2.5 ng/mL) prior to the follicular triggering period.

Additionally, a subject's contraception (e.g., hormonal contraception) usage may affect assignment of a stimulation protocol. Consideration for contraception may aid in determining the follicular triggering period in the woman's menstrual cycle. For instance, and without limitation, a subject who is not using any form of contraception may begin her stimulation protocol with recombinant follicle stimulating hormone (rFSH) between the first and third day of her menstrual cycle, with preference for the second day of her menstrual cycle. In yet another non-limiting example, a subject who is using contraception may begin her stimulation protocol with rFSH 4-6 days (e.g., 4 days, 5 days, or 6 days) after consuming her last oral contraception pill, with preference for 5 days following the dosing of her last oral contraception pill. In an embodiment, rFSH stimulation may be utilized for 2 to 3 days (e.g., 2 days or 3 days), depending on a subject's tolerance, follicle size, and/or growth dynamics. After this 2- or 3-day window, a coasting period of 1 to 3 days (e.g., 1 day, 2 days, or 3 days) may be utilized to monitor follicle size and allow for further follicle maturation and development. A “coasting period,” as used in this disclosure, is any period of time when a medication used throughout a stimulation protocol is not administered and/or consumed. A coasting period may last for example for 1 day, 2 days, 3 days, or more if medically necessary. During a coasting period, a subject may continue to receive one or more ultrasounds to monitor her progression.

Once a follicle size has reached anywhere from between about 8-10 mm (e.g., 7.5 mm, 8 mm, 8.5 mm, 9 mm, 9.5 mm, 10 mm, 10.5 mm, or more), a subject may be triggered with a dose of a triggering agent, such as human chorionic gonadotropin (hCG). A “follicle measurement” as used in this disclosure, is any measurement of an ovarian follicle. A follicle may include any sac found in an ovary that contains an unfertilized egg. A follicle measurement may be obtained using any methodology as described herein, including for example an ultrasound, a manual measurement, an automated measurement and the like. In an embodiment, a double hCG injection may be utilized, to induce follicle maturation to prepare one or more follicles for retrieval. A double hCG injection may be two or three injections of hCG. A blood test for one or more hormone levels such as E2, P4, and LH may be performed on the trigger day of the double dose of hCG injection to monitor hormone levels. After the day of the double dose of hCG, one or more hormone levels may be measured such as for example with a blood test to determine and examine levels of E2, P4, and LH.

A “triggering agent” is a chemical that triggers cell generation in the ovaries. A triggering agent (e.g., a follicular triggering agent) may include any substance including any non-prescription and/or prescription product. A triggering agent (e.g., a follicular triggering agent) may include any one or combination of the non-limiting examples such as LUPRON DEPOT® (Abbott Laboratories, North Chicago, IL), Ganirelix (Ferring Pharmaceuticals, Saint-Prex, Switzerland), Cetrotide (Merck Global, Readington Township, NJ), GONAL-F® (Merck Global), FOLLISTIM® (Merck Global), BRAVELLE® (Ferring Pharmaceuticals), CLOMID® (Patheon Pharmaceuticals Inc., Waltham, MA), Serephene (Teva, Tel Aviv-Yafo, Israel), GLUCOPHAGE® (Merck Global), FORTAMET® (Mylan, Canonsburg, PA), PREGNYL® (Schering Plough, Kenilworth, NJ), NOVAREL® (Ferring Laboratories, Parsippany, NJ), Repronex (Ferring Pharmaceuticals), FACTREL® (Zoetis Canada Inc., Kirkland, Canada), MENOPUR® (Ferring Pharmaceuticals), and other drugs that induce cell generation in ovaries that one skilled in the art would understand as applicable. A triggering agent (e.g., a follicular triggering agent) may include human serum albumin, FSH, hCG, androstenedione, and doxycycline among other triggering agents known in the art.

In one embodiment, a subject may not receive a triggering agent (e.g., a follicular triggering agent) to stimulate oocyte production. In one embodiment, a subject may receive multiple injections of a triggering agent over 1 to 4 days (e.g., 1 day, 2 days, 3 days, or 4 days) but no more than 5 days in the preferred stimulation protocol. A subject may receive multiple injections over multiple days such that a subject receives five dose injections of one or multiple triggering agents. For example, a subject may receive three days of stimulation using 300 IU to 700 IU of rFSH per injection (e.g., 300-500 IU, 400-600 IU, 500-700 IU, 300-350 IU, 350-400 IU, 400-450 IU, 450-500 IU, 500-550 IU, 550-600 IU, 600-650 IU, 650-700 IU; e.g., 300 IU, 325 IU, 350 IU, 375 IU, 400 IU, 425 IU, 450 IU, 475 IU, 500 IU, 525 IU, 550 IU, 575 IU, 600 IU, 625 IU, 650 IU, 675 IU, or 700 IU) with one or more injections per day. A subject may receive injections of hCG as a triggering agent (e.g., a follicular triggering agent) using 200-700 μg or 2,500-10,000 IU hCG (e.g., 200-500 μg, 300-600 μg, 400-700 μg, 200-300 μg, 300-400 μg, 400-500 μg, 500-600 μg, or 600-700 μg), with a preferred stimulation dose of 500 μg. A subject may receive one or more injections of clomiphene citrate in combination with other triggering agents with a dose of 50-150 mg (e.g., 50-75 mg, 60-80 mg, 75-100 mg, 90-115 mg, 110-130 mg, 125-150 mg; e.g., 50 mg, 60 mg, 70 mg, 80 mg, 90 mg, 100 mg, 110 mg, 120 mg, 130 mg, 140 mg, 150 mg) of clomiphene citrate per injection.

Prior to receiving a triggering agent, a subject's serum may be evaluated for levels of hormones or other relevant compounds. A subject may have serum levels of estradiol from about 250 μg/mL to about 400 μg/mL (e.g., from about 250 μg/mL to about 275 μg/mL, from about 275 μg/mL to about 300 μg/mL, from about 300 μg/mL to about 325 μg/mL, from about 325 μg/mL to about 350 μg/mL, from about 350 μg/mL to about 375 μg/mL, or from about 375 μg/mL to about 400 μg/mL; e.g., about 250 μg/mL, about 260 μg/mL, about 270 μg/mL, about 280 μg/mL, about 290 μg/mL, about 300 μg/mL, about 310 μg/mL, about 320 μg/mL, about 330 μg/mL, about 340 μg/mL, about 350 μg/mL, about 360 μg/mL, about 370 μg/mL, about 380 μg/mL, about 390 μg/mL, or about 400 μg/mL) prior to receiving a triggering agent. A subject may have serum levels of progesterone from about 0.25 ng/mL to about 0.75 ng/mL (e.g., from about 0.25 ng/mL to about 0.35 ng/mL, from about 0.35 ng/mL to about 0.45 ng/mL, from about 0.45 ng/mL to about 0.55 ng/mL, from about 0.55 ng/mL to about 0.65 ng/mL, or from about 0.65 ng/mL to about 0.75 ng/mL; e.g., about 0.25 ng/mL, about 0.30 ng/mL, about 0.35 ng/mL, about 0.40 ng/mL, about 0.45 ng/mL, about 0.50 ng/mL, about 0.55 ng/mL, about 0.60 ng/mL, about 0.65 ng/mL, about 0.70 ng/mL, or about 0.75 ng/mL) prior to receiving a triggering agent. A subject may have serum levels of LH from about 1.0 mIU/mL to about 2.5 mIU/mL (e.g., from about 1.0 mIU/mL to about 1.5 mIU/mL, from about 1.5 mIU/mL to about 2.0 mIU/mL, or from about 2.0 mIU/mL to about 2.5 mIU/mL; e.g., about 1.0 mIU/mL, about 1.25 mIU/mL, about 1.5 mIU/mL, about 1.75 mIU/mL, about 2 mIU/mL, about 2.25 mIU/mL, or about 2.5 mIU/mL) prior to receiving a triggering agent. A subject may have serum levels of FSH from about 11 mIU/mL to about 14 mIU/mL (e.g., from about 11 mIU/mL to about 12 mIU/mL, from about 12 mIU/mL to about 13 mIU/mL, or from about 13 mIU/mL to about 14 mIU/mL; e.g., about 11 mIU/mL, about 12 mIU/mL, about 13 mIU/mL, or about 14 mIU/mL) prior to receiving a triggering agent.

The triggering agent (e.g., a follicular triggering agent) may be administered over a course of time to produce a follicle stimulation protocol that is a minimal stimulation protocol. The minimal stimulation protocol is configured by a skilled practitioner to trigger the release of a cell in the span of about 3 days. A “minimal stimulation protocol” is a stimulation process spanning over a shortened period of time, compared to average in vitro fertilization (IVF) stimulation protocols, to aid in inducing an ovary to produce an oocyte. Typically, the average span of time for a stimulation protocol using standard IVF is approximately 8-14 days. The minimal stimulation protocol may induce the release of a cell in a span of 8 days or less (e.g. 8 days or less, 7 days or less, 6 days or less, 5 days or less, 4 days or less, 3 days or less, 2 days or less, or 1 day; e.g., between 1-3 days, between 2-4 days, between 3-5 days, between 4-6 days, between 5-7 days, or between 6-8 days; e.g., 1 day, 2 days, 3 days, 4 days, 5 days, 6 days, 7 days, or 8 days), which is a shorted period of time compared to the 8-14 days of standard IVF stimulation protocols. The average time for performing a minimal stimulation protocol may be 2 days. The average time for performing a minimal stimulation protocol may be 3 days. The average time for performing a minimal stimulation protocol may be 4 days. The average time for performing a minimal stimulation protocol may be 5 days. The average time for performing a minimal stimulation protocol may be 6 days. In an embodiment, the minimal stimulation protocol may not require administration of a follicular triggering agent for successful retrieval and subsequent maturation of an oocyte. In an embodiment, the minimal stimulation protocol may include selecting a first triggering agent (e.g., a follicular triggering agent) and selecting a second triggering agent (e.g., a follicular triggering agent) as a function of a follicle measurement and/or other biological sample data.

### C. Oocyte Retrieval

Following follicular stimulation, oocytes (or a group of cells containing an oocyte) are retrieved from the subject. An “oocyte,” as used in this disclosure, is a reproductive cell originating from an ovary. Approximately 24-48 hours (e.g., between 24-32 hours, between 32-40 hours, between 40-48 hours; e.g., about 24 hours, about 28 hours, about 32 hours, about 36 hours, about 40 hours, about 44 hours, about 48 hours) after final dose of triggering agent (e.g., a follicular triggering agent) that is administered, a subject may undergo an oocyte retrieval. On the day of oocyte retrieval, a blood test for one or more hormone levels such as E2, LH, FSH and/or P4 may be performed to ensure quality metrics, hormone levels are within range, and/or that hCG dose was ingested. Hormone levels of E2 may be from about 300 μg/mL to about 450 μg/mL (e.g., from about 300 μg/mL to about 350 μg/mL, from about 350 μg/mL to about 400 μg/mL, or from about 400 μg/mL to about 450 μg/mL; e.g., about 300 μg/mL, about 325 μg/mL, about 350 μg/mL, about 375 μg/mL, about 400 μg/mL, about 425 μg/mL, or about 450 μg/mL) on the day of oocyte retrieval. Hormone levels of LH may be from about 3 mIU/mL to about 6 mIU/mL (e.g., from about 3 mIU/mL to about 4 mIU/mL, from about 4 mIU/mL to about 5 mIU/mL, or from about 5 mIU/mL to about 6 mIU/mL; e.g., about 3 mIU/mL, about 3.5 mIU/mL, about 4 mIU/mL, about 4.5 mIU/mL, about 5 mIU/mL, about 5.5 mIU/mL, or about 6 mIU/mL) on the day of oocyte retrieval. Hormone levels of FSH may be from about 6 mIU/mL to about 9 mIU/mL (e.g., from about 6 mIU/mL to about 7 mIU/mL, from about 7 mIU/mL to about 8 mIU/mL, or from about 8 mIU/mL to about 9 mIU/mL; e.g., about 6 mIU/mL, about 6.5 mIU/mL, about 7 mIU/mL, about 7.5 mIU/mL, about 8 mIU/mL, about 8.5 mIU/mL, or about 9 mIU/mL) on the day of oocyte retrieval. Hormone levels of P4 may be from about 0.5 ng/mL to about 1.5 ng/mL (e.g., from about 0.5 ng/mL to about 1.0 ng/mL, from about 0.75 ng/mL to about 1.0 ng/mL, from about 1.0 ng/mL to about 1.5 ng/mL, or from about 1.25 ng/mL to about 1.5 ng/mL; e.g., about 0.5 ng/mL, about 0.75 ng/mL, about 1.0 ng/mL, about 1.25 ng/mL, or about 1.5 ng/mL) on the day of oocyte retrieval.

Oocytes (or a group of cells containing an oocyte) are retrieved from the subject using methods known in the art. For example, oocytes may be retrieved via aspiration using a transvaginal ultrasound with a needle guide on the probe to suction released follicular contents. Follicular aspirates may then be examined using a dissection microscope and washed with HEPES media (G-MOPS Plus, Vitrolife®) and filtered with a 70-micron cell strainer (Falcon®, Corning). Oocytes and/or COCs are then transferred to culture dishes and media to begin co-culturing and appropriate controls, as described herein. Other retrieval methods may include an extraction device, such as a needle, syringe, vial, lancet, Evacuated Collection Tubes (ECT), tourniquet, vacuum extraction tube systems, any combination thereof and the like. For example, the extraction device may comprise a butterfly needle set.

A retrieved oocyte may include but is not limited to an immature oocyte, a mature oocyte, a group of one or more oocytes, a group of one or more cells, such as a cumulus oocyte complex, among other examples. A “cumulus oocyte complex” (COC) as used in this disclosure, is an oocyte containing one or more surrounding cumulus cells. A COC may contain an immature oocyte. A COC may contain a mature oocyte.

An “immature oocyte” as used in this disclosure is one or more immature reproductive cells originating in the ovaries. In some embodiments, an immature oocyte may be an oocyte including but not limited to germinal vesicle stage (GV) and metaphase I stage (MI) oocytes, as described further below. In some embodiments, an immature oocyte may be a plurality of oocytes. An immature oocyte may be immature cumulus-oocyte-complexes (COCs) taken from a subject. A “mature oocyte” as used in this disclosure, may be one or more mature oocytes in metaphase II stage (MII). Once retrieved, a COC may rest for 1 hour, 2 hours, 3 hours or more to allow for equilibration to in vitro conditions for in vitro maturation.

At the time of retrieval, any one or more of the retrieved oocytes or cells described herein may be appropriately frozen and stored using methods known in the art for future use, analysis, or experimentation. Additionally, any one or more of the retrieved oocytes or cells described herein may be used fresh (i.e., ready for immediate use such as use for in vitro maturation or any one or more analyses or experimentation described herein).

### II. Method of Oocyte Rescue

### A. Oocyte Denudation

Following oocyte retrieval methods described above, one or more COCs may require oocyte denudation. As described in this disclosure, “oocyte denudation” refers to the removal of cumulus cells or other cell types from the oocyte by means of mechanical separation, chemical separation, or combinations thereof. Several methods of oocyte denudation are known in the art. In some embodiments, denudation may occur in a IVM well, by gently mechanically disassociating cells by pipetting to remove most cumulus and/or granulosa cells. If enzymatic disassociation is needed, the cells may be transferred to a separate dish for hyaluronidase treatment. COCs may be stripped with stripper tips and washed in IVM media or MOPS plus media to clean the oocyte for imaging and if needed inactivate hyaluronidase. Stripper tips may include 200 micron and/or 400 microns for fine cleaning. In some embodiments, germinal vesical (GV)-stage) and metaphase I (MI)-stage oocytes may be formulated and utilized in cultivation following denudation of the COCs. Denuded COCs may be transferred to a separate culture dish for imaging.

### B. Co-Culturing Oocytes for In Vitro Maturation

i. Co-Culture Contents and Timing

In the methods described herein, an oocyte may be combined with a specialized granulosa cell and/or a specialized stroma cell in a co-culture. A “specialized granulosa cell” and a “specialized stroma cell” refers to a cumulus cell surrounding the oocyte to ensure healthy oocyte and embryo development. In some embodiments, the granulosa and/or stroma co-culture cells are sourced from human induced pluripotent stem cells (hiPSCs). As used in this disclosure, a “co-culture” is a cell cultivation set-up, in which two or more different populations of cells are grown with some degree of contact between them. In some embodiments, steroidogenic granulosa cells, derived from human induced pluripotent stem cells (hiPSCs), may be co-cultured with immature oocytes (COCs), thereby reconstituting the follicular niche in vitro to promote rapid and efficient oocyte maturation in a manner that reinforces oocyte health and developmental competence. As used in this disclosure, a “steroidogenic granulosa cell” is a granulosa cell expressing high levels of steroidogenic enzymes that produce estradiol. For example, a steroidogenic granulosa cell may be a mural granulosa cell extracted from the antral follicle. Applying steroidogenic granulosa cells in the co-cultures of COCs may increase oocyte maturation in vitro after egg/oocyte retrieval, allowing for utilization of all retrieved eggs/oocyte by directly supplying nutrients, raw materials, and mechanical support to oocytes throughout gametogenesis and folliculogenesis. Steroidogenic granulosa cells may grow and perform oocyte maturation of immature COCs in standard IVF and IVM media as described further below. This may increase the overall pool of available, healthy oocytes for use in IVF and reduce the number of ova/oocyte retrieval procedures a user is subjected to.

In some embodiments of the method, a cell culture may be formed by combining an immature oocyte with a specialized granulosa cell and/or a specialized stroma cell, which is added to mature the oocyte in the cell culture and thus create a COC after extraction of one or more oocytes following the minimal stimulation protocol. In an embodiment, one or more specialized granulosa cells and/or specialized stroma cells may be thawed during a resting period of one or more COCs. In an embodiment, anywhere from between 50,000-150,000 specialized granulosa cells (e.g., 50,000-60,000 cells, 60,000-70,000 cells, 70,000-80,000 cells, 80,000-90,000 cells, 90,000-100,000 cells, 100,000-110,000 cells, 110,000-120,000 cells, 120,000-130,000 cells, 130,000-140,000 cells, or 140,000-150,000 cells; e.g., 50,000 cells, 55,000 cells, 60,000 cells, 65,000 cells, 70,000 cells, 75,000 cells, 80,000 cells, 85,000 cells, 90,000 cells, 95,000 cells, 100,000 cells, 105,000 cells, 110,000 cells, 115,000 cells, 120,000 cells, 125,000 cells, 130,000 cells, 135,000 cells, 140,000 cells, 145,000 cells, or 150,000 cells) may be combined with a COC during culturing. In an embodiment, thawed specialized granulosa cells may be placed into a culture medium prior to COC retrieval, including anywhere from about 24-120 hours beforehand (e.g., about 24-48 hours, about 48-72 hours, about 72-96 hours, about 96-120 hours; e.g., about 24-36 hours, about 30-40 hours, about 36-48 hours, about 48-56 hours, about 56-72 hours, about 72-84 hours, about 80-96 hours, about 90-100 hours about 96-108 hours, about 108-120 hours; e.g., about 24 hours, about 30 hours, about 36 hours, about 42 hours, about 48 hours, about 56 hours, about 60 hours, about 65 hours, about 72 hours, about 78 hours, about 86 hours, about 92 hours, about 96 hours, about 102 hours, about 110 hours, about 115 hours, about 120 hours). A COC may be transferred into culture medium containing thawed specialized granulosa cells to form a group culture as described below in more detail. In an embodiment, a group culture may be cultured in an incubator ranging in time from anywhere between 12-48 hours (e.g., 12-16 hours, 12-20 hours, 18-24 hours, 18-36 hours, 24-36 hours, 36-48 hours; e.g., 12 hours, 16 hours, 20 hours, 24 hours, 28 hours, 32 hours, 36 hours, 40 hours, 44 hours, 48 hours). The co-culture may be conducted at a biologically suitable temperature, e.g., 37° C.

In some embodiments of the method, a retrieved oocyte, including immature cumulus-oocyte complexes, may be cultured in a group culture. A “group culture” is an extracted COC combined with one or more additional cells. An additional cell may include any cell grown together with an extracted COC. An additional cell may include a specialized stroma cell. An additional cell may include a specialized granulosa cell. In an embodiment, a group culture may be cultured and/or incubated for a particular length of time, such as from between 12-120 hours (e.g., 12-24 hours, 12-36 hours, 24-48 hours, 36-60 hours, 54-72 hours, 68-96 hours, 96-120 hours; e.g., 12 hours, 14 hours, 16 hours, 18 hours, 20 hours, 22 hours, 24 hours, 26 hours, 28 hours, 30 hours, 32 hours, 34 hours, 36 hours, 38 hours, 40 hours, 42 hours, 44 hours, 46 hours, 48 hours, 50 hours, 52 hours, 54 hours, 56 hours, 58 hours, 60 hours, 62 hours, 64 hours, 66 hours, 68 hours, 70 hours, 72 hours, 74 hours, 76 hours, 78 hours, 80 hours, 82 hours, 84 hours, 86 hours, 88 hours, 90 hours, 92 hours, 94 hours, 96 hours, 98 hours, 100 hours, 102 hours, 104 hours, 106 hours, 108 hours, 110 hours, 112 hours, 114 hours, 116 hours, 118 hours, or 120 hours). For example, group culturing may include culturing the COCs with a granulosa co-culture as described further below. In some embodiments, group culturing may include culturing a control group of COCs with no co-culture, as described further below. In some embodiments, a user may donate immature oocytes, such as GV-stage and MI-stage oocytes that may be used in medium as part of the group culture to help grow COCs. Oocyte donation may follow an oocyte retrieval process as discussed above.

A subject participating in oocyte donation may be different, or the same, from the subject related to the second biological sample containing immature COCs. In some embodiments, an oocyte donation subject may undergo a stimulation protocol as disclosed above.

In some embodiments, the maturity of the oocyte retrieved from the subject may dictate the length of time during which the oocyte is co-cultured with ovarian support cells (e.g., specialized granulosa cells and/or specialized stroma cells). For example, less mature oocytes (e.g., GV oocytes) may require longer co-culturing periods than oocytes at a more advanced stage of meiosis (e.g., MI oocytes).

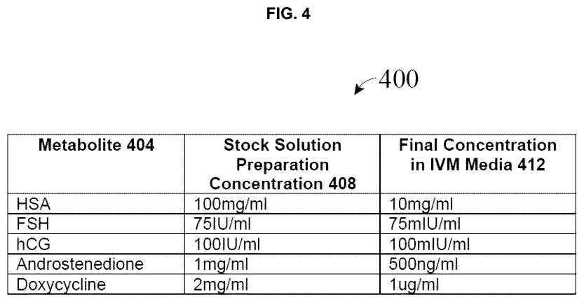

In some embodiments regarding the culture of oocytes, cell culture media may include LAG media (Medicult, CooperSurgical®). For example, LAG media may be used for the incubation of oocytes and/or COCs post-retrieval from minimal stimulation protocol. For example, a modified-Medicult IVM media may be used as a baseline control during the culturing process. In some embodiments, the cell culture media may include metabolites as exemplified in FIG. 4. For example, the modified-Medicult IVM media may include human serum albumin, FSH, hCG, androstenedione, doxycycline, or any combination thereof. Media may be equilibrated for about 18 to 24 hours (e.g., about 18 hours, about 20 hours, about 22 hours, about 24 hours) pre-culture in a standard sterile 37° C. incubator with O₂ (e.g., having a 1-10% O₂ atmosphere, such as 4-8% O₂ or 5-7% O₂, e.g., 6% O₂) and proper CO2 levels, which are known in the art. Co-cultures and specialized granulosa cell cultures may be adherent cell cultures in cell culture dishes or flasks. Co-cultures and specialized granulosa cell cultures may be suspension cell cultures in cell culture flasks. Cell culture materials and methods include standard sterile cell culturing methods known in the art. Cell morphology and cell viability may be evaluated via one or more established methods known in the art.

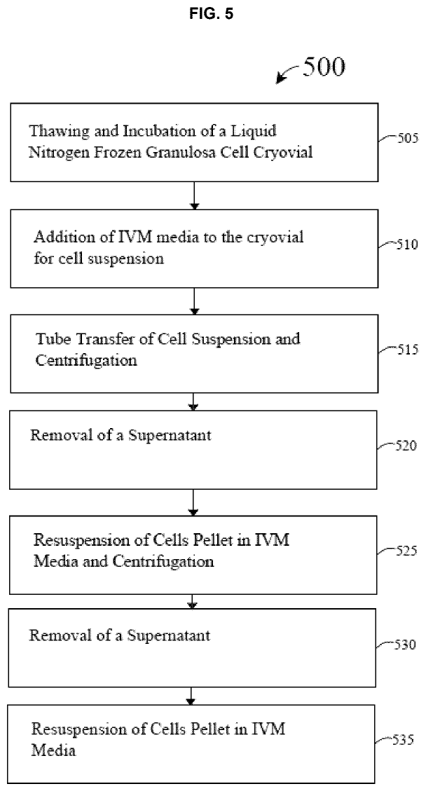

In some embodiments, co-culturing is performed in accordance with the steps outlined in FIG. 5.

For example, a population of ovarian support cells (e.g., ovarian granulosa cells) may be cryopreserved, thawed. In some embodiments, the ovarian support cells are centrifuged to form a cell pellet and are subsequently resuspended in media suitable for in vitro maturation. In some embodiments, the ovarian support cells are centrifuged one or more additional times and, each time, are resuspended in in vitro maturation media. The ovarian support cells may then be co-cultured with an oocyte obtained from the subject undergoing an ART procedure, thereby inducing oocyte maturation. ii. Granulosa cells from hiPSCs Specialized granulosa cells utilized in the methods described herein may be created from hiPSCs using transcription factor (TF)-directed protocols. In some embodiments, hiPSCs may be transformed with any one or more plasmids encoding one or more transcription factors. In some embodiments, hiPSCs may be transformed via electroporation, liposome-mediated transformation, viral-mediated gene transfer, among other cell transformation methodologies known in the art. In some embodiments, gene expression of desired transcription factors may be induced in a doxycycline-dependent manner. In some embodiments, transcription factors are constitutively expressed. In some embodiments, a plasmid or expression vector used for reprogramming hiPSCs may have a reporter gene such as a fluorescent protein. In some embodiments, hiPSCs may differentiate into stroma cells with induced expression of transcription factors including GATA4, FOXL2, or a combination thereof. In some embodiments, hiPSCs may differentiate into granulosa with induced expression of transcription factors including FOXL2, NR5A1, GATA4, RUNX1, RUNX2, or a combination thereof. In addition to a combination of one or more transcription factors of FOXL2, NR5A1, GATA4, RUNX1, and/or RUNX2, hiPSCs may differentiate into granulosa via expression of KLF2, TCF21, NR2F2, or a combination thereof.

Reprogramming of hiPSCs to stroma and/or granulosa may be determined by genotyping methods known in the art. Reprogramming of hiPSCs to granulosa may be determined by protein expression using any one or more methods known in the art. Differentiation of hiPSCs to stroma cells may be determined by relative expression of biomarkers typical of a stroma cell type including NR2F2 among others known in the art. Differentiation of hiPSCs to granulosa cells may be determined by relative expression of biomarkers typical of a granulosa cell type including AMHR2, CD82, FOXL2, FSHR, IGFBP7, KRT19, STAR, WNT4, or a combination thereof among other granulosa cell biomarkers known in the art. In some embodiments, reprogramming of hiPSCs to granulosa may be determined by production of growth factors and/or hormones including estradiol and progesterone that may adequately support in vitro maturation of retrieved oocyte via paracrine and juxtacrine cell signaling. In some embodiments, the resulting granulosa cells produce estradiol upon stimulation of androstenedione and FSH or forskolin. In some embodiments, the granulosa cells described herein may be produced in multiple batches. In some embodiments, the granulosa cells may be frozen and thawed prior to co-culture methods. In some embodiments, the granulosa cells were freshly differentiated prior to in vitro maturation method. In some embodiments, the granulosa cells may be seeded and equilibrated for 2-8 hours (e.g., 2-3 hours, 2-4 hours, 3-4 hours, 4-6 hours, 5-7 hours, 6-8 hours; e.g., 2 hours, 3 hours, 4 hours, 5 hours, 6 hours, 7 hours, 8 hours) before the addition of oocytes for in vitro maturation.

In some embodiments, a subject may donate hiPSCs. hiPSCs donation may follow an oocyte retrieval process as discussed above. A subject participating in hiPSCs donation may be different, or the same, from the subject from which the oocyte was retrieved. In some embodiments, a hiPSC donor may undergo a stimulation protocol as disclosed above.

In some embodiments, hiPSCs, granulosa cells, cumulus cells, oocytes, GV-stage oocytes, MI-stage oocytes, MII-stage oocytes and all other types of cells described through this disclosure may be lysed, extracted for genomic material and flash frozen for further manipulation and/or analysis (e.g., for analysis as part of an omics data collection technique described in Section II(C)(iii), below). For example, cells may undergo enzymatic cell lysis using enzymes such as lysozyme, lysostaphin, zymolase, cellulose, protease or glycanase, and the like. Other lysis methods may be applied such as chemical lysis, detergent lysis, alkaline lysis, mechanical lysis, thermal lysis, acoustic lysis, physical lysis, non-mechanical lysis and other lysis methods known in the art. In some embodiments, culture media may be flash frozen. Freezing methods may include using a cryoprotective agent such as dimethyl sulfoxide and/or any other freezing method known in the art.

iii. Transgenic Granulosa Cells

Specialized transgenic granulosa cells utilized in the methods described herein may be produced using Clustered Regularly Interspaced Short Palindromic Repeats (CRISPR) technology. “CRISPR” is programmable technology that targets specific stretches of genetic code to edit DNA at precise locations. CRISPR technology may include CRISPR-CAS 9. Cas9 (or “CRISPR-associated protein 9”) is an enzyme that uses CRISPR sequences as a guide to recognize and cleave specific strands of DNA that are complementary to the CRISPR sequence, allowing for the insertion of exogenous nucleic acids into a cell's genome. For example, CRISPR-based gene editing techniques can be used to introduce, into an iPSC genome, one or more genes encoding for factors that induce differentiation into ovarian support cells (e.g., ovarian granulosa cells). These factors include, e.g., FOXL2, NR5A1, GATA4, RUNX1, and RUNX2.

Exemplary CRISPR systems include those that utilize a Cas9 enzyme. Cas9 enzymes, together with CRISPR sequences, form the basis of a technology known as CRISPR-Cas9 that can be used to edit genes within organisms. CRISPR technology may include Class 1 CRISPR systems including type I (cas3), type III (cas10), and type IV and 12 subtypes. CRISPR technology may include Class 2 CRISPR systems including type II (cas9), type V (cas12), type VI (cas13), and 9 subtypes. In some embodiments, CRISPR technology may involve CRISPR-Cas design tools which are computer software platforms and bioinformatics tools used to facilitate the design of guide RNAs (gRNAs) for use with the CRISPR/Cas gene editing system. For example, CRISPR-Cas design tools may include: CRISPRon, CRISPRoff, Invitrogen TrueDesign Genome Editor, Breaking-Cas, Cas-OFFinder, CASTING, CRISPy, CCTop, CHOPCHOP, CRISPOR, sgRNA Designer, Synthego Design Tool, and the like. CRISPR technology may also be used as a diagnostic tool. For example, CRISPR-based diagnostics may be coupled to enzymatic processes, such as SHERLOCK-based Profiling of in vitro Transcription (SPRINT). SPRINT can be used to detect a variety of substances, such as metabolites in subject samples or contaminants in environmental samples, with high throughput or with portable point-of-care devices.

### C. Oocyte Rescue

i. Oocyte Scoring

At any stage of in vitro maturation or directly following in vitro maturation, an oocyte and/or granulosa cells may be appropriately frozen and stored for future analyses, experimentation, or for use in oocyte maturation. Oocytes may be scored with a scoring metric based on their morphology as determined by imagine analysis. In some embodiments, assignment of the scoring metric may include imaging the group cultures and analyzing the images of one or both of co-culture and no co-culture growth media-only control groups. In some embodiments, oocytes are scored and comparatively analyzed during any such stage of in vitro maturation. For example, group culture images may contain a pre-culture group COC image, a post-culture group COC image, and a post-culture denuded oocyte image. In some embodiments, oocytes subjected to scoring have never been frozen. In some embodiments oocytes subjected to scoring via image analysis may be thawed after storage by freezing. In some embodiments, oocytes subjected to scoring may be retrieved without in vitro maturation as described. In some embodiments, oocytes subjected to scoring may be cultured without described granulosa. In some embodiments, images may be sent to a qualified third party, such as an embryologist, developmental biologist, or other relevant skilled practitioner for scoring assignment.

In some embodiments of the methods described herein, oocytes may be assessed and subsequently classified by their maturation state according to the following criteria:
GV—presence of a germinal vesicle, typically containing a single nucleolus within the oocyte.
MI—absence of a germinal vesicle within the oocyte and absence of a polar body in the perivitelline space between the oocyte and the zona pellucida.
MII—absence of a germinal vesicle within the oocyte and presence of a polar body in the perivitelline space between the oocyte and the zona pellucida.

In some embodiments of the method, the scoring metric may include total oocyte scoring (TOS) as a function of analyzing the imaged group cultures via relevant microscopy or imaging analysis software. Methods and approaches of TOS have been described in the art (Lazzaroni-Tealdi et al., PLoS One 10:e0143632, 2015). Oocyte scoring may include metrics such as shape, size, ooplasm characteristics, structure of the perivitelline space (PVS), zona pellucida (ZP), polar body (PB) morphology, among other possible qualifiers. Total oocyte scoring on both pre and post culture oocyte images for generation of the TOS metric may be based on a scale system of −6 to +6.

Regarding oocyte shape, if oocyte morphology is poor (dark general oocyte coloration and/or ovoid shape), it may be assigned a value of −1; if it is almost normal (less dark general oocyte coloration and less ovoid shape), it may be assigned a value of 0; if it is judged to be normal, it may be assigned a value of +1. Regarding oocyte size: if oocyte size is defined as abnormally small or large, it may be assigned a value −1 if size is below 120 μm or greater 160 μm. If the size is almost normal, i.e., does not deviate from normal by more than 10 μm, a value of 0 may be assigned, and a value of +1 may be assigned if oocyte size is within normal range >130 μm and <150 μm. Regarding ooplasm characteristics, if the ooplasm is very granular and/or very vacuolated and/or demonstrates several inclusions, a value of −1 may assigned. If it is only slightly granular and/or demonstrates only few inclusions, a value of 0 may be assigned. Absence of granularity and inclusions may result in a +1 value. Regarding structure of the perivitelline space (PVS), the PVS may defined as −1 with an abnormally large PVS, an absent PVS or a very granular PVS. It may be assigned a value of 0 with a moderately enlarged PVS and/or small PVS and/or a less granular PVS. A value of +1 may be assigned to a normal size PVS with no granules. Regarding, zona pellucida (ZP), if ZPs is very thin or thick (<10 μm or >20 μm) the oocyte may be assigned a −1. If the ZP does not deviate from normal by more than 2 μm it may be assigned 0. A normal zona (>12 μm and <18 μm) may be assigned a +1. Regarding polar body (PB) morphology, PB morphology is defined as follows: Flat and/or multiple PBs or zero PBs, granular and/or either abnormally small or large PBs is designated as −1. PBs, judged as fair but not excellent may be designated as 0, and a designation of +1 may be given to PBs of normal size and shape. In some embodiments, MII oocytes PB score may not be aggregated into TOS.

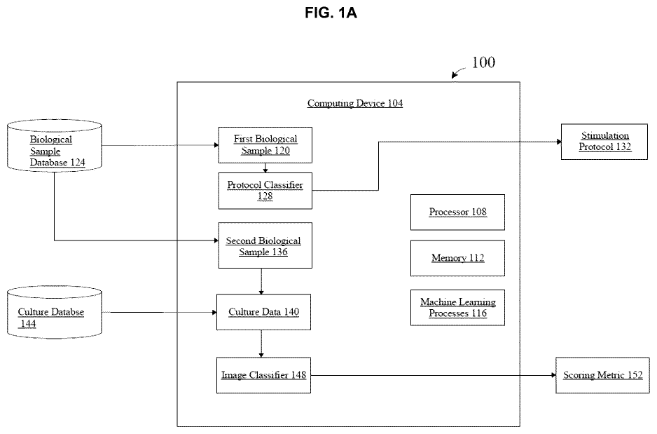

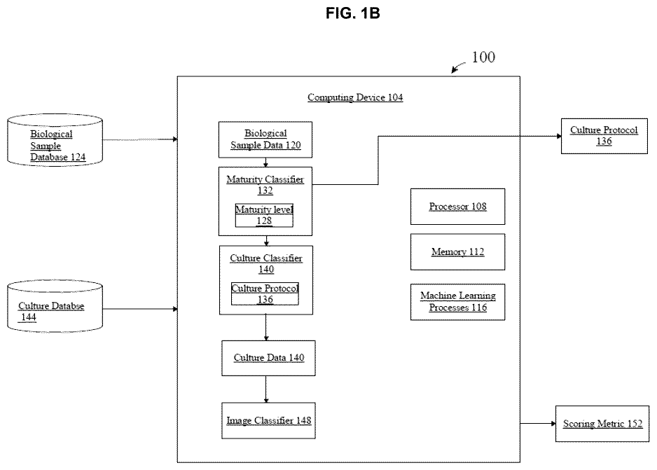

In some embodiments of the method, the scoring metric may include performing an outcome analysis as a function of the TOS as defined and exemplified in FIG. 1A. Parametric or non-parametric tests may be applied to determine the significance of findings during the analysis. Outcome analysis may be used to determine GV-stage to MII-stage oocyte maturation rate; GV-stage to MI-stage oocyte maturation rate; MI-stage to MII-stage oocyte maturation rate; Average Total Oocyte Score; Average Oocyte Shape; Average Oocyte Size; Average Ooplasm quality; Average PVS quality; Average ZP quality; Average Polar Body quality, and the like. In some embodiments these outcomes may reported as a as mean, median, and deviation. ii. In vitro fertilization and embryo culture In some embodiments of the methods, any one or more ova or oocytes as described herein may be evaluated for quality or maturation state, such as by the scoring metrics described herein, to determine their readiness for use in in vitro fertilization and embryo formation.

In some embodiments of the method, the ova or oocytes may be matured via in vitro maturation and subsequently utilized for IVF and/or ART as described herein. Any one or more oocytes may be utilized for intracytoplasmic sperm injection (ICSI). Following fertilization of the ovum by contact with one or more sperm cells, the subsequently formed zygote can be matured ex vivo so as to produce an embryo, such as a morula or blastula (e.g., a mammalian blastocyst), which can then be transferred to the uterus of a subject (e.g., a subject from which the oocyte was initially harvested) for implantation into the endometrium. Embryo transfers that can be performed using the methods described herein include fresh embryo transfers, in which the ovum or oocyte used for embryo generation is retrieved from the subject and the ensuing embryo is transferred to the subject during the same menstrual cycle. The embryo can alternatively be produced and cryopreserved for long-term storage prior to transfer to the subject.

iii. Omic Data Collection and Analyses of Oocytes, Cells, and Culture Media

In some embodiments of the method, the scoring metric may include an Omics-based analysis. For example, frozen cell lysates and cell culture media may be analyzed for bulk RNA-sequencing, whole genome bisulfite sequencing (WGBS), mass spectrometry-based proteomics and metabolomics. Cell culture media may be utilized for metabolomics analysis to determine changes in molecular content of media following co-culture compared to pre-culture media controls. This may be utilized to profile dynamic changes in paracrine signaling between granulosa cells and oocytes. The data gathered may then be aggregated for downstream analysis for determination of changes in epigenetic state, metabolite presence, and gene expression between different co-culture conditions and controls.

In some embodiments of the method, an omics-based analysis may include, genomics, proteomics, transcriptomics, pharmacogenomics, epigenomics, microbiomics, lipidomics, glycomics, transcriptomics culturomics, and/or any other omics one skilled in the art would understand as applicable. In some embodiments, after cultivation, an oocyte that has failed to mature, showing GV or MI characteristics, may be harvested for single cell RNA-sequencing, along with their associated granulosa cells from their culture. For this, oocytes and granulosa cells may be flash frozen and for library preparation. Of the oocytes that display MII oocyte development, half may be harvested for single cell RNA-sequencing along with their associated granulosa cells using the above flash freeze methods described throughout this disclosure. The remaining half of MII oocytes may be utilized for proteomic studies. The culture media for all conditions may additionally be flash frozen and utilized for metabolomics and proteomics to identify cholesterol metabolite levels and paracrine protein production. For example, frozen cell lysates and cell culture mediums may be analyzed for bulk RNA-sequencing, whole genome bisulfite sequencing (WGBS), mass spectrometry-based proteomics and metabolomics. Cell culture media may be utilized for metabolomics analysis to determine changes in molecular content of media following co-culture compared to pre-culture media controls to profile dynamic changes in paracrine signaling between granulosa cells and oocytes. As the media components are flash frozen, the sample is effectively quenched and amenable to metabolic assessment. The data gathered may then be aggregated for downstream analysis for determination of changes in epigenetic state, metabolite presence, and gene expression between different co-culture conditions and controls.

### III. Ex Vivo Compositions and Cell Culture Media

A. Granulosa Cells from hiPSCs

Specialized granulosa cells utilized in the methods described herein may be created from hiPSCs using transcription factor (TF)-directed protocols. In some embodiments, hiPSCs may be transformed with any one or more plasmids encoding one or more transcription factors. In some embodiments, hiPSCs may be transformed via electroporation, liposome-mediated transformation, viral-mediated gene transfer, among other cell transformation methodologies known in the art. In some embodiments, gene expression of desired transcription factors may be induced in a doxycycline-dependent manner. In some embodiments, transcription factors are constitutively expressed. In some embodiments, a plasmid or expression vector used for reprogramming hiPSCs may have a reporter gene such as a fluorescent protein. In some embodiments, hiPSCs may differentiate into granulosa with induced expression of transcription factors including FOXL2, NR5A1, GATA4, RUNX1, RUNX2, or a combination thereof. Reprogramming of hiPSCs to granulosa may be determined by genotyping methods known in the art. Reprogramming of hiPSCs to granulosa may be determined by protein expression using any one or more methods known in the art. Differentiation of hiPSCs to granulosa cells may be determined by relative expression of biomarkers typical of a granulosa cell type including AMHR2, CD82, FOXL2, FSHR, IGFBP7, KRT19, STAR, WNT4, or a combination thereof among other granulosa cell biomarkers known in the art. In some embodiments, reprogramming of hiPSCs to granulosa may be determined by production of growth factors and/or hormones including estradiol and progesterone that may adequately support in vitro maturation of retrieved oocyte via paracrine and juxtacrine cell signaling. In some embodiments, the resulting granulosa cells produce estradiol upon stimulation of androstenedione and FSH or forskolin. In some embodiments, the granulosa cells described herein may be produced in multiple batches. In some embodiments, the granulosa cells may be frozen and thawed prior to co-culture methods. In some embodiments, the granulosa cells were freshly differentiated prior to in vitro maturation method. In some embodiments, the granulosa cells may be seeded and equilibrated for 2-8 hours (e.g., 2-3 hours, 2-4 hours, 3-4 hours, 4-6 hours, 5-7 hours, 6-8 hours; e.g., 2 hours, 3 hours, 4 hours, 5 hours, 6 hours, 7 hours, 8 hours) before the addition of oocytes for in vitro maturation.

In some embodiments, a subject may donate hiPSCs. hiPSCs donation may follow an oocyte retrieval process as discussed above. A subject participating in hiPSCs donation may be different, or the same, from the subject from which the oocyte was retrieved. In some embodiments, a hiPSC donor may undergo a stimulation protocol as disclosed above. In some embodiments, hiPSCs, granulosa cells, cumulus cells, oocytes, GV-stage oocytes, MI-stage oocytes, MII-stage oocytes and all other types of cells described through this disclosure may be lysed, extracted for genomic material and flash frozen as the final step in the culture process. For example, cells may undergo enzymatic cell lysis using enzymes such as lysozyme, lysostaphin, zymolase, cellulose, protease or glycanase, and the like. Other lysis methods may be applied such as chemical lysis, detergent lysis, alkaline lysis, mechanical lysis, thermal lysis, acoustic lysis, physical lysis, non-mechanical lysis and other lysis methods known in the art. In some embodiments, culture media may be flash frozen. Freezing methods may include using a cryoprotective agent such as dimethyl sulfoxide and/or any other freezing method known in the art.

b. Transgenic Granulosa Cells

Specialized transgenic granulosa cells may be produced using Clustered Regularly Interspaced Short Palindromic Repeats (CRISPR) technology. “CRISPR” is programmable technology that targets specific stretches of genetic code to edit DNA at precise locations. CRISPR technology may include CRISPR-CAS 9. Cas9 (or “CRISPR-associated protein 9”) is an enzyme that uses CRISPR sequences as a guide to recognize and cleave specific strands of DNA that are complementary to the CRISPR sequence. Cas9 enzymes together with CRISPR sequences form the basis of a technology known as CRISPR-Cas9 that can be used to edit genes within organisms. CRISPR technology may include Class 1 CRISPR systems including type I (cas3), type III (cas10), and type IV and 12 subtypes. CRISPR technology may include Class 2 CRISPR systems including type II (cas9), type V (cas12), type VI (cas13), and 9 subtypes. In some embodiments, CRISPR technology may involve CRISPR-Cas design tools which are computer software platforms and bioinformatics tools used to facilitate the design of guide RNAs (gRNAs) for use with the CRISPR/Cas gene editing system. For example, CRISPR-Cas design tools may include: CRISPRon, CRISPRoff, Invitrogen TrueDesign Genome Editor, Breaking-Cas, Cas-OFFinder, CASTING, CRISPy, CCTop, CHOPCHOP, CRISPOR, sgRNA Designer, Synthego Design Tool, and the like. CRISPR technology may also be used as a diagnostic tool. For example, CRISPR-based diagnostics may be coupled to enzymatic processes, such as SHERLOCK-based Profiling of in vitro Transcription (SPRINT). SPRINT can be used to detect a variety of substances, such as metabolites in subject samples or contaminants in environmental samples, with high throughput or with portable point-of-care devices.

### C. Cell Culture Media

Granulosa cells, such as granulosa cells derived from iPSCs (e.g., hiPSCs) or transgenic granulosa cells (as described above), may be provided as a composition further containing a cell culture media (e.g., IVF, IVM, (e.g., MediCult IVM media), or LAG media). The cell culture media may include human serum albumin (e.g., at about 5-15 mg/mL, e.g., 10 mg/mL), FSH (e.g., at about 70-80 mIU/mL, e.g., 75 mIU/mL), hCG (e.g., at about 95-105 mIU/mL, e.g., 100 mIU/mL), Androstenedione (e.g., at about 495-505 ng/mL, e.g., 500 ng/mL), Doxycycline (e.g., 0.5-1.5 μg/mL, e.g., 1 μg/mL) and other compounds such as hyaluronidase and/or dPBS.

### IV. Apparatuses and Associated Methods for Oocyte Maturation

The present disclosure presents apparatuses for use in assisted reproductive technology (e.g., IVF). FIG. 1 depicts two exemplary apparatuses (e.g., see FIG. 1A and FIG. 1B). Referring to FIG. 1A, an exemplary embodiment of an apparatus 100 for inducing human oocyte maturation in vitro is illustrated. Referring to FIG. 1B, an exemplary embodiment of an apparatus 100 for aiding in oocyte rescue in vitro post stimulation is illustrated.

### A. Computing Devices

In an apparatus of the disclosure (e.g., FIG. 1A and FIG. 1B), the apparatus 100 includes a computing device 104. Computing device 104 includes a processor 108 and a memory 112 communicatively connected to the processor 108, wherein memory 112 contains instructions configuring processor 108 to carry out the linking process. Processor 108 and memory 112 is contained in a computing device 104. Computing device 104 may include any computing device as described in this disclosure, including without limitation a microcontroller, microprocessor, digital signal processor (DSP) and/or system on a chip (SoC) as described in this disclosure. Computing device may include, be included in, and/or communicate with a mobile device such as a mobile telephone or smartphone. Computing device 104 may include a single computing device operating independently, or may include two or more computing device operating in concert, in parallel, sequentially or the like; two or more computing devices may be included together in a single computing device or in two or more computing devices. Computing device 104 may interface or communicate with one or more additional devices as described below in further detail via a network interface device. Network interface device may be utilized for connecting computing device 104 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. Computing device 104 may include but is not limited to, for example, a computing device or cluster of computing devices in a first location and a second computing device or cluster of computing devices in a second location. Computing device 104 may include one or more computing devices dedicated to data storage, security, distribution of traffic for load balancing, and the like. Computing device 104 may distribute one or more computing tasks as described below across a plurality of computing devices of computing device, which may operate in parallel, in series, redundantly, or in any other manner used for distribution of tasks or memory between computing devices. Computing device 104 may be implemented using a “shared nothing” architecture in which data is cached at the worker, in an embodiment, this may enable scalability of system 100 and/or computing device.

With continued reference to apparatuses of the disclosure (e.g., FIG. 1A and FIG. 1B), computing device 104 may be designed and/or configured to perform any method, method step, or sequence of method steps in any embodiment described in this disclosure, in any order and with any degree of repetition. For instance, computing device 104 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Computing device 104 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

With continued reference to apparatuses of the disclosure (e.g., FIG. 1A and FIG. 1B), additionally, computing device 104 may perform determinations, classification, and/or analysis steps, methods, processes, or the like as described in this disclosure using machine learning processes 116. A “machine learning process,” as used in this disclosure, is a process that automatedly uses a body of data known as “training data” and/or a “training set” to generate an algorithm that will be performed by a computing device/module to produce outputs given data provided as inputs; this is in contrast to a non-machine learning software program where the commands to be executed are determined in advance by a user and written in a programming language. Machine-learning process 116 may utilize supervised, unsupervised, lazy-learning processes and/or neural networks.

i. Oocyte Maturation

With specific reference to the apparatus of FIG. 1A, computing device 104 is configured to aid in human oocyte maturation in vitro by promoting rapid and efficient oocyte maturation in a manner that reinforces oocyte health and developmental competence. Computing device 104 is configured to receive first biological sample data from a first biological sample 120 relating to a user. As used in this disclosure, “biological sample data” is data that provides a characterization of the biological, genetic, biochemical and/or physiological properties, compositions, or activities of biological samples. A “biological sample” is any information relating to a user. A biological sample may include laboratory specimen held by a biorepository for research. In some embodiments, first biological sample 120 may include bodily fluids including blood, saliva, urine, semen (seminal fluid), vaginal secretions, cerebrospinal fluid (CSF), synovial fluid, pleural fluid (pleural lavage), pericardial fluid, peritoneal fluid, amniotic fluid, saliva, nasal fluid, optic fluid, gastric fluid, breast milk, cell culture supernatants, and the like. A biological sample may include a medical diagnosis, user input describing how a user is feeling and/or a symptomatic complaint, information collected from a wearable device pertaining to a user and the like. For example, a biological sample may include information obtained from a visit with a medical professional such as a health history. In yet another non-limiting example, a biological sample may include information such as data collected from a wearable device worn by a user and designed to collect information relating to a user's sleep patterns, exercise patterns, and the like. In an embodiment, first biological sample 120 may be collected at a particular date and/or time of a user's menstrual cycle. For instance and without limitation, first biological sample 120 may be collected on the second day of a user's menstrual cycle to evaluate one or more hormone levels such as E2, FSH, LH, P4, and/or AMH. First biological sample 120 may be utilized to determine one or more markers of a user's overall health including but not limited to ovarian reserve health and/or circulating hormone levels. This information may be utilized for example by a health care professional to monitor cycle progression and inform protocol and/or drug selection.

As used in this disclosure, a “user” is a living organism such as a human being, plant, animal, and all other organisms composed of cells. In some embodiments, the biological sample may be extracted from the user through an extraction device. An “extraction device” is a device and/or tool capable of obtaining, recording and/or ascertaining a measurement associated with a sample. The extraction device may include a needle, syringe, vial, lancet, Evacuated Collection Tubes (ECT), tourniquet, vacuum extraction tube systems, any combination thereof and the like. For example, the extraction device may comprise a butterfly needle set. Computing device 104 may receive the biological sample in the form of data uploaded to the memory. Data may include measurements, for example, of serum calcium, phosphate, electrolytes, blood urea nitrogen and creatinine, uric acid, and the like.

Still referring to FIG. 1A, computing device 104 may receive first biological sample data from a biological sample database 124. A “biological sample database” is a database containing all data related biological samples of users containing analytic information.

ii. Oocyte Rescue

With specific reference to the apparatus of FIG. 1B, computing device 104 is configured to aid in oocyte rescue in vitro post stimulation. An “oocyte rescue” is the process of maturing immature oocyte cells in vitro that are typically disregarded in standard in vitro maturation procedures. An “oocyte,” as used in this disclosure, is a reproductive cell originating in an ovary. Post simulation may refer to standard in vitro fertilization IVF stimulation protocols performed on a user. As used in this disclosure, a “stimulation protocol” is a medication injection process spanning over a specified period of time to induce ovaries in producing one or more oocytes. As used in this disclosure, a “user” is a living organism such as a human being, plant, animal, and all other organisms composed of cells. In some embodiments, post stimulation may refer to a minimal stimulation protocol. As used in this disclosure, a “minimal stimulation protocol” is a stimulation process spanning over a shortened period of time, compared to average in vitro fertilization (IVF) stimulation protocols, to aid in inducing an ovary to produce an egg. Typically, the average span of time for a stimulation protocol using standard IVF is around 8-14 days. The minimal stimulation protocol may induce the release of a cell in 2-6 days, which is a shortened period of time compared to 8-14 days. The average time for performing minimal stimulation protocol 132 may be 3 days. The max time may be 6 days and the minimal amount of time may be 2 days.

Still referring to FIG. 1B, computing device 104 is configured to receive biological sample data 120 from a biological sample relating to a user, including at least an oocyte. As used in this disclosure, “biological sample data” is data that provides a characterization of the biological, genetic, biochemical and/or physiological properties, compositions, or activities of biological samples. A “biological sample,” as used in this disclosure, is a biological laboratory specimen obtained from a subject (e.g., a blood sample or other bodily fluid sample). In some embodiments, an oocyte may be an immature oocyte. An “immature oocyte” as used in this disclosure is a one or more immature reproductive cells originating in the ovaries.

In some embodiments, an immature oocyte may be an oocyte including GV-stage and/or MI-stage oocytes. In some embodiments, an immature oocyte may be a plurality of oocytes. An immature oocyte may be immature cumulus-oocyte-complexes (COCs) taken from the mother. As used in this disclosure, a “cumulus-oocyte-complex” is an oocyte surrounded by specialized granulosa cells. As used in this disclosure, a“specialized granulosa cell” is a cumulus cell surrounding the oocyte to ensure healthy oocyte and embryo development. In some embodiments, the immature oocyte may contain an oocyte wherein the specialized granulosa cell is added to mature the oocyte in a cell culture and thus create a COC. In some embodiments, the biological sample may include bodily fluids including blood, saliva, urine, semen (seminal fluid), vaginal secretions, cerebrospinal fluid (CSF), synovial fluid, pleural fluid (pleural lavage), pericardial fluid, peritoneal fluid, amniotic fluid, nasal fluid, optic fluid, gastric fluid, breast milk, cell culture supernatants, and the like.

Still referring to FIG. 1B, computing device 104 may receive biological sample data 120 from a biological sample relating to a user post stimulation from a biological sample database 124. As used in this disclosure a “biological sample database” is a database containing all data related biological samples of users containing analytic information. In some embodiments, biological sample database 124 may contain a stimulation protocol index. A “stimulation protocol index,” as used in this disclosure, is data structure correlating user information regarding the completion of a stimulation protocol such as: user age, user BMI, number of COCs retrieved, number of mature MII-stage oocytes, number of immature MI oocytes, number of immature GV oocytes, AMH Levels (μ/L), antral follicle count (AFC) at last ultrasound, user oocyte retrieval day E2 Levels (ng/L), user oocyte retrieval day P4 Levels (ng/L), user oocyte retrieval day LH (IU/L), user oocyte retrieval day FSH (IU/L), Days of stimulation, gonadotropin used, and total injected dose (IU).

iii. Biological Sample Database

In some embodiments of an apparatus (e.g., of FIG. 1A and FIG. 1B), biological sample database 124 may contain a systemic hormone index. A “systemic hormone index,” as used in this disclosure, is data structure correlating medical knowledge regarding systemic hormone therapy. For example, a systemic hormone index may include side effects and risks, proper methods of administering hormones, correlations between E2, LH, FSH, and/or P4 deficiency and systemic hormones and the like. In some embodiments, biological sample database 124 may be communicatively connected to computing device 104. As used in this disclosure, “communicatively connected” means connected by way of a connection, attachment, or linkage between two or more relata which allows for reception and/or transmittance of information therebetween. For example, and without limitation, this connection may be wired or wireless, direct, or indirect, and between two or more components, circuits, devices, systems, and the like, which allows for reception and/or transmittance of data and/or signal(s) therebetween. Data and/or signals therebetween may include, without limitation, electrical, electromagnetic, magnetic, video, audio, radio, and microwave data and/or signals, combinations thereof, and the like, among others. A communicative connection may be achieved, for example and without limitation, through wired or wireless electronic, digital, or analog, communication, either directly or by way of one or more intervening devices or components. Further, communicative connection may include electrically coupling or connecting at least an output of one device, component, or circuit to at least an input of another device, component, or circuit. For example, and without limitation, via a bus or other facility for intercommunication between elements of a computing device. Communicative connecting may also include indirect connections via, for example and without limitation, wireless connection, radio communication, low power wide area network, optical communication, magnetic, capacitive, or optical coupling, and the like. In some instances, the terminology “communicatively coupled” may be used in place of communicatively connected in this disclosure.

A biological sample database 124, and all other databases described throughout this disclosure, may be implemented, without limitation, as a relational database, a key-value retrieval database such as a NOSQL database, or any other format or structure for use as a database that a person skilled in the art would recognize as suitable upon review of the entirety of this disclosure. Biological sample database 124 may alternatively or additionally be implemented using a distributed data storage protocol and/or data structure, such as a distributed hash table or the like. Biological sample database 124 may include a plurality of data entries and/or records. Data entries in a database may be flagged with or linked to one or more additional elements of information, which may be reflected in data entry cells and/or in linked tables such as tables related by one or more indices in a relational database. Persons skilled in the art, upon reviewing the entirety of this disclosure, will be aware of various ways in which data entries in a database may store, retrieve, organize, and/or reflect data and/or records as used herein, as well as categories and/or populations of data consistently with this disclosure.

iv. Stimulation Protocol

In an apparatus of the disclosure (e.g., see FIG. 1A), computing device 104 is configured to assign a user to a stimulation protocol 132 as a function of first biological sample 120. In some embodiments, the stimulation protocol 132 may be assigned based on a measured hormone level of the biological sample. A “stimulation protocol” is a medication (e.g., triggering agent) injection process spanning over a specified period of time (e.g., the follicular triggering period) to induce ovaries in producing one or more oocytes.

As used in this disclosure, a “measured hormone level” is a quantitative value representing a level of one or more hormones of a user. The measured hormone level of the biological sample may include estradiol (E2), luteinizing hormone (LH), follicle-stimulating hormone (FSH), progesterone (P4), estrone (E1), estriol (E3), testosterone, androgens, dehydroepiandrosterone (DHEA), triiodothyronine (T3), tetraiodothyronine (T4), calcitonin, melatonin, insulin, cortisol, human growth hormone (HGH), adrenaline levels and the like. In some embodiments, the measurement of hormone levels may be based on blood analysis a of the biological sample. For example, blood analysis may include plasma hormone analysis techniques. In some embodiments, measurement of hormone levels may be based on saliva hormone testing techniques. Measurement of hormone levels may be based on other forms of analysis such as hair, urine, and any other form of biological samples described throughout this disclosure.

In an embodiment (e.g., see FIG. 1A), selection of a stimulation protocol may be done utilizing information obtained from an ultrasound. An “ultrasound,” as used in this disclosure, is any procedure that utilizes sound waves to generate one or more images of a user's body. For example, an ultrasound may be utilized to obtain an image of a subject's reproductive organs and/or tissues. In an embodiment, an ultrasound may be performed at a particular time of a subject's menstrual cycle. For example, a subject may receive an ultrasound on day 2 of her cycle and this may be utilized to determine follicle size and/or follicle count. Selection of a stimulation protocol and/or adjustment to a stimulation protocol may be made utilizing this information. For example, a subject with an ultrasound that shows polycystic ovarian syndrome (PCOS) may have a dose adjustment made to one or more medications received and/or utilized during a stimulation protocol. In addition, the length of her stimulation protocol may be modified based on her PCOS diagnosis. In an embodiment, an ultrasound may be repeated one or more times throughout a subject's stimulation protocol, and information obtained may be utilized to adjust her stimulation protocol in real time.

In addition, a subject's contraception usage may affect assignment of a stimulation protocol. “Contraception,” as used in this disclosure, is any method and/or device utilized to prevent pregnancy as a consequence of sexual intercourse. This may include but is not limited to any medication, technique, device, and/or birth control utilized by a subject. A subject's use of contraception or not may aid in determining at what point in the subject's menstrual cycle she should begin her stimulation protocol. For instance and without limitation, a subject who is not using any form of contraception may begin her stimulation protocol with recombinant follicle stimulating hormone (rFSH) on the second day of her menstrual cycle. In yet another non-limiting example, a subject who is using contraception may begin her stimulation protocol with rFSH 5 days after consuming her last oral contraception pill. In an embodiment, rFSH stimulation may be utilized for 2-3 days, depending on a subject's tolerance, follicle size, and/or growth dynamics. After this 2-3 day window, a coasting period of 1-2 days may be utilized to monitor follicle size and allow for further follicle maturation and development. A “coasting period,” as used in this disclosure, is any period of time when a medication used throughout a stimulation protocol is not administered and/or consumed. A coasting period may last for example for 1 day, 2 days, 3 days, and the like. During a coasting period, a subject may continue to receive one or more ultrasounds to monitor her progression.

Once a follicle size has reached anywhere from between 8-10 mm, a subject may be triggered with a dose of human chorionic gonadotropin (hCG). In an embodiment, a double hCG injection may be utilized, to induce follicle maturation to prepare one or more follicles for retrieval. A blood test for one or more hormone levels such as E2, P4, and LH may be performed on trigger day of double dose of human chorionic gonadotropin (hCG) injection to monitor hormone levels. After the day of the double dose of hCG, one or more hormone levels may be measured such as for example a blood test to determine and examine doses of E2, P4, and LH.

Approximately 24-48 hours after the dose of hCG that is administered, a subject may undergo an oocyte retrieval. On the day of oocyte retrieval, a blood test for one or more hormone levels such as E2, LH, and/or P4 may be performed to ensure quality metrics, hormone levels are within range, and/or that hCG dose was ingested. The assigned stimulation protocol 132 may include a minimal stimulation protocol configured to trigger the release of a cell in the span of 3 days. A “minimal stimulation protocol” is a stimulation process spanning over a shortened period of time, compared to average in vitro fertilization (IVF) stimulation protocols, to aid in inducing an ovary to produce an egg. Typically, the average span of time for a stimulation protocol using standard IVF is around 8-14 days. The minimal stimulation protocol may induce the release of a cell in 2-6 days, which is a shortened period of time compared to 8-14 days. The average time for performing minimal stimulation protocol 132 may be 3 days. The max time may be 6 days and the minimal amount of time may be 2 days. In an embodiment, the minimal stimulation protocol may include selecting a first triggering agent (e.g., a follicular triggering agent) as a function of the first biological sample 120 and selecting a second triggering agent (e.g., a follicular triggering agent) as a function of a follicle measurement, which is disclosed in greater detail below. A “follicle measurement” as used in this disclosure, is any measurement of an ovarian follicle. A follicle may include any sac found in an ovary that contains an unfertilized egg. A follicle measurement may be obtained using any methodology as described herein, including for example an ultrasound, a manual measurement, an automated measurement and the like.

A “triggering agent” is a chemical that triggers cell generation in the ovaries. A triggering agent (e.g., a follicular triggering agent) may include any substance including any non-prescription and/or prescription product. A triggering agent (e.g., a follicular triggering agent) may include for example, Lupron as produced by Abbott Laboratories, headquartered in North Chicago, IL; Ganirelix as produced by Ferring Pharmaceuticals, headquartered in Saint-Prex, Switzerland; Cetrotide as produced by Merck Global, headquartered in Whitehouse Station, Readington Township, NJ; Gonal-F as produced by Merck Global, Follistim as produced by Merck Global; Bravelle as produced by Ferring Pharmaceuticals, headquartered in Saint-Prex; Switzerland, Clomid as produced by Patheon Pharmaceuticals Inc., headquartered in Waltham, MA; Serephene as produced by Teva, headquartered in Tel Aviv-Yafo, Israel; Glucophage as produced by Merck Global; Fortamet as produced by Mylan, headquartered in Canonsburg, PA; Pregnyl as produced by Schering Plough, headquartered in Kenilworth, NJ; Novarel as produced by Ferring Laboratories, headquartered in Parsippany, NJ; Repronex as produced by Ferring Pharmaceuticals, Inc.; Factrel as produced by Zoetis Canada Inc., headquartered in Kirkland, Canada, Menopur as produced by Ferring Pharmaceuticals, and other drugs that induce cell generation in ovaries that one skilled in the art would understand as applicable. A triggering agent (e.g., a follicular triggering agent) may include human serum albumin, FSH, hCG, androstenedione, and doxycycline. Computing device 104 may assign the triggering agents used based on the measured hormone levels of first biological sample 120. In some embodiments, computing device 104 may use a machine learning process to generate and/or train a machine-learning model including a classifier. In an embodiment, and continuing to refer to FIG. 1A, a machine-learning model may be utilized to assign a user to a particular stimulation protocol as a function of first biological sample 120.

v. Classifiers

A “classifier,” as used in this disclosure is a machine-learning model, such as a mathematical model, neural net, or program generated by a machine learning algorithm known as a “classification algorithm,” as described in further detail below, that sort inputs into categories or bins of data, outputting the categories or bins of data and/or labels associated therewith. A classifier may be configured to output at least a datum that labels or otherwise identifies a set of data that are clustered together, found to be close under a distance metric as described below, or the like.

In an apparatus of the disclosure (e.g., FIG. 1A and FIG. 1B), classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. Computing device 104 may be configured to generate a classifier using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A) P(A)+P(B), where P(A/B) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Computing device 104 may then calculate a likelihood table by calculating probabilities of different data entries and classification labels. Computing device 104 may utilize a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

With continued reference to FIG. 1A and FIG. 1B, computing device 104 may be configured to generate a classifier using a K-nearest neighbors (KNN) algorithm. A “K-nearest neighbors algorithm” as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

With continued reference to FIG. 1A and FIG. 1B, generating k-nearest neighbors algorithm may generate a first vector output containing a data entry cluster, generating a second vector output containing an input data, and calculate the distance between the first vector output and the second vector output using any suitable norm such as cosine similarity, Euclidean distance measurement, or the like. Each vector output may be represented, without limitation, as an n-tuple of values, where n is at least two values. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions. Any vectors as described herein may be scaled, such that each vector represents each attribute along an equivalent scale of values. Each vector may be “normalized,” or divided by a “length” attribute, such as a length attribute I as derived using a Pythagorean norm:

$$l = \sqrt{{\sum }_{i = 0}^{n} {a}_{i}^{2}} ,$$

where aᵢ is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values.

In reference to the apparatus of FIG. 1A, a computing device 104 and/or another device may generate protocol classifier 128 using a classification algorithm. A “protocol classifier” is a classifier trained to intake biological samples relating to a user and output/assign stimulation protocol 132 to the related user based on training data received. Training data may consist of inputs and/or outs containing systemic hormone index data, feedback from past stimulation protocol 132 assignments, and any other data described throughout this disclosure. Training data may be received from biological sample database 124. In some embodiments, training data may include a plurality of data entries containing biological samples correlated to outputs containing assigned stimulation protocols. In some embodiments, training data may include inputs such as assigned stimulation protocols correlated to outputs such as pregnancy success rate or scoring metrics as described throughout this disclosure. In some embodiments, training data may include correlations between a stimulation protocol and the correlated side effects. In some embodiments, training data may include methods and procedures to prevent hyperstimulation of the ovaries by the triggering agent. For example, training data may include the number of injections a user may receive containing a specific triggering agent (e.g., a follicular triggering agent) at a plurality of doses before hyperstimulation occurs.

In any apparatus of the disclosure (e.g., FIG. 1A and FIG. 1B), computing device 104 may train any classifier or other machine-learning model using training data. In some embodiments, training data may include correlations between biological sample data and or biological samples extracted from users to maturity levels, scores, or other numerical and/or quantitative fields related to the oocyte, for instance and without limitation in the form of training examples. Training examples may be entered by one or more experts. An “expert” as used in this disclosure is a person or organization skilled in the art. Expert knowledge may be retrieved from the feedback index in biological sample database 124.

A biological sample containing an oocyte may be retrieved from a user post simulation by a medical professional, such as a doctor inserting an extraction device into the follicle containing an egg and extracting the egg and surrounding fluid. An “extraction device” is a device and/or tool capable of obtaining, recording and/or ascertaining a measurement associated with a sample. The extraction device may include a needle, syringe, vial, lancet, Evacuated Collection Tubes (ECT), tourniquet, vacuum extraction tube systems, any combination thereof and the like. For example, the extraction device may comprise a butterfly needle set. Oocyte retrieval may include retrieval of immature oocytes, mature oocytes, COCs, and any other type of cell involved in reproduction found in the ovaries.

In reference to the apparatus of FIG. 1B, a computing device 104 is configured to determine a maturity level 128 of the at least an oocyte. A “maturity level,” as used in this disclosure, is a datum representing an assessment of the oocyte stage of oogenesis; maturity level may include a quantitative element and/or field such as a number of days, hours, or the like of maturity, a stage of maturity, and/or a score representing a degree of maturity. In some embodiments, maturity level 128 may be an assessment of the oocyte maturation stage of oogenesis. Oocyte maturation refers to a release of meiotic arrest that allows oocytes to advance from prophase I to metaphase II of meiosis. Determining maturity level 128 of the oocyte may include denuding the oocyte. Oocyte denudation refers to the removal of the somatic cell layers that surround the oocytes. For example, a COC may be denuded to remove the layer of granulosa cells surrounding the oocyte in order to determine the nuclear maturity of the oocyte. Oocyte denudation may include enzymatic and mechanical methods with the help of hyaluronidase and sterile glass pipettes as described further below.

Still referring to the apparatus of FIG. 1B, in some embodiments, determining maturity level 128 may include determination of the maturity level 128 using a machine-learning process 116. For instance, and without limitation, a machine learning process 116 may be used to generate and train a machine learning model containing a classifier; the classifier may classify the oocyte to a maturity level as a function of biological sample data.

Still referring to the apparatus of FIG. 1B, computing device 104 and/or another device may generate maturity classifier 132 using a classification algorithm. As used in this disclosure, maturity classifier” is a classifier trained to intake biological sample data 120 and output a maturity level 128. Additionally or alternatively, maturity classifier 132 may be trained to intake oocyte denudation data to output a maturity level 128. As used in this disclosure, “oocyte denudation data” is data that provides a characterization of the biological, genetic, biochemical and/or physiological properties, compositions, or activities of a denuded oocyte. Computing device 104 may receive oocyte denudation data from biological sample database. In some embodiments, computing device 104 may train maturity classifier 132 with training data including correlations between an oocyte to a maturity level, for instance and without limitation in the form of training examples. Training examples may be derived from a maturity level index, and feedback index contained in a biological sample database. As used in this disclosure, a “maturity level index” is data structure correlating biological knowledge relating to the stages of oocyte oogenesis, such as the stages of oocyte maturation. As used in this disclosure, feedback index” is a data structure correlating past maturity level 128 assessments performed by the computing device and communication from a third party. As used in this disclosure, a “third party” is a qualified person or organization, such as an embryologist, statistician, and the like. In some embodiments, maturity classifier 132 training data may be received from biological sample database. For example, biological sample data 120 may contain data relating to denuded immature COCs, and with the training data, determine, as maturity level 128, the oocytes resulting from denudation are GV and or MI oocytes. In some embodiments, a machine learning model containing a classifier trained to classify the oocyte to a maturity level as a function of biological sample data may also calculate a maturity level, a score, or other numerical and/or quantitative field related to the oocyte using machine learning processes 116.

vi. Second Biological Samples

Apparatuses described herein (e.g., see FIG. 1A) may be configured to receive a second biological sample. In FIG. 1A, for example, computing device 104 is configured to receive second biological sample data from second biological sample 136 relating to the user, wherein second biological sample 136 includes at least an oocyte. An “oocyte,” as used in this disclosure, is a reproductive cell originating in an ovary. An oocyte may include but is not limited to an immature oocyte, a mature oocyte, a group of one or more oocytes, a group of one or more cells, a cumulus oocyte complex and the like. A “cumulus oocyte complex,” as used in this disclosure, is an oocyte containing one or more surrounding cumulus cells. A COC may contain an immature oocyte. A COC may contain a mature oocyte. An “immature oocyte” as used in this disclosure is one or more immature reproductive cells originating in the ovaries. In some embodiments, an immature oocyte may be an oocyte including but not limited to germinal vesicle (GV) and Metaphase 1 (M1) oocytes, as described further below. In some embodiments, an immature oocyte may be a plurality of oocytes. An immature oocyte may be immature cumulus-oocyte-complexes (COCs) taken from a subject. A “mature oocycte” as used in this disclosure, is one or more mature reproductive cells originating in the ovaries. Once retrieved, a COC may rest for 2-3 hours to allow for equilibration to in vitro conditions. In an embodiment, an oocyte may be combined with a specialized granulosa cell. A “specialized granulosa cell” is a cumulus cell capable of surrounding the oocyte to ensure healthy oocyte and embryo development. In some embodiments, the immature oocyte may contain an oocyte wherein the specialized granulosa cell is added to mature the oocyte in a cell culture and thus create a COC after extraction of second biological sample 136. Second biological sample 136 may include bodily fluids as disclosed above. In an embodiment, one or more specialized granulosa cells may be thawed during resting period of one or more COCs. In an embodiment, anywhere from between 50,000-100,000 specialized granulosa cells may be combined with a COC during culturing. In an embodiment, thawed specialized granulosa cells may be placed into a culture medium prior to COC retrieval, including anywhere form 24-120 hours beforehand. A COC may be transferred into culture medium containing thawed specialized granulosa cells to form a group culture as described below in more detail. In an embodiment, a group culture may be culture in an incubator ranging in time from anywhere from 12-48 hours. Computing device 104 may receive second biological sample data form biological sample database 124 as described above. Second biological sample 136 may be extracted using an extraction device and received as disclosed above. In some embodiments, computing device 104 may record the measured hormone level of second biological sample 136 using methods as disclosed above. Specialized granulosa cells may be produced using Clustered Regularly Interspaced Short Palindromic Repeats (CRISPR) technology. “CRISPR” is programmable technology that targets specific stretches of genetic code to edit DNA at precise locations. CRISPR technology may include CRISPR-CAS 9. Cas9 (or “CRISPR-associated protein 9”) is an enzyme that uses CRISPR sequences as a guide to recognize and cleave specific strands of DNA that are complementary to the CRISPR sequence. Cas9 enzymes together with CRISPR sequences form the basis of a technology known as CRISPR-Cas9 that can be used to edit genes within organisms. CRISPR technology may include Class 1 CRISPR systems including type I (cas3), type III (cas10), and type IV and 12 subtypes. CRISPR technology may include Class 2 CRISPR systems including type II (cas9), type V (cas12), type VI (cas13), and 9 subtypes. In some embodiments, CRISPR technology may involve CRISPR-Cas design tools which are computer software platforms and bioinformatics tools used to facilitate the design of guide RNAs (gRNAs) for use with the CRISPR/Cas gene editing system. For example, CRISPR-Cas design tools may include: CRISPRon, CRISPRoff, Invitrogen TrueDesign Genome Editor, Breaking-Cas, Cas-OFFinder, CASTING, CRISPy, CCTop, CHOPCHOP, CRISPOR, sgRNA Designer, Synthego Design Tool, and the like. CRISPR technology may also be used as a diagnostic tool. For example, CRISPR-based diagnostics may be coupled to enzymatic processes, such as SHERLOCK-based Profiling of in vitro Transcription (SPRINT). SPRINT can be used to detect a variety of substances, such as metabolites in subject samples or contaminants in environmental samples, with high throughput or with portable point-of-care devices.

In some embodiments regarding the culture of second biological sample 136, cell culture media may include LAG media. For example, LAG media may be used for the incubation of COCs post-retrieval from stimulation protocol 132. Package size may be a 10 mL vial. Storage may be at 2-8° C. away from light up to one month. Media equilibration may be 18 to 24 hours pre-culture, include a seed 100 ul droplet and placed into 37° C. incubator with 6% O₂ and proper CO₂. In some embodiments, cell culture media may include IVM media (e.g., from 1 mL to 100 mL of media may be used per co-culture, such as 1 mL, 5 mL, 10 mL, 20 mL, 30 mL, 40 mL, 50 mL, 60 mL, 70 mL, 80 mL 90 mL, or 100 mL). For example, a modified-MediCult IVM media may be used a baseline control during the culturing process. Package size may be a 10 mL vial. Storage may be at 2-8° C. away from light up to one month. In some embodiments, the cell culture mediums may include metabolites. For example, the modified-MediCult IVM media may include human serum albumin, FSH, hCG, Androstenedione, Doxycycline and other compounds. Other cell culture material and equipment may include: liquid nitrogen, hyaluronidase, dPBS, IVF-Qualified mineral oil, universal GPS dishes, G-NOPS plus media, micropipettes, stripper pipettors, camera-equipped inverted ICSI Microscope, Dry Inject Tabletop incubators, saturated humidity incubators, EmbryoScope, microcentrifuge, refrigerator, −20° C. freezer to 100° C. freezer, liquid nitrogen storage dewer, 35 mm dishes for denuding, stripper pipette tips, and other components one skilled in the art would understand to be included in the cell culture process.

Still referring to the apparatus of FIG. 1A, in some embodiments, culturing second biological sample 136 may include culturing the immature Cumulus-Oocyte complexes (COCs) in a group culture. A “group culture” is an extracted COC combined with one or more additional cells. An additional cell may include any cell grown together with an extracted COC. An additional cell may include a specialized granulosa cell. In an embodiment, a group culture may be cultured and/or incubated for a particular length of time, such as from between 12-120 hours. For example, group culturing may include culturing the Cumulus-Oocyte complexes with a granulosa co-culture as described further below. A “co-culture” is a cell cultivation set-up, in which two or more different populations of cells are grown with some degree of contact between them. In some embodiments, group culturing may include culturing a control group of COCs with no co-culture, as described further below. In some embodiments, a user may donate immature oocytes, such as germinal vesicle (GV) and Metaphase 1 (M1) oocytes that may be used in medium as part of the group culture to help grow COCs. Oocyte donation may follow an oocyte retrieval process as discussed above. A user participating in oocyte donation may be different, or the same, from the user related to the second biological sample containing immature COCs. In some embodiments, an oocyte donation user may undergo a stimulation protocol as disclosed above. In some embodiments, granulosa cells, cumulus cells, oocytes, GV oocytes, MI, oocytes, and all other types of cells described through this disclosure may be lysed, extracted for genomic material and flash frozen. For example, cells may undergo enzymatic cell lysis using enzymes such as lysozyme, lysostaphin, zymolase, cellulose, protease or glycanase, and the like. Other lysis methods may be applied such as, chemical lysis, detergent lysis, alkaline lysis, mechanical lysis, thermal lysis, acoustic lysis, physical lysis, non-mechanical lysis and the like. In some embodiments, culture media may be flash frozen. Freezing methods may include using a cryoprotective agent such as dimethyl sulfoxide and/or any other freezing method described through this disclosure.

Still referring to the apparatus of FIG. 1A, computing device 104 is configured to assign second biological sample 136 a scoring metric 152 as a function of culturing second biological sample 136.

vii. Culture Protocol

In apparatuses described herein (e.g., see FIG. 1B), computing device 104 is configured to assign the oocyte to a culture protocol 136 as a function of maturity level 128. In some embodiments, the assigned oocyte may be a denuded oocyte. A “culture protocol,” as used in this disclosure, is a cell culture process by which cells are grown under controlled conditions. Culture protocol 136 may include cell culture metabolites selected as a function of maturity level 128; and cell culture mediums selected as a function of maturity level 128. As used in this disclosure, a “cell culture metabolite” is a substance involved in cell metabolism that optimize the synthesis of new molecules in a cell culture. For example, a cell culture metabolite may include doxycycline. Computing device 104 may generate and train a culture classifier 140 using training data that may include correlations between the oocyte and or maturity level to cell culture protocols, for instance and without limitation in the form of training examples. Training examples may be derived from a metabolite index, cell medium index, protocol index, feedback index contained in a culture database. For example, culture classifier 140 may take determined GV oocytes from maturity classifier 132 and assign the oocyte a culture protocol 136 containing IVM media and 500 ng/mL of androstenedione. Culture classifier 140 training data may be received from a Culture database 144 and the biological sample. As used in this disclosure, Culture database 144″ is a database correlating scientific knowledge relating to cell culture processes. Culture database 144 may be communicatively connected to computing device 104 and implemented as described above. Culture database 144 may include a metabolite index, cell medium index, and a protocol index. As used in this disclosure, metabolite index” is a data structure relating to scientific knowledge concerning metabolites. The metabolite index may contain data regarding the effect of particular metabolites in a cell culture as it relates to maturity level 128 of the oocyte, along with dosing requirements and preparations. As used in this disclosure, a “cell medium index” is a data structure relating to scientific knowledge concerning cell culture mediums. For examples the cell medium index may contain data regarding optimal mediums used in cell cultivation and methods on preparation/storage. As used in this disclosure, a “protocol index” is a data structure correlating scientific knowledge regarding IVM cell culture procedures. For examples the protocol index may include co-culture and group culture methods used in IVM.

Referring to the apparatus of FIG. 1B, in some embodiments, culture protocol 136 may include culturing the oocyte with a co-culture containing granulosa cells produced from human induced pluripotent stem cells (hiPSCs). The cultured oocyte may be a denuded oocyte. As used in this disclosure, a “co-culture” is a cell cultivation set-up, in which two or more different populations of cells are grown with some degree of contact between them. hiPSCs may be produced using Clustered Regularly Interspaced Short Palindromic Repeats (CRISPR) technology. “CRISPR” is programmable technology that targets specific stretches of genetic code to edit DNA at precise locations. In some embodiments, CRISPR-based gene editing techniques can be used to introduce, into an iPSC genome, one or more genes encoding for factors that induce differentiation into ovarian support cells (e.g., ovarian granulosa cells). These factors include, e.g., FOXL2, NR5A1, GATA4, RUNX1, and RUNX2.

CRISPR technology may include CRISPR-CAS 9. Cas9 (or “CRISPR-associated protein 9”) is an enzyme that uses CRISPR sequences as a guide to recognize and cleave specific strands of DNA that are complementary to the CRISPR sequence. Cas9 enzymes together with CRISPR sequences form the basis of a technology known as CRISPR-Cas9 that can be used to edit genes within organisms. CRISPR technology may include Class 1 CRISPR systems including type I (cas3), type III (cas10), and type IV and 12 subtypes. CRISPR technology may include Class 2 CRISPR systems including type II (cas9), type V (cas12), type VI (cas13), and 9 subtypes. In some embodiments, CRISPR technology may involve CRISPR-Cas design tools which are computer software platforms and bioinformatics tools used to facilitate the design of guide RNAs (gRNAs) for use with the CRISPR/Cas gene editing system. For example, CRISPR-Cas design tools may include: CRISPRon, CRISPRoff, Invitrogen TrueDesign Genome Editor, Breaking-Cas, Cas-OFFinder, CASTING, CRISPy, CCTop, CHOPCHOP, CRISPOR, sgRNA Designer, Synthego Design Tool, and the like. CRISPR technology may also be used as a diagnostic tool. For example, CRISPR-based diagnostics may be coupled to enzymatic processes, such as SHERLOCK-based Profiling of in vitro Transcription (SPRINT). SPRINT can be used to detect a variety of substances, such as metabolites in subject samples or contaminants in environmental samples, with high throughput or with portable point-of-care devices.

Still referring to the apparatus of FIG. 1B, in some embodiments, a user may donate hiPSCs. hiPSCs donation may follow an oocyte retrieval process as discussed above. A user participating in hiPSCs donation may be different, or the same, from the user related to the biological sample. In some embodiments, hiPSCs donation user may undergo a stimulation protocol as disclosed above. In some embodiments regarding the assigned culture protocol 136, hiPSCs, granulosa cells, cumulus cells, oocytes, GV oocytes, MI, oocytes, and all other types of cells described through this disclosure may be lysed, extracted for genomic material and flash frozen as the final step in the culture process. For example, cells may undergo enzymatic cell lysis using enzymes such as lysozyme, lysostaphin, zymolase, cellulose, protease or glycanase, and the like. Other lysis methods may be applied such as, chemical lysis, detergent lysis, alkaline lysis, mechanical lysis, thermal lysis, acoustic lysis, physical lysis, non-mechanical lysis and the like. In some embodiments, culture media may be flash frozen. Freezing methods may include using a cryoprotective agent such as dimethyl sulfoxide and/or any other freezing method described throughout this disclosure.

Still referring to the apparatus of FIG. 1B, in embodiments regarding the culture of the oocyte, cell culture media may include LAG media. For example, LAG media may be used for the incubation of COCs post-retrieval from a stimulation protocol. Package size may be a 10 mL vial. Storage may be at 2-8° C. away from light up to one month. Media equilibration may be 18 to 24 hours pre-culture, include a seed 100 μl droplet and placed into 37° C. incubator with 6% O₂ and proper CO₂. In some embodiments, cell culture media may include IVM media. For example, a modified-MediCult IVM media may be used as a baseline control during the culturing process. Package size may be a 10 mL vial. Storage may be at 2-8° C. away from light up to one month. In some embodiments, the cell culture mediums may include metabolites. For example, the modified-MediCult IVM media may include human serum albumin, FSH, hCG, Androstenedione, Doxycycline and other compounds. Other cell culture material and equipment may include: liquid nitrogen, hyaluronidase, dPBS, IVF-Qualified mineral oil, universal GPS dishes, G-NOPS plus media, micropipettes, stripper pipettors, camera-equipped inverted ICSI Microscope, Dry Inject Tabletop incubators, saturated humidity incubators, EmbryoScope, microcentrifuge, refrigerator, −20° C. freezer to 100° C. freezer, liquid nitrogen storage dewer, 35 mm dishes for denuding, stripper pipette tips, and other components one skilled in the art would understand to be included in the cell culture process.

viii. Culture Data

In some apparatuses described herein (e.g., see FIG. 1A), computing device 104 is configured to receive culture data 140 relating to second biological sample 136. “Culture data” is data that provides a characterization of the biological, genetic, biochemical and/or physiological properties, compositions, or activities of cell cultured biological samples. Culture data 140 may include recording data and identifying growth trends of the COCs formed as a result of adding the specialized granulosa cells to the second biological extraction. In an embodiment, second biological sample 136 may rest in culture media for 2-3 hours after retrieval to allow for equilibration to in vitro conditions. Computing device 104 may receive culture data 140 from a culture sample database 144. A “culture sample database” is a database including analytical data regarding the culturing process and methods of second biological sample 136. For example, culture data 140 may be images of the cultured second biological sample 136, wherein computing device 104 may be configured to analyze the images for results, objectives, and the like. In some embodiments, computing device 104 may receive data such as embryologist notes regarding the process, results, objectives, and the like of cultured second biological sample 136 from culture sample database 144. Culture sample database 144 may be communicatively connected to computing device 104 and implemented as described above. In some embodiments, culture sample database 144 may include an oocyte analytical index. A “oocyte analytical index” is a data structure containing, rubrics, analytical methods, and approaches to analyzing cell culture media. For example, the oocyte analytical index may include methods to oocyte scoring, outcome analysis, confounding variable analysis, and the like.

In some apparatuses described herein (e.g., see FIG. 1B), computing device 104 is configured to receive culture data 148 as a function of culture protocol 136. “Culture data 148” is data that provides a characterization of the biological, genetic, biochemical and/or physiological properties, compositions, or activities of cell cultured biological samples. Culture data 148 may include recording data and identifying growth trends of the COCs formed as a result of adding the specialized granulosa cells, such as hiPSCs, to the denuded or non-denuded oocyte. Culture data 148 may be received from an oocyte index contained in Culture database 144. As used in this disclosure, a “culture sample index” is a database including analytical data regarding the culturing process and methods of the oocyte. For example, culture data 148 may be images of the cultured oocytes, wherein computing device 104 may be configured to analyze the images for results, objectives, and the like. In some embodiments, Culture database 144 may include an oocyte index. An “oocyte index” is a data structure containing, rubrics, analytical methods, and approaches to analyzing cell culture media. For example, the oocyte analytical index may include methods to oocyte scoring, outcome analysis, confounding variable analysis, and the like. In some embodiments, computing device 104 may receive data such as embryologist notes regarding the process, results, objectives, and the like of the cultured oocyte from a feedback index in Culture database 144. Computing device 104 may train a classifier or other machine-learning models configured to calculate a scoring metric using training data. In some embodiments, training data may include correlations between culture data to oocyte scoring, outcome analysis, confounding variable analysis for instance and without limitation in the form of training examples. Training examples may be derived from data in the oocyte index and feedback index retrieved from culture database 114.

ix. Scoring

A “scoring metric,” as used in this disclosure, is a measure of quantitative assessment used for comparing, and tracking performance or production of oocyte maturation. In an embodiment, a scoring metric may be calculated after denuding. “Denuding,” as used in this disclosure, is any process in which a cell may be removed from an oocyte. Denuding may include any mechanical and/or enzymatic process. For instance and without limitation denuding may include removing granulosa cells and/or cumulus cells from an oocyte. This may be performed mechanically and/or with one or more chemicals such as an enzyme to aid in the separation.

In some apparatuses described herein (e.g., see FIG. 1A) computing device 104 may receive subject information regarding the completion of the stimulation protocol 132 such as: subject Age, subject BMI, number of COCs retrieved, AMH Levels (μ/L), antral follicle count (AFC) at last ultrasound, subject oocyte retrieval day E2 Levels (ng/L), subject oocyte retrieval day P4 Levels (ng/L), subject oocyte retrieval day LH (IU/L), subject oocyte retrieval day FSH (IU/L), Days of stimulation, Gonadotropin used, and total injected dose (IU). Assigning the scoring metric 152 may include the computing device 104 analyzing imaged group culture of one or both of co-culture and no-co-culture growth groups. Computing device 104 may receive a: pre-culture group COC image, post-culture group COC image, and a post-culture denuded oocyte image. In some embodiments, images may be of frozen lysates and cell culture media. In an embodiment, scoring metric may include assessing a developmentally mature oocyte via microscopy for presence of a polar body. If a polar body is found, then the oocyte may be selected and utilized for intracytoplasmic sperm injection (ICSI) fertilization and/or oocyte freezing.

In some embodiments of the apparatuses described herein (e.g., see FIG. 1A and FIG. 1B), images may be sent to a third party for scoring assignment. A third party” is a qualified person or organization, such as an embryologist, to analyze the group cultures and develop/assign the scoring metric 152. Additionally, computing device 104 may perform any determinations, classification, and/or analysis steps, methods, processes, performed by a third party. In some embodiments the scoring metric 152 may include total oocyte scoring (TOS) as a function of analyzing the imaged group cultures. Oocyte scoring may include metrics such as shape, size, ooplasm characteristics, structure of the perivitelline space (PVS), zona pellucida (ZP), polar body (PB) morphology, and the like. “Oocyte scoring,” as used in this disclosure, is a grading system assessing the production and quality of matured human oocytes. For example, computing device 104 may be configured to perform the total oocyte scoring on both pre and post culture oocyte images for generation of the TOS metric on a scale system of −6 to +6. Computing device 104 may generate and/or train a machine-learning model including a classification algorithm (image classifier 148) to perform the total oocyte scoring. The training data may include any data described throughout this disclosure, such as subject information, follicular dynamics information, oocyte scoring metric 152, study sample sheet (such as oocyte scoring metric 152 instruction set). Image classifier 148 may take the group culture images as an input, and by utilizing the training data, output the total oocyte score. Training data may include from culture sample database 144 as described above.

A detailed disclosure of the machine learning model is described in further detail below. Regarding oocyte shape, if oocyte morphology is poor (dark general oocyte coloration and/or ovoid shape), it may be assigned a value of −1; if almost normal (less dark general oocyte coloration and less ovoid shape), it may be assigned a value of 0; if it is judged to be normal, it may be assigned a value of +1. Regarding oocyte size: if oocyte size is defined as abnormally small or large, it may be assigned a value −1 if size is below 120μ or greater 160μ. If the size is almost normal, i.e., did not deviate from normal by more than 10 μ, a value of 0 may be assigned, and a value of +1 may be assigned if oocyte size is within normal range >130μ and <150μ. Regarding ooplasm characteristics, if the ooplasm is very granular and/or very vacuolated and/or demonstrated several inclusions, a value of −1 may assigned. If it is only slightly granular and/or demonstrated only few inclusions, a value of 0 may be assigned. Absence of granularity and inclusions may result in a +1 value. Regarding structure of the perivitelline space (PVS), the PVS may defined as −1 with an abnormally large PVS, an absent PVS or a very granular PVS. It may be assigned a value of 0 with a moderately enlarged PVS and/or small PVS and/or a less granular PVS. A value of +1 may be assigned to a normal size PVS with no granules. Regarding, zona pellucida (ZP), if ZPs is very thin or thick (<10 μm or >20 μm) the oocyte may be assigned a −1. If the ZP does not deviate from normal by more than 2 μm it may be assigned 0. A normal zona (>12 μm and <18 μm) may be assigned a +1. Regarding polar body (PB) morphology, PB morphology is defined as follows: Flat and/or multiple PBs or zero PBs, granular and/or either abnormally small or large PBs is designated as −1. PBs, judged a fair but not excellent may be designated as 0, and a designation of +1 may be given to PBs of normal size and shape. In some embodiments, MII oocytes PB score may not be aggregated into TOS. In some embodiments, the TOS calculated by computing device 104 may be crossed checked against an embryologist or a similar person skilled in the art to solidify that the quality scoring was biased by image quality. Feedback relating to correction by a professional, adjustments, correlations may be added to the training data of the machine-learning model.

In some embodiments of the apparatuses described herein (e.g., see FIG. 1A and FIG. 1B), computing device 104 may train a classifier or other machine-learning models configured to calculate TOS using training data. In some embodiments, training data may include correlations between culture data to image quality and a 6-point qualitative scale for instance and without limitation in the form of training examples. Training examples may be derived from data in the oocyte scoring index and feedback index retrieved from culture database 144. A scoring metric 152 may include performing an outcome analysis as a function of the TOS. An “outcome analysis,” as used in the disclosure, can be: 1.) a measurement of the maturation rate and oocyte quality scores between cultures in the group culture; or 2.) a measurement of the maturation rate and oocyte quality scores between the control culture and co-culture. Parametric or non-parametric tests may be applied to determine the significance of findings during the analysis. Computing device 104 may use a classification algorithm using methods described above to determine GV to MII oocyte maturation rate; GV to MI oocyte maturation rate; MI to MII oocyte maturation rate; Average Total Oocyte Score; Average Oocyte Shape; Average Oocyte Size; Average Ooplasm quality; Average PVS quality; Average ZP quality; Average Polar Body quality, and the like. In some embodiments these outcomes may reported as a as mean, median, and deviation.

In some embodiments, and with continued reference to FIG. 1A or FIG. 1B, computing device 104 may conduct the outcome analysis using machine learning processes 116 and/or models as described throughout this disclosure.

In one example related to FIG. 1A, computing device 104 may train a machine-learning model to output an outcome analysis based on inputted group culture images, wherein the training data includes oocyte scoring metrics, study sample sheet, subject information, feedback from computing device 104 programmers/third parties, data from assigned stimulation protocol, and all other forms of data described through this disclosure. Training data may come from biological sample database 124 and culture database 144. In some embodiments, communications from a third party may be inputted into a machine learning process 116 to create a machine-learning model to generate the scoring metric. For example, a third-party communication may contain embryologist notes related to total oocyte scoring, wherein the notes are inputted into a machine-learning model containing a classifier to generate the outcome analysis using training data, received biological sample database 124 and culture database 144, containing subject information, data from image classifier 148, data from assigned stimulation protocol, study sample sheet, and any other form of data described throughout this disclosure. Additionally, or alternatively, communications relating to scoring metrics generated by the computing device may be sent to a third party may, using machine learning processes 116. For example, oocyte scoring metrics may be sent to a third party operated remote computing device communicatively connected to computing device 104, wherein the third party may conduct further analysis such as the outcome analysis. Furthering this example, a third-party response to communications generated by computing device 104 may be uploaded into a database communicatively connected to computing device 104 and be used as feedback in training data.

Still referring to FIG. 1A, in some embodiments, scoring metric 152 may include an Omics-based analysis. For example, frozen cell lysates and cell culture mediums may be analyzed for bulk RNA-sequencing, whole genome bisulfite sequencing (WGBS), mass spectrometry-based proteomics and metabolomics. Cell culture media may be utilized for metabolomics analysis to determine changes in molecular content of media following co-culture compared to pre-culture media controls. This may be utilized by computing device 104 to profile dynamic changes in paracrine signaling between granulosa cells and oocytes. The data gathered may then be aggregated for downstream analysis for determination of changes in epigenetic state, metabolite presence, and gene expression between different co-culture conditions and controls.

In another example related to FIG. 1B, computing device 104 may train a machine-learning model to output an outcome analysis based on inputted culture images, wherein the training data includes oocyte scoring metrics, study sample sheet, subject information, feedback from computing device 104 programmers/third parties, data from the stimulation protocol, and any other form of data described through this disclosure. Training data may come from biological sample database and culture database 144. In some embodiments, communications from a third party may be inputted into a machine learning process 116 to create a machine-learning model to generate scoring metric 152. For example, a third-party communication may contain embryologist notes related to total oocyte scoring, wherein the notes are inputted into a machine-learning model containing a classifier to generate the outcome analysis using training data, received from biological sample database and Culture database 144, containing subject information, data from image classifier, data from the stimulation protocol, study sample sheet, and any other form of data described throughout this disclosure. Additionally, or alternatively, communications relating to scoring metrics generated by the computing device may be sent to a third party may, using machine learning processes 116. For example, oocyte scoring metrics may be sent to a third party operated remote computing device communicatively connected to computing device 104, wherein the third party may conduct further analysis such as the outcome analysis. Furthering this example, a third-party response to communications generated by computing device 104 may be uploaded into a database communicatively connected to computing device 104 and be used as feedback in training data.

Still referring to FIG. 1B, in some embodiments, scoring metric may include an Omics-based analysis. Omics are novel, comprehensive approaches for analysis of complete genetic or molecular profiles of humans and other organisms. For example, in contrast to genetics, which focuses on single genes, genomics focuses on all genes (genomes) and their inter-relationships. In some embodiments, an omics-based analysis may include, genomics, proteomics, transcriptomics, pharmacogenomics, epigenomics, microbiomics, lipidomics, glycomics, transcriptomics culturomics, and/or any other omics one skilled in the art would understand as applicable. In some embodiments, after cultivation, an oocyte that has failed to mature, showing GV or MI characteristics, may be harvested for single cell RNA-sequencing, along with their associated granulosa cells from their culture. For this, oocytes and granulosa cells may be flash frozen and for library preparation. Of the oocytes that display MII oocyte development, half may be harvested for single cell RNA-sequencing along with their associated granulosa cells using the above flash freeze methods described throughout this disclosure. The remaining half of MII oocytes may be utilized for proteomic studies. The culture media for all conditions may additionally be flash frozen and utilized for metabolomics and proteomics to identify cholesterol metabolite levels and paracrine protein production. For example, frozen cell lysates and cell culture mediums may be analyzed for bulk RNA-sequencing, whole genome bisulfite sequencing (WGBS), mass spectrometry-based proteomics and metabolomics. Cell culture media may be utilized for metabolomics analysis to determine changes in molecular content of media following co-culture compared to pre-culture media controls. This may be utilized by computing device 104 to profile dynamic changes in paracrine signaling between granulosa cells and oocytes. As the media components are flash frozen, the sample is effectively quenched and amenable to metabolic assessment. The data gathered may then be aggregated for downstream analysis for determination of changes in epigenetic state, metabolite presence, and gene expression between different co-culture conditions and controls.

### B. Machine-Learning Module

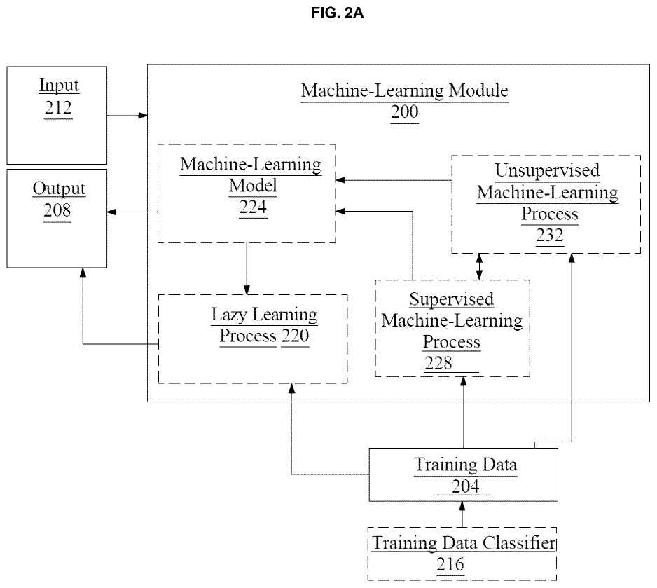

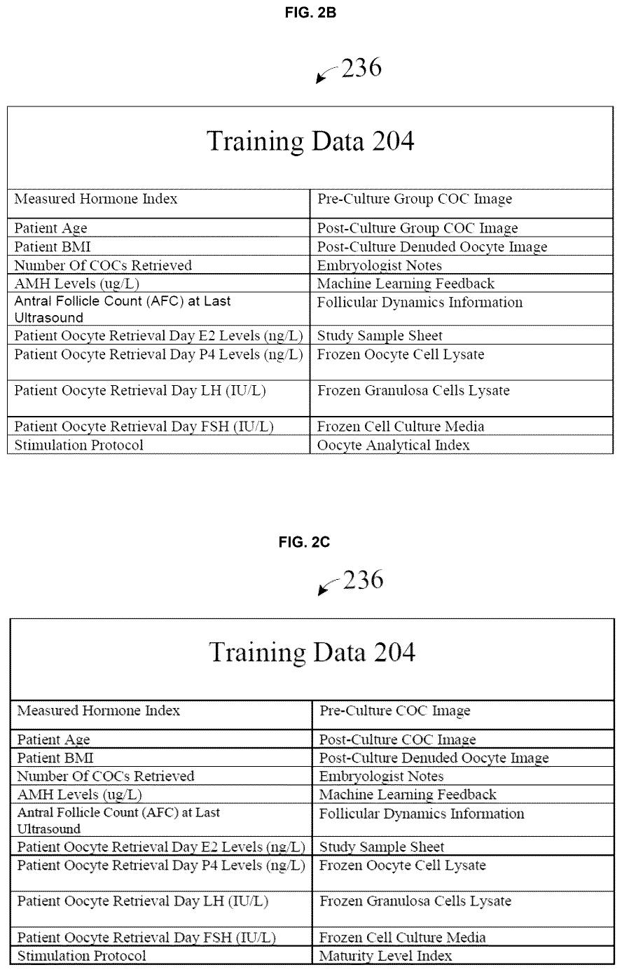

Referring now to the machine learning module of FIG. 2A, an exemplary embodiment of a machine-learning module 200 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 204 to generate an algorithm that will be performed by a computing device/module to produce outputs 208 given data provided as inputs 212; this is in contrast to a non-machine learning software program where the commands to be executed are determined in advance by a user and written in a programming language.

i. Training Data

Still referring to the machine learning module of FIG. 2A, “training data,” as used herein, is data containing correlations that a machine-learning process may use to model relationships between two or more categories of data elements. For instance, and without limitation, training data 204 may include a plurality of data entries, each entry representing a set of data elements that were recorded, received, and/or generated together; data elements may be correlated by shared existence in a given data entry, by proximity in a given data entry, or the like. Multiple data entries in training data 204 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 204 according to various correlations; correlations may indicate causative and/or predictive links between categories of data elements, which may be modeled as relationships such as mathematical relationships by machine-learning processes as described in further detail below. Training data 204 may be formatted and/or organized by categories of data elements, for instance by associating data elements with one or more descriptors corresponding to categories of data elements. As a non-limiting example, training data 204 may include data entered in standardized forms by persons or processes, such that entry of a given data element in a given field in a form may be mapped to one or more descriptors of categories. Elements in training data 204 may be linked to descriptors of categories by tags, tokens, or other data elements; for instance, and without limitation, training data 204 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively, or additionally, and continuing to refer to FIG. 2A, training data 204 may include one or more elements that are not categorized; that is, training data 204 may not be formatted or contain descriptors for some elements of data. Machine-learning algorithms and/or other processes may sort training data 204 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 204 to be made applicable for two or more distinct machine-learning algorithms as described in further detail below. Training data 204 used by machine-learning module 200 may correlate any input data as described in this disclosure to any output data as described in this disclosure.

ii. Training Data Classifier

Further referring to FIG. 2A, training data may be filtered, sorted, and/or selected using one or more supervised and/or unsupervised machine-learning processes and/or models as described in further detail below; such models may include without limitation a training data classifier 216. Training data classifier 216 may include a “classifier,” which as used in this disclosure is a machine-learning model as defined below, such as a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. Machine-learning module 200 may generate a classifier using a classification algorithm, defined as a process whereby a computing device and/or any module and/or component operating thereon derives a classifier from training data 204. Classification may be performed using, without limitation, linear classifiers such as without limitation logistic regression and/or naïve Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers.

iii. Lazy Learning Process

Still referring to FIG. 2A, machine-learning module 200 may be configured to perform a lazy-learning process 220 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 204. Heuristic may include selecting some number of highest-ranking associations and/or training data 204 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

iv. Machine Learning Model

Alternatively, or additionally, and with continued reference to FIG. 2A, machine-learning processes as described in this disclosure may be used to generate machine-learning models 224. A “machine-learning model,” as used in this disclosure, is a mathematical and/or algorithmic representation of a relationship between inputs and outputs, as generated using any machine-learning process including without limitation any process as described above and stored in memory; an input is submitted to a machine-learning model 224 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 224 may be generated by creating an artificial neural network, such as a convolutional neural network comprising an input layer of nodes, one or more intermediate layers, and an output layer of nodes. Connections between nodes may be created via the process” of “training” the network, in which elements from a training data 204 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 2A, machine-learning algorithms may include at least a supervised machine-learning process 228. At least a supervised machine-learning process 228, as defined herein, include algorithms that receive a training set relating a number of inputs to a number of outputs, and seek to find one or more mathematical relations relating inputs to outputs, where each of the one or more mathematical relations is optimal according to some criterion specified to the algorithm using some scoring function. For instance, a supervised learning algorithm may include as described above as inputs, as described above outputs, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 204. Persons skilled in the art, upon reviewing the entirety of this disclosure, will be aware of various possible variations of at least a supervised machine-learning process 228 that may be used to determine relation between inputs and outputs. Supervised machine-learning processes may include classification algorithms as defined above.

Further referring to FIG. 2A, machine learning processes may include at least an unsupervised machine-learning processes 232. An unsupervised machine-learning process, as used herein, is a process that derives inferences in datasets without regard to labels; as a result, an unsupervised machine-learning process may be free to discover any structure, relationship, and/or correlation provided in the data. Unsupervised processes may not require a response variable; unsupervised processes may be used to find interesting patterns and/or inferences between variables, to determine a degree of correlation between two or more variables, or the like.

Still referring to FIG. 2A, machine-learning module 200 may be designed and configured to create a machine-learning model 224 using techniques for development of linear regression models. Linear regression models may include ordinary least squares regression, which aims to minimize the square of the difference between predicted outcomes and actual outcomes according to an appropriate norm for measuring such a difference (e.g., a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g.,. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 2A, machine-learning algorithms may include, without limitation, linear discriminant analysis. Machine-learning algorithm may include quadratic discriminate analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include various forms of latent space regularization such as variational regularization. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized tress, AdaBoost, gradient tree boosting, and/or voting classifier methods. Machine-learning algorithms may include neural net algorithms, including convolutional neural net processes.

### C. Training Data

Referring now to FIG. 2B, shown is an exemplary table 236 of training data 204. Training data 204 may include any data described throughout this disclosure. For example, training data 204 may contain de-identified user information. A user may be referred to as a subject in this disclosure. De-identified subject information may include subject Age, subject BMI, number of COCs retrieved, AMH Levels (μ/L), antral follicle count (AFC) at last ultrasound, subject oocyte retrieval day E2 Levels (ng/L), subject oocyte retrieval day P4 Levels (ng/L), subject oocyte retrieval day LH (IU/L), subject oocyte retrieval day FSH (IU/L), days of stimulation, gonadotropin used, total injected dose (IU), and the like. Additionally or alternatively, training data 204 may include pre-culture group COC images, post-culture group COC images, post-culture denuded oocyte images, third party notes such as embryologist notes, machine-learning feedback, follicular dynamics information, study sample sheet, frozen oocyte cell lysate data, frozen granulosa cells lysate data, frozen cell culture media data, data from systemic hormone index, data from oocyte analytical index, data from biological sample database 124, data from culture database 144, and the like.

Referring now to FIG. 2C, shown is an exemplary table 236 of training data 204. Training data 204 may include any data described throughout this disclosure. For example, training data 204 may contain de-identified user information. A user may be referred to as a subject in this disclosure. De-identified subject information may include subject Age, subject BMI, number of COCs retrieved, AMH Levels (μ/L), antral follicle count (AFC) at last ultrasound, subject oocyte retrieval day E2 Levels (ng/L), subject oocyte retrieval day P4 Levels (ng/L), subject oocyte retrieval day LH (IU/L), subject oocyte retrieval day FSH (IU/L), days of stimulation, gonadotropin used, total injected dose (IU), and the like.

Additionally or alternatively, training data 204 may include pre-co-culture COC images, post-co-culture COC images, post-culture denuded oocyte images, third party notes such as embryologist notes, machine-learning feedback, follicular dynamics information, study sample sheet, frozen oocyte cell lysate data, frozen granulosa cells lysate data, frozen cell culture media data, data from systemic hormone index, data from maturity index, data from biological sample database 124, data from culture database 144, and the like.

### D. Mini Stimulation Protocol

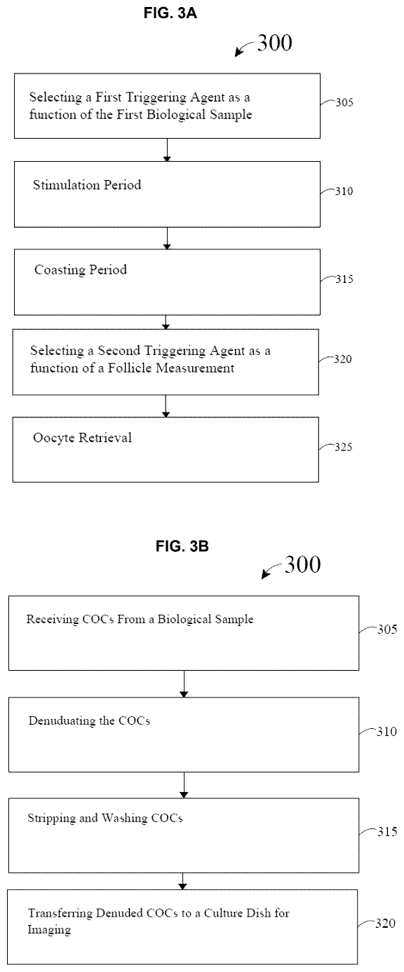

Referring now to FIG. 3A, shown is an exemplary flow chart of a mini stimulation protocol 300. At step 305, minimal stimulation protocol may include selecting a first triggering agent (e.g., a follicular triggering agent) as a function of the first biological sample to inject a user with. The selected first triggering agent may be selected based on the measured hormone levels of the user. The first triggering agent may include a human recombinant follicle stimulating hormone (rFSH). A rFSH triggering agent may include for example, Gonal-F as produced by Merck Global, Follistim as produced by Merck Global; Follitropin Alfa as produced by Teva, headquartered in Tel Aviv-Yafo, Israel; and Glucophage as produced by Merck Global. rFSH, or any other triggering agent (e.g., a follicular triggering agent) as described throughout this disclosure, may be injected into the user at different increments a plurality of times. In some embodiments, a triggering agent may not be administered to a subject. In an embodiment, timing as to when minimal stimulation may be initiated by a subject may be determined by a subject's contraception status as described above in more detail. For example, a subject who is not taking contraception may begin stimulation with rFSH on the second day of the subject's menstrual cycle. In yet another non-limiting example, a subject who is taking contraception such as a combined oral contraception (COC) pill may begin stimulation 5 days after the last pill was consumed. Drug dosage and selection may be determined by one or more lab tests such as a blood test taken on the second day of a subject's menstrual cycle to determine blood levels of E2, FSH, LH, p4, and/or AMH. One or more measurements may be utilized to determine ovarian reserve health, circulating hormone levels, and/or fertility status.

At step 310, protocol 300 may include stimulating the user over the span of a time period such as 3 days with the first triggering agent. For example, rFSH may be injected in an amount of 100-2001U three or more times over the span of a 1-4 day stimulation period. For example, the stimulation period may span over 3 days. After injection of the first triggering agent (e.g., a follicular triggering agent) an ultrasound may be performed to determine an average follicle size of the cell, such as an oocyte cell. At step 315, protocol may include a day coasting period. A coasting period includes any coasting period as described above as described in more detail. A coasting period may include where a second triggering agent is withheld until serum estradiol (E2) has decreased to what is considered to be, by one skilled in the art, a safe level to prevent the onset of ovarian hyperstimulation syndrome. In some embodiments, an ultrasound may be performed after the 3-day miniature stimulation protocol 300 during a coasting period in order to determine the average follicle size of the cell. In some embodiments, the coasting period may span over 2 days. Determining the average follicle size of the cell may include identifying when the average follicle size is between 8-12 nm. At step 320, as a function of determining the average follicle size of the cell, a second triggering agent may be injected into the user. The second triggering agent may include a human chorionic gonadotropin (hCG). The second triggering agent may be dosed based on one or more factors pertaining to the user including follicle size, previous diagnosis of any medical condition, ultrasound imaging, drug allergy, subject tolerance of a particular medication and the like. A rFSH triggering agent may include for example, Pregnyl as produced by Schering Plough, headquartered in Kenilworth, NJ; Novarel as produced by Ferring Laboratories, headquartered in Parsippany, NJ; Chorex as produced by Encocam, headquartered in Huntingdon, England; and Profasi as produced by Serum Institute of India Ltd, headquartered in Pune, India. In some embodiments, the second triggering may be any triggering agent as described throughout this disclosure. Similar to the first triggering agent, the second triggering agent may be injected into the user at different increments a plurality of times. For example, in an amount ranging from 200 μg-700 μg, injected once or a plurality of times over the span of the 3-day stimulation period. At step 325, after the injection of the second triggering agent, a cell may be retrieved for the user, wherein the cell includes an oocyte cell and/or a COC. For example, after the coasting period, at 8-9 mm follicle size, a 500 μg hCG trigger agent may be administered, with oocyte retrieval at 36 hours post-administration. Oocyte retrieval may include a medical professional, such as a doctor inserting the extraction device into the follicle containing an egg and extracting the egg and surrounding fluid. Oocyte retrieval may include retrieval of immature oocytes, mature oocytes, COCs, and any other type of cell involved in reproduction found in the ovaries. Oocyte retrieval may occur during a time frame from anywhere ranging from 12-96 hours after hCG administration. In an embodiment, a blood test to examine levels of hormones such as E2, LH, and/or P4 may be measured to ensure for one or more quality metrics and to check that a subject took the hCG as prescribed. This may also aid in determining if hormone levels are within standard predicted value ranges.

### E. Oocyte Denudation

Referring now to FIG. 3B, shown is an exemplary flow chart of oocyte denudation. At step 305, method 300 may include receiving COCs from a biological sample. COCs may be received following oocyte retrieval methods disclosed above. At step 310, method 300 may include oocyte denudation of the COCs. In some embodiments, denudation may occur in a IVM well, by gently mechanically disassociating cells by pipetting to remove most cumulus and/or granulosa cells. If enzymatic disassociation is needed, the cells may be transferred to a separate dish for hyaluronidase treatment. At step 315, COCs may be stripped with stripper tips and washed in IVM media or MOPS plus media to clean the oocyte for imaging and if needed inactivate hyaluronidase. Stripper tips may include 200 micron and or 400 microns for fine cleaning. In some embodiments, GVs and MI oocytes may be formulated and utilized in cultivation as a result of the denudation of the COCs. At step 320, method 300 may include transferring denuded COCs to a culture dish for imaging.

### F. Co-Culture

Referring now to FIG. 4, shown is an exemplary table 400 of metabolite formulations that may be included in cultures described herein. Metabolite 404 column lists exemplary metabolites that may be used as a triggering agent and/or cell culture metabolite. A “cell culture metabolite,” as used in this disclosure, is a substance involved in cell metabolism that optimize the synthesis of new molecules in a cell culture. Stock Solution Preparation Concentration 408 column lists exemplary concentrations for cell culture metabolites. Final Concentration in IVM Media 412 column list exemplary concentrations of cell culture metabolites in a IVM media for group culturing or coculturing of a first or a second biological sample 136. For example, 10 mg/mL of HSA may be added to an IVM media for any cell culturing described herein (e.g., group culturing or coculturing). About 75 mUI/mL of FSH may be added to an IVM media for any cell culturing described herein. About 100 mUI/mL of hCG may be added to an IVM media for any cell culturing described herein. About 500 ng/mL of androstenedione may be added to an IVM media for any cell culturing described herein (e.g., group culturing or coculturing). About 1 μg/mL of doxycycline may be added to an IVM media for any cell culturing described herein. In some embodiments, steroidogenic granulosa cells, derived from human induced pluripotent stem cells (hiPSCs), may be co-cultured with denuded or non-denuded immature oocytes (e.g., COCs), thereby reconstituting the follicular niche in vitro to promote rapid and efficient oocyte maturation in a manner that reinforces oocyte health and developmental competence. As used in this disclosure, a “steroidogenic granulosa cell” is a granulosa cell expressing high levels of steroidogenic enzymes, such as estradiol. For example, a steroidogenic granulosa cell may be a mural granulosa cell extracted from the antral follicle. Applying steroidogenic granulosa cells in the co-cultures of oocytes (e.g., COCs) may increase oocyte maturation in vitro after egg/oocyte retrieval, allowing for utilization of all retrieved eggs/oocyte by directly supplying nutrients, raw materials, and mechanical support to oocytes throughout gametogenesis and folliculogenesis. Steroidogenic granulosa cells may grow and perform oocyte maturation of immature oocytes in standard IVF and IVM media. This may increase the overall pool of available, healthy oocytes for use in IVF and reduce the number of egg/oocyte retrieval procedures a user is subjected to.

i. Preparing a Granulosa Co-Culture

Referring now to FIG. 5, depicted is an exemplary flow-chart 500 for preparing a granulosa co-culture is illustrated. Granulosa cells in a subject's ovaries play a key role in the female reproductive system. These cells release estrogen, progesterone and other hormones which drive oocyte maturation in the ovary, making them a logical tool for application in IVM. Furthermore, granulosa cells provide the developmental niche for follicle and oocyte development, directly supplying nutrients, raw materials, and mechanical support to oocytes throughout gametogenesis and folliculogenesis. At step 505, a liquid nitrogen frozen granulosa cell cryovial may be thawed and incubated. In some embodiments thawing and incubation may occur up to 72 hours before oocyte retrial from a user. The granulosa cell cryovial may be a 1 mL cryovial containing 50,000 to 500,000 granulosa cells. In some embodiments, the granulosa cell cryovial may be a 1 mL cryovial containing 100,000 granulosa cells. Thawing may occur by placing the granulosa cell cryovial in a water bath or a dry bead bath. The granulosa cell cryovial may be incubated for 3 to 5 minutes. At step 510, IVM media may be added to the cryovial for cell suspension. In some embodiments, 0.5 ml of ICM media may be added to induce cell suspension. At step 515, the cell suspension is transferred to a tube and centrifuged. In some embodiments, 1 mL of cell suspension may be transferred to a 1.5 mL tube, wherein the tube is centrifuged at 300×g for 5 minutes. At step 520, a pipette may be used to remove a supernatant. Pipette may be a p1000 pipette. At step 525, cells pellet formed as a function of centrifuged tube containing the cell suspension, may be resuspended in a IVM media and centrifuged. IVM media may be a 1 mL IVM media. The tube may be centrifuged as described above. At step 530, a pipette may be used to remove a supernatant. Pipette may be a p1000 pipette. At step 535 cells pellet may be resuspend in a IVM media. IVM media may be a 0.1 mL IVM media. At this step, granulosa cell may now be at 1,000 cells per 1 ul. In some embodiments 10ul of the cell suspension may be utilized per oocyte in second biological sample 136 related to a user.

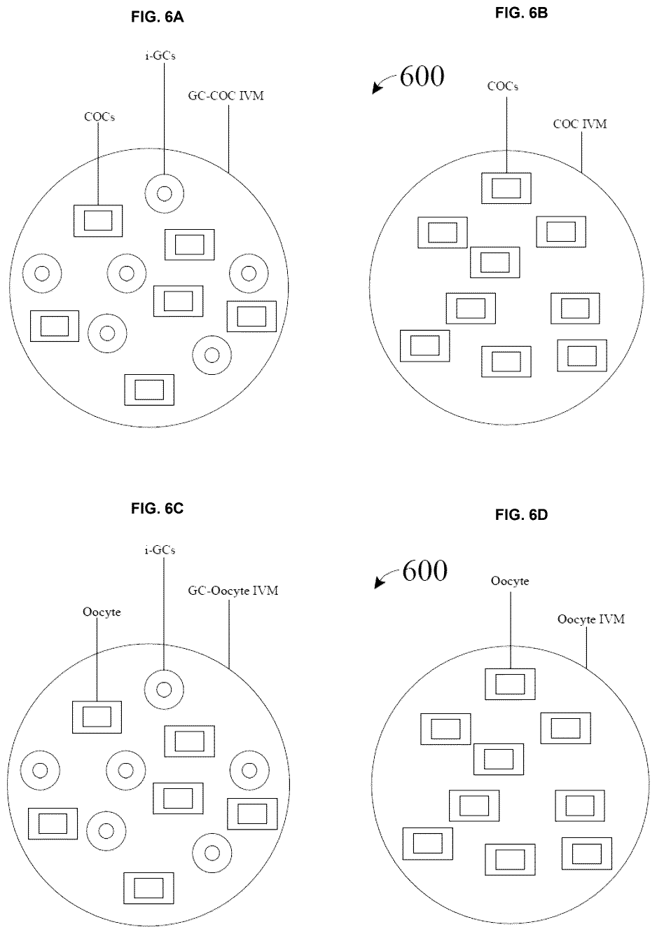

Referring now to FIG. 6A, depicted is an exemplary embodiment of a co-cultured second biological sample 136 including immature COCs related to the user. In some embodiments, COCs received after oocyte retrieval from a follicular aspirate relating to the user may be randomly divided in half to into a media, such as a Lag media of a granulosa cell plate, and the other half may go into a LAG media of a no-co-culture plate. COCs may be incubated in the LAG media at 37 C for 2 hours. Granulosa cells may be prepared as described in FIG. 5. The prepared granulosa cells may be added to the right center well that contain IVM media, adding 10,000 granulosa cells per COC that may be cultured. The dish with the granulosa cell may then be placed back in the incubator until use. After the 2-hour incubation period, the COCs in the LAG media may be transferred to the IVM media in the granulosa cell dish with a Pasteur pipette.

Referring now to FIG. 6B, depicted is an exemplary embodiment of a control group culture of second biological sample 136 including immature COCs related to the user. COCs may be incubated in a LAG media at 37° C. for 2 hours. After the 2-hour incubation period, the COCs in the LAG media may be transferred to IVM media in a control dish with a Pasteur pipette.

Referring now to FIG. 6C, depicted is an exemplary embodiment of a co-cultured oocyte including immature oocytes related to the user. In some embodiments, oocytes may be denuded oocytes. Oocytes received after oocyte retrieval from a follicular aspirate relating to the user may be randomly divided in half to into a media, such as a LAG media of a granulosa cell plate, and the other half may go into a LAG media of a no-co-culture plate. Alternatively, some oocytes for culture may be pre-frozen, in which case they may first be thawed before culture placement. Oocytes may be incubated in the LAG media at 37° C. for 2 hours. Granulosa cells may be prepared as described in FIG. 5. The prepared granulosa cells may be added to the surrounding 50 μl wells that contain IVM media, adding 10,000 granulosa cells per oocyte that may be cultured. The dish with the granulosa cell may then be placed back in the incubator until use. After the 2-hour incubation period, the oocyte in the LAG media may be transferred to the IVM media in the granulosa cell dish with a Pasteur pipette. In the granulosa co-culture, after 18 to 48 hours incubation, the culture plate may be removed, and the oocytes and granulosa cells may be imaged in their individual wells. In some embodiments the culture may be imaged after 24 hours.

Referring now to FIG. 6D, depicted is an exemplary embodiment of a control culture of immature oocytes related to the user. Oocytes may be incubated in a LAG media at 37° C. for 2 hours. After the 2-hour incubation period, the oocyte in the LAG media may be transferred to an IVM media in a control dish with a Pasteur pipette. In the control culture, after 10 to 48 hours incubation, the culture plate may be removed, and the oocytes may be imaged in their individual wells. In some embodiments the culture may be imaged after 24 hours.

ii. Inducing Human Oocyte Maturation In Vitro

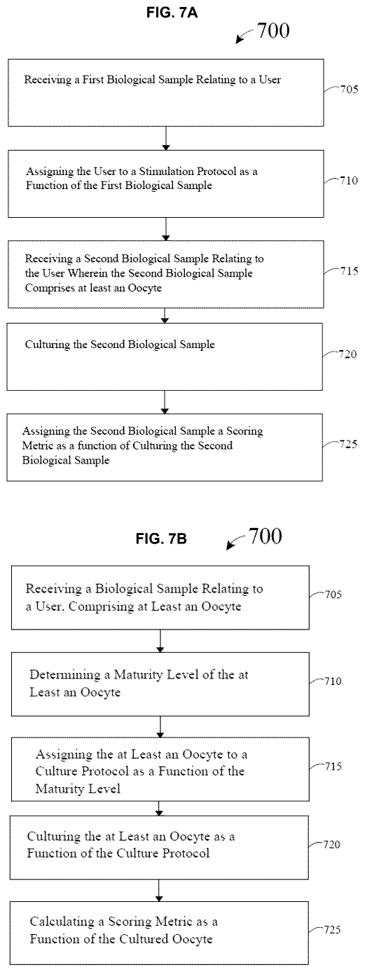

Referring now to FIG. 7A, depicted is a flow diagram of an exemplary method for inducing human oocyte maturation in vitro. Method 700 may include using computing device 104 (e.g., of FIG. 1A) to carry out steps to be listed. At step 705, method 700 includes receiving a first biological sample relating to a user. The first biological sample may be any form of biological sample as defined and exemplified throughout this this disclosure. For example, the first biological sample may include a blood sample from a user as exemplified, at least, in FIG. 1. A user may be a user as defined in FIG. 1A, such as a person. In some embodiments, the biological sample may be extracted from the user through an extraction device, as defined and exemplified, at least, in FIG. 1A. For example, the extraction device may include a medical syringe to draw blood from the user. Biological samples may also include systemic hormones. At step 710, method 700 includes assigning the user to a stimulation protocol as a function of the first biological sample. A stimulation protocol is a medication injection process as defined and exemplified, at least, in FIG. 1A. In some embodiments, the stimulation protocol may be assigned based on a measured hormone level of the biological sample. The measured hormone level, as defined in FIG. 1A, may include E2, LH, FSH, and/or P4 levels. The assigned stimulation protocol may include a minimal stimulation protocol configured to trigger the release of a cell in the span of 3 days as defined and exemplified, at least, in FIG. 1. In an embodiment, the minimal stimulation protocol may include selecting a first triggering agent as a function of the first biological sample and selecting a second triggering agent as a function of a follicle measurement. A triggering agent, as defined in FIG. 1A, may include human Serum Albumin, FSH, hCG, androstenedione, and doxycycline in formulation described in FIGS. 1-6 in this disclosure.

Still referring to step 710, in some embodiments, the minimal stimulation protocol may include injecting a user with a first triggering agent; performing an ultrasound to determine an average follicle size of the cell; injecting the user with a second triggering agent; and retrieving the cell, wherein the cell includes an oocyte cell from the user. The first triggering agent may include a human recombinant follicle stimulating hormone (rFSH). rFSH may be injected into the user at different increments a plurality of times. For example, and with reference to FIG. 1A, rFSH may be injected in an amount of 100-2001U three or more times over the span of a 3-day stimulation period. After injection of the first triggering agent an ultrasound may be performed to determine an average follicle size of the cell, such as an oocyte cell. In some embodiments, the ultrasound may be performed, after the 3-day stimulation protocol, during a 2-day coasting period, as defined in FIG. 1A. Determining the average follicle of the cell may include identifying when the average follicle size is between 8-12 nm. As a function of determining the average follicle size of the cell, a second triggering agent may be injected into the user. The second triggering agent may include a human chorionic gonadotropin (hCG). Similar to the first triggering agent, the second triggering agent may be injected into the user at different increments a plurality of times. For example, and with reference to FIG. 1A, the second triggering agent may in an amount ranging from 200 μg-700 μg, injected once or a plurality of times over the span of the 3-day stimulation period. After the injection of the second triggering agent, a cell may be retrieved for the user, wherein the cell includes an oocyte cell. For example, and with reference to FIG. 1A, after the coasting period, at 8-9 mm follicle size, a 500 μg hCG trigger may be administered, with oocyte retrieval at 36 hours post-administration. Oocyte retrieval may include a medical professional, such as a doctor inserting the extraction device into the follicle containing an egg and extracting the and surrounding fluid.

Still referring to FIG. 7A, method 700 at step 715, includes receiving a second biological sample relating to the user wherein the second biological sample includes at least an immature Cumulus-Oocyte complex (COCs) as defined in FIG. 1A. The second biological sample may include bodily fluids as described above. The second biological sample may be extracted using an extraction device and received as disclosed above. At step 720, method 700 includes culturing the second biological sample. In some embodiments, culturing the second biological sample may include culturing the Cumulus-Oocyte complexes in a group culture, as defined in FIG. 1A. For example, and with reference to FIGS. 1-6, group culturing may include culturing the Cumulus-Oocyte complexes with a granulosa co-culture and a control group of COCs with no co-culture. In some embodiments cell culture media may include LAG media. For example, and with reference to FIGS. 1-6, LAG media may be used for the incubation of COCs post-retrieval from the stimulation protocol. Package size may be a 10 mL vial. Storage may be at 2-8° C. away from light up to one month. Media equilibration may be 18 to 24 hours pre-culture, include a seed 100 μl droplet and placed into 37° C. incubator with 6% O₂ and proper CO₂. In some embodiments, cell culture media may include IVM media. For example, and with reference to FIGS. 1-6, a modified-MediCult IVM media may be used as a baseline control during the culturing process. Package size may be a 10 mL vial. Storage may be at 2-8° C. away from light up to one month. In some embodiments, the cell culture mediums may include metabolites. For example, the modified-MediCult IVM media may include human serum albumin, FSH, hCG, androstenedione, doxycycline and other compounds. Other cell culture material and equipment may include: liquid nitrogen, hyaluronidase, dPBS, IVF-Qualified mineral oil, universal GPS dishes, G-NOPS plus media, micropipettes, stripper pipettors, camera-equipped inverted ICSI Microscope, Dry Inject Tabletop incubators, saturated humidity incubators, EmbryoScope, microcentrifuge, 4° C. refrigerator, −20° C. freezer, −80° C. freezer, liquid nitrogen storage dewer, 35 mm dishes for denuding, stripper pipette tips, and other components one skilled in the art would understand to be included in the cell culture process.

Still referring to FIG. 7A, at step 725, method 700 includes assigning the second biological sample a scoring metric as a function of culturing the second biological sample. Assignment may be based subject information regarding the completion of the stimulation protocol such as: subject Age, subject BMI, number of COCs retrieved, AMH Levels (μ/L), antral follicle count (AFC) at last ultrasound, subject oocyte retrieval day E2 Levels (ng/L), subject oocyte retrieval day P4 Levels (ng/L), subject oocyte retrieval day LH (IU/L), subject oocyte retrieval day FSH (IU/L), Days of stimulation, Gonadotropin used, and total injected dose (IU). In some embodiments, assignment of the scoring metric may include imaging the group cultures and analyzing the images of one or both of co-culture and no-co-culture growth groups. For example, group culture images may contain a pre-culture group COC image, a post-culture group COC image, and a post-culture denuded oocyte image. In some embodiments, images may be sent to a third party, as defined in FIG. 1A, for scoring assignment. In some embodiments the scoring metric 152 may include total oocyte scoring (TOS) as a function of analyzing the imaged group cultures. Oocyte scoring may include metrics such as shape, size, ooplasm characteristics, structure of the perivitelline space (PVS), zona pellucida (ZP), polar body (PB) morphology, and the like as described in detail at least in FIG. 1A. Total oocyte scoring on both pre and post culture oocyte images for generation of the TOS metric may be based on a scale system of −6 to +6.

In some embodiments, the scoring metric may include performing an outcome analysis as a function of the TOS as defined and exemplified in FIG. 1A. Parametric or non-parametric tests may be applied to determine the significance of findings during the analysis. Outcome analysis may be used to determine GV to MII oocyte maturation rate; GV to MI oocyte maturation rate; MI to MII oocyte maturation rate; Average Total Oocyte Score; Average Oocyte Shape; Average Oocyte Size; Average Ooplasm quality; Average PVS quality; Average ZP quality; Average Polar Body quality, and the like. In some embodiments these outcomes may reported as a as mean, median, and deviation. Still referring to step 725, in some embodiments, the scoring metric may include an Omics-based analysis. For example, and with reference to FIG. 1A, frozen cell lysates and cell culture mediums may be analyzed for bulk RNA-sequencing, whole genome bisulfite sequencing (WGBS), mass spectrometry-based proteomics and metabolomics.

In any apparatus described herein (e.g., see FIG. 1A and FIG. 1B) cell culture media may be utilized for metabolomics analysis to determine changes in molecular content of media following co-culture compared to pre-culture media controls. This may be utilized to profile dynamic changes in paracrine signaling between granulosa cells and oocytes. The data gathered may then be aggregated for downstream analysis for determination of changes in epigenetic state, metabolite presence, and gene expression between different co-culture conditions and controls.

iii. In Vitro Oocyte Rescue Post Stimulation

Referring now to FIG. 7B, depicted is an exemplary flow diagram illustrating a method for oocyte rescue in vitro post stimulation, for example, and with reference to FIG. 1-6. Method 700 may include using computing device 104 (e.g., of FIG. 1B) or any computing device described throughout this disclosure (e.g., see FIG. 1A and FIG. 1B). In some embodiments, method 700 may include using a third party as defined and described in FIG. 1B. At step 705, method includes receiving a biological sample relating to a user, including at least an oocyte, for example, with reference to FIGS. 1-6. In some embodiments, an oocyte may be an immature oocyte. In some embodiments, an immature oocyte may be a plurality of oocytes. An immature oocyte may be immature cumulus-oocyte-complexes (COCs) taken from the mother. In some embodiments, the immature oocyte may contain an oocyte wherein the specialized granulosa cell is added to mature the oocyte in a cell culture and thus create a COC. In some embodiments, the biological sample may include bodily fluids including blood, saliva, urine, semen (seminal fluid), vaginal secretions, cerebrospinal fluid (CSF), synovial fluid, pleural fluid (pleural lavage), pericardial fluid, peritoneal fluid, amniotic fluid, nasal fluid, optic fluid, gastric fluid, breast milk, cell culture supernatants, and the like. In some embodiments, and with reference to FIG. 1B, a biological sample containing an oocyte may be retrieved from a user post simulation by a medical professional, such as a doctor inserting an extraction device into the follicle containing an egg and extracting the egg and surrounding fluid. The extraction device may include a needle, syringe, vial, lancet, Evacuated Collection Tubes (ECT), tourniquet, vacuum extraction tube systems, any combination thereof and the like. For example, the extraction device may comprise a butterfly needle set. Oocyte retrieval may include retrieval of immature oocytes, mature oocytes, COCs, and any other type of cell involved in reproduction found in the ovaries.

Still referring to FIG. 7B, at step 710, method 700 includes determining a maturity level of the at least an oocyte, for example, and with reference to FIG. 1B and FIG. 3B. In some embodiments, the maturity level may be an assessment of the oocyte maturation stage of oogenesis. Determining the maturity level of the oocyte may include denuding the oocyte, for example and with reference to FIG. 1B. Oocyte denudation may include enzymatic and mechanical methods with the help of hyaluronidase and sterile glass pipettes as described in FIG. 1B and FIG. 3B. At step 715, method 700 includes assigning the oocyte to a culture protocol as a function of the maturity level. The culture protocol may include cell culture metabolites selected as a function of the maturity level; and cell culture mediums selected as a function of maturity level, for example and with reference to FIG. 1B. In some embodiments, the culture protocol may include culturing the oocyte with a with a granulosa co-culture containing granulosa cells sourced from human induced pluripotent stem cells (hiPSCs). The cultured oocyte may be a denuded oocyte. hiPSCs may be produced using Clustered Regularly Interspaced Short Palindromic Repeats (CRISPR) technology as described above. In some embodiments, a user may donate hiPSCs. hiPSCs donation may follow an oocyte retrieval process as discussed above. A user participating in hiPSCs donation may be different, or the same, from the user related to the biological sample. In some embodiments, hiPSCs donation user may undergo a stimulation protocol as disclosed above. In some embodiments regarding the assigned culture protocol, hiPSCs, granulosa cells, cumulus cells, oocytes, GV oocytes, MI, oocytes, and all other types of cells described throughout this disclosure may be lysed, extracted for genomic material and flash frozen as the final step in the culture process, for example and with reference to FIGS. 1-6.

Still referring to FIG. 7B, at step 720, method 700 includes culturing the at least an oocyte as a function of the culture protocol, for example and with reference to FIGS. 1-6. In embodiments regarding the culture of the oocyte, cell culture media may include LAG media. In some embodiments, cell culture media may include IVM media. In some embodiments, the cell culture mediums may include metabolites. At step 725, method 700 includes calculating a scoring metric as a function of the cultured oocyte, for example and with reference to FIG. 1B. Calculating the scoring metric may include analyzing imaged co-cultures and control cultures. Images may contain a: pre-culture oocyte image, post-culture oocyte image, and a post-culture denuded oocyte image. In some embodiments, images may be of frozen lysates and cell culture media. In some embodiments the scoring metric may include total oocyte scoring (TOS) as a function of analyzing the imaged cultures. For example, each oocyte image may be subjected to a total oocyte scoring (TOS) system, which measures oocyte health via a 6-point qualitative scale as described above. In some embodiments, the scoring metric may include oocyte scoring. Oocyte scoring may include metrics such as shape, size, ooplasm characteristics, structure of the perivitelline space (PVS), zona pellucida (ZP), polar body (PB) morphology, and the like. In some embodiments, scoring metric may include performing an outcome analysis as a function of the TOS. An “outcome analysis,” as used in the disclosure, is a measurement of the maturation rate and oocyte quality scores between the control culture and co-culture. Parametric or non-parametric tests may be applied to determine the significance of findings during the analysis. The outcome analysis may determine GV to MII oocyte maturation rate; GV to MI oocyte maturation rate; MI to MII oocyte maturation rate; Average Total Oocyte Score; Average Oocyte Shape; Average Oocyte Size; Average Ooplasm quality; Average PVS quality; Average ZP quality; Average Polar Body quality, and the like. In some embodiments these outcomes may reported as a as mean, median, and deviation. In some embodiments, scoring metric may include an Omics-based analysis. In some embodiments, after cultivation, an oocyte that has failed to mature, showing GV or MI characteristics, may be harvested for single cell RNA-sequencing, along with their associated granulosa cells from their culture. For this, oocytes and granulosa cells may be flash frozen and for library preparation. Of the oocytes that display MII oocyte development, half may be harvested for single cell RNA-sequencing along with their associated granulosa cells using the above flash freeze methods described throughout this disclosure. The remaining half of MII oocytes may be utilized for proteomic studies. The culture media for all conditions may additionally be flash frozen and utilized for metabolomics and proteomics to identify cholesterol metabolite levels and paracrine protein production. Cell culture media may be utilized for metabolomics analysis to determine changes in molecular content of media following co-culture compared to pre-culture media controls. This may be utilized to profile dynamic changes in paracrine signaling between granulosa cells and oocytes. The data gathered may then be aggregated for downstream analysis for determination of changes in epigenetic state, metabolite presence, and gene expression between different co-culture conditions and controls.

### G. Software

It is to be noted that any one or more of the aspects and embodiments described herein may be conveniently implemented using one or more machines (e.g., one or more computing devices that are utilized as a user computing device for an electronic document, one or more server devices, such as a document server, and the like) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

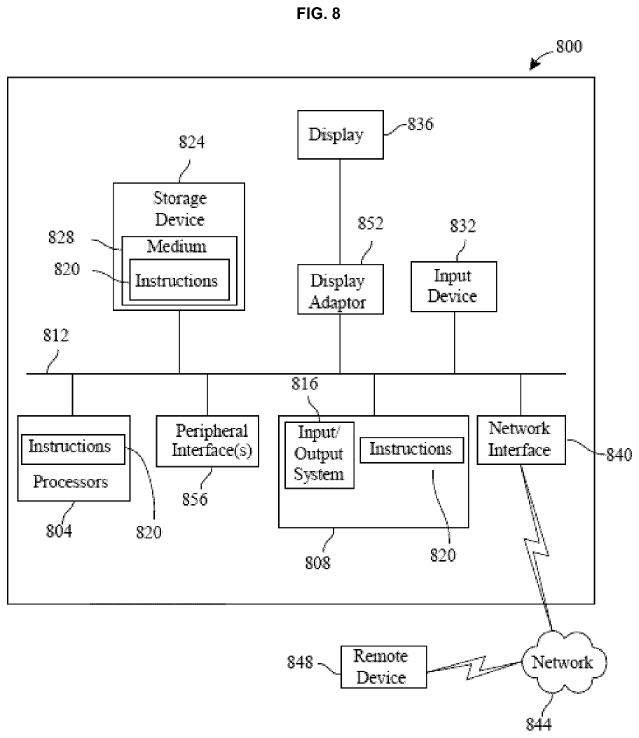

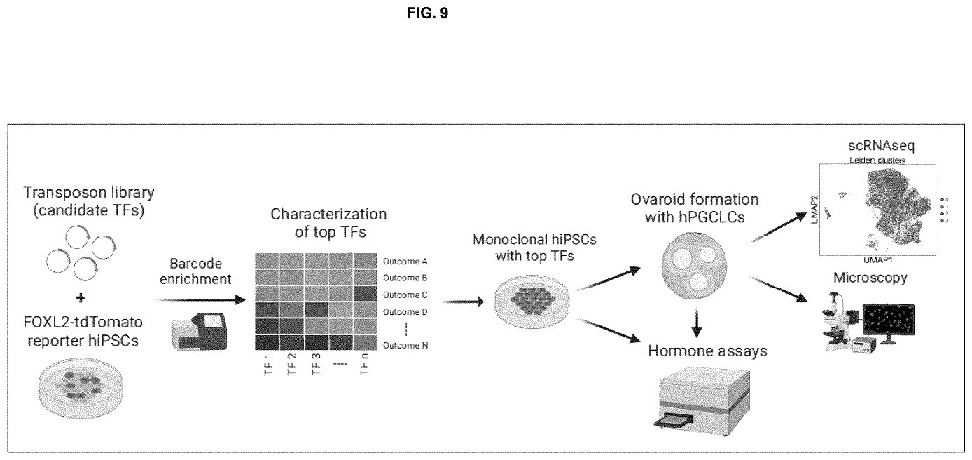

FIG. 8 shows a diagrammatic representation of one embodiment of a computing device in the exemplary form of a computer system 800 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 800 includes a processor 804 and a memory 808 that communicate with each other, and with other components, via a bus 812. Bus 812 may include any of several types of bus structures including, but not limited to, a memory bus, a memory controller, a peripheral bus, a local bus, and any combinations thereof, using any of a variety of bus architectures.

Processor 804 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 804 may be organized according to Von Neumann and/or Harvard architecture as a non-limiting example. Processor 804 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), and/or system on a chip (SoC).

Memory 808 may include various components (e.g., machine-readable media) including, but not limited to, a random-access memory component, a read only component, and any combinations thereof. In one example, a basic input/output system 816 (BIOS), including basic routines that help to transfer information between elements within computer system 800, such as during start-up, may be stored in memory 808. Memory 808 may also include (e.g., stored on one or more machine-readable media) instructions (e.g., software) 820 embodying any one or more of the aspects and/or methodologies of the present disclosure. In another example, memory 808 may further include any number of program modules including, but not limited to, an operating system, one or more application programs, other program modules, program data, and any combinations thereof.

Computer system 800 may also include a storage device 824. Examples of a storage device (e.g., storage device 824) include, but are not limited to, a hard disk drive, a magnetic disk drive, an optical disc drive in combination with an optical medium, a solid-state memory device, and any combinations thereof. Storage device 824 may be connected to bus 812 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 824 (or one or more components thereof) may be removably interfaced with computer system 800 (e.g., via an external port connector (not shown)). Particularly, storage device 824 and an associated machine-readable medium 828 may provide nonvolatile and/or volatile storage of machine-readable instructions, data structures, program modules, and/or other data for computer system 800. In one example, software 820 may reside, completely or partially, within machine-readable medium 828. In another example, software 820 may reside, completely or partially, within processor 804.

Computer system 800 may also include an input device 832. In one example, a user of computer system 800 may enter commands and/or other information into computer system 800 via input device 832. Examples of an input device 832 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 832 may be interfaced to bus 812 via any of a variety of interfaces (not shown) including, but not limited to, a serial interface, a parallel interface, a game port, a USB interface, a FIREWIRE interface, a direct interface to bus 812, and any combinations thereof. Input device 832 may include a touch screen interface that may be a part of or separate from display 836, discussed further below. Input device 832 may be utilized as a user selection device for selecting one or more graphical representations in a graphical interface as described above.

A user may also input commands and/or other information to computer system 800 via storage device 824 (e.g., a removable disk drive, a flash drive, etc.) and/or network interface device 840. A network interface device, such as network interface device 840, may be utilized for connecting computer system 800 to one or more of a variety of networks, such as network 844, and one or more remote devices 848 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 844, may employ a wired and/or a wireless mode of communication. In general, any network topology may be used. Information (e.g., data, software 820, etc.) may be communicated to and/or from computer system 800 via network interface device 840.

Computer system 800 may further include a video display adapter 852 for communicating a displayable image to a display device, such as display device 836. Examples of a display device include, but are not limited to, a liquid crystal display (LCD), a cathode ray tube (CRT), a plasma display, a light emitting diode (LED) display, and any combinations thereof. Display adapter 852 and display device 836 may be utilized in combination with processor 804 to provide graphical representations of aspects of the present disclosure. In addition to a display device, computer system 800 may include one or more other peripheral output devices including, but not limited to, an audio speaker, a printer, and any combinations thereof. Such peripheral output devices may be connected to bus 812 via a peripheral interface 856. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, apparatuses, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

### EXAMPLES

### Example 1. a Method of Follicle Stimulation for Ovarian Release of Oocytes

This example demonstrates how a subject undergoing an ART procedure can be minimally stimulated with a triggering agent that reduces a hormonal burden on the subject.

A 35-year old female subject with polycystic ovarian syndrome (PCOS) undergoing ART procedures is examined by a clinician on day 2 of her menstrual cycle. An ultrasound analysis by the clinician determines that the subject's ovaries produce less than or equal to 20 oocytes (e.g., 1 to 5 oocytes, 4 to 10 oocytes, 8 to 16 oocytes, or 15 to 20 oocytes, e.g., 1 oocyte, 2 oocytes, 3 oocytes, 4 oocytes, 5 oocytes, 6 oocytes, 7 oocytes, 8 oocytes, 9 oocytes, 10 oocytes, 11 oocytes, 12 oocytes, 13 oocytes, 14 oocytes, 15 oocytes, 16 oocytes, 17 oocytes, 18 oocytes, 19 oocytes, 20 oocytes); thus, she is determined to have a reduced ovarian reserve.

The subject is administered a triggering agent (e.g., 100-2001U of human recombinant follicle stimulating hormone (rFSH)) to stimulate follicular maturation and oocyte release. Administration of the triggering agent begins on day 2±1 day (e.g., day 1, day 2, or day 3) of her menstrual cycle and continues daily for 1 to 4 days (e.g., 1 day, 2 days, 3 days, or 4 days). The subject's follicle size is monitored by an ultrasound until the average follicle size reaches about 8-10 mm (e.g., 7.5 mm, 8 mm, 8.5 mm, 9 mm, 9.5 mm, 10 mm, 10.5 mm, or more), upon which the oocytes (or a group of cells containing an oocyte, e.g., cumulus oocyte complex (COCs)) are retrieved from the subject by an aspiration-based methodology. For example, oocyte retrieval may utilize a transvaginal ultrasound with a needle guide on the probe to suction release follicular contents. Oocyte-containing follicular contents (e.g., follicular aspirates) are after washed with HEPES media (G-MOPS Plus, Vitrolife®), filtered with a 70-micron cell strainer (Falcon®, Corning), and examined on a dissection microscope. Oocytes (or a group of cells containing an oocyte, e.g., COCs) are transferred to culture dishes and media to begin co-culturing with granulosa cells.

### Example 2. a Method of Follicle Stimulation for Ovarian Release of Oocytes and In Vitro Maturation of Oocytes

This example demonstrates minimal follicle stimulation of a subject with a low ovarian reserve followed by oocyte harvest and in vitro maturation.

i. Follicle Stimulation for Ovarian Release of Oocytes

A 30-year old female subject receives a blood test that detects an anti-Mullerian hormone (AMH) level of less than or equal to 6 ng/mL (e.g., 1 ng/mL, 2 ng/mL, 3 ng/mL, 4 ng/mL, 5 ng/mL, or 6 ng/mL). Thus, she is determined to have a reduced ovarian reserve. Additional blood tests revealing that her estradiol level is between 20 and 50 μg/mL (e.g., 20-30 μg/mL, 25-35 μg/mL, 30-40 μg/mL, 35-45 μg/mL, or 40-50 μg/mL; e.g., 20 μg/mL, 21 μg/mL, 22 μg/mL, 23 μg/mL, 24 μg/mL, 25 μg/mL, 30 μg/mL, 35 μg/mL, 40 μg/mL, 45 μg/mL, or 50 μg/mL) reaffirms the determination of the reduced ovarian reserve.

The subject is administered a triggering agent (e.g., 50 mg of clomiphene citrate) to stimulate follicular maturation and oocyte release. Since the subject is taking a hormonal contraceptive, administration of the triggering agent begins on or about day 5±1 day (e.g., day 4, day 5, or day 6) after taking her last contraceptive and continues daily for 1 to 4 days (e.g., 1 day, 2 days, 3 days, or 4 days). The subject's follicle size is monitored by an ultrasound until the average follicle size reaches about 8-10 mm (e.g., 7.5 mm, 8 mm, 8.5 mm, 9 mm, 9.5 mm, 10 mm, 10.5 mm, or more), upon which the oocytes (or a group of cells containing an oocyte, e.g., COCs) are retrieved from the subject by an aspiration-based methodology. For example, oocyte retrieval may utilize a transvaginal ultrasound with a needle guide on the probe to suction release follicular contents. Oocyte-containing follicular contents (e.g., follicular aspirates) are after washed with HEPES media (G-MOPS Plus, Vitrolife®), filtered with a 70-micron cell strainer (Falcon®, Corning), and examined on a dissection microscope. Oocytes (or a group of cells containing an oocyte, e.g., COCs) are transferred to culture dishes containing cell culture media (e.g., IVM, IVF, or LAG media) for about 1 to 3 hours (e.g., 1 hour, 2 hours, or 3 hours) before introducing granulosa cells for co-culture.

ii. In Vitro Maturation of Oocytes

If present, cultured COCs may be separated from their cumulus cells (and any other non-oocyte cells) in a process referred herein as oocyte denudation. Oocyte denudation is performed on COCs in an IVM well by mechanically disassociating cells by pipetting to remove the cumulus and/or granulosa cells. Additional oocyte denudation may be performed with an enzymatic disassociation (e.g., hyaluronidase treatment). COCs may be stripped with stripper tips and washed in IVM media or MOPS plus media to clean the oocyte for imaging and, if needed, to inactivate hyaluronidase. Stripper tips include 200 micron and/or 400 microns for fine cleaning.

Next, germinal vesical stage (GVs) and metaphase I stage (MI) oocytes are co-cultured with about 50,000-100,000 (e.g., 50,000-60,000 cells, 60,000-70,000 cells, 70,000-80,000 cells, 80,000-90,000 cells, or 90,000-100,000 cells; e.g., 50,000 cells, 55,000 cells, 60,000 cells, 65,000 cells, 70,000 cells, 75,000 cells, 80,000 cells, 85,000 cells, 90,000 cells, 95,000 cells, or 100,000 cells) granulosa cells (e.g., specialized granulosa cells, hiPSC-derived granulosa cells, or steroidogenic granulosa cells, as described herein). Metaphase II stage (MII) oocytes (e.g., oocytes with a polar body in the perivitelline space) can be properly frozen for storage. Co-culturing of oocytes and granulosa cells is for about 12-120 hours (e.g., 12-24 hours, 12-36 hours, 24-48 hours, 36-60 hours, 54-72 hours, 68-96 hours, 96-120 hours; e.g., 12 hours, 14 hours, 16 hours, 18 hours, 20 hours, 22 hours, 24 hours, 26 hours, 28 hours, 30 hours, 32 hours, 34 hours, 36 hours, 38 hours, 40 hours, 42 hours, 44 hours, 46 hours, 48 hours, 50 hours, 52 hours, 54 hours, 56 hours, 58 hours, 60 hours, 62 hours, 64 hours, 66 hours, 68 hours, 70 hours, 72 hours, 74 hours, 76 hours, 78 hours, 80 hours, 82 hours, 84 hours, 86 hours, 88 hours, 90 hours, 92 hours, 94 hours, 96 hours, 98 hours, 100 hours, 102 hours, 104 hours, 106 hours, 108 hours, 110 hours, 112 hours, 114 hours, 116 hours, 118 hours, or 120 hours).

Following co-culture, any one or more oocytes are utilized for assisted reproduction technology (ART) procedures. For example, oocytes may be utilized for intracytoplasmic sperm injection (ICSI).

### Example 3. Administration of a Follicular Triggering Agent

This example demonstrates the administration of a triggering agent to a subject.

A 30-year old female subject receives a blood test that detects estradiol levels between 20 and 50 μg/mL (e.g., 20-30 μg/mL, 25-35 μg/mL, 30-40 μg/mL, 35-45 μg/mL, or 40-50 μg/mL; e.g., 20 μg/mL, 21 μg/mL, 22 μg/mL, 23 μg/mL, 24 μg/mL, 25 μg/mL, 30 μg/mL, 35 μg/mL, 40 μg/mL, 45 μg/mL, or 50 μg/mL). The subject is administered multiple injections of a triggering agent over 1 to 4 days (e.g., 1 day, 2 days, 3 days, or 4 days) but no more than 5 days. The subject may receive multiple injections over multiple days such that a subject receives five dose injections of one or multiple triggering agents. For example, a subject receives three days of stimulation using 300 IU to 700 IU of rFSH per injection (e.g., 300-500 IU, 400-600 IU, 500-700 IU, 300-350 IU, 350-400 IU, 400-450 IU, 450-500 IU, 500-550 IU, 550-600 IU, 600-650 IU, 650-700 IU; e.g., 300 IU, 325 IU, 350 IU, 375 IU, 400 IU, 425 IU, 450 IU, 475 IU, 500 IU, 525 IU, 550 IU, 575 IU, 600 IU, 625 IU, 650 IU, 675 IU, or 700 IU) with one or more injections per day. In another example, the subject receives injections of hCG as a triggering agent using 200-700 μg or 2,500-10,000 IU hCG (e.g., 200-500 μg, 300-600 μg, 400-700 μg, 200-300 μg, 300-400 μg, 400-500 μg, 500-600 μg, or 600-700 μg). In yet another example, the subject receives one or more (e.g., 1, 2, 3, 4, or 5) doses of clomiphene citrate at 50-150 mg (e.g., 50-75 mg, 60-80 mg, 75-100 mg, 90-115 mg, 110-130 mg, 125-150 mg; e.g., 50 mg, 60 mg, 70 mg, 80 mg, 90 mg, 100 mg, 110 mg, 120 mg, 130 mg, 140 mg, 150 mg) per dose.

### Example 4. An Apparatus for Performing Follicle Stimulation and In Vitro Maturation of Oocytes

In this example, reference is made to FIG. 8 which shows a diagrammatic representation of one embodiment of a computing device in the exemplary form of a computer system 800 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 800 includes a processor 804 and a memory 808 that communicate with each other, and with other components, via a bus 812. Bus 812 may include any of several types of bus structures including, but not limited to, a memory bus, a memory controller, a peripheral bus, a local bus, and any combinations thereof, using any of a variety of bus architectures.

Processor 804 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 804 may be organized according to Von Neumann and/or Harvard architecture as a non-limiting example. Processor 804 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), and/or system on a chip (SoC).

Memory 808 may include various components (e.g., machine-readable media) including, but not limited to, a random-access memory component, a read only component, and any combinations thereof. In one example, a basic input/output system 816 (BIOS), including basic routines that help to transfer information between elements within computer system 800, such as during start-up, may be stored in memory 808. Memory 808 may also include (e.g., stored on one or more machine-readable media) instructions (e.g., software) 820 embodying any one or more of the aspects and/or methodologies of the present disclosure. In another example, memory 808 may further include any number of program modules including, but not limited to, an operating system, one or more application programs, other program modules, program data, and any combinations thereof.

Computer system 800 may also include a storage device 824. Examples of a storage device (e.g., storage device 824) include, but are not limited to, a hard disk drive, a magnetic disk drive, an optical disc drive in combination with an optical medium, a solid-state memory device, and any combinations thereof. Storage device 824 may be connected to bus 812 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 824 (or one or more components thereof) may be removably interfaced with computer system 800 (e.g., via an external port connector (not shown)). Particularly, storage device 824 and an associated machine-readable medium 828 may provide nonvolatile and/or volatile storage of machine-readable instructions, data structures, program modules, and/or other data for computer system 800. In one example, software 820 may reside, completely or partially, within machine-readable medium 828. In another example, software 820 may reside, completely or partially, within processor 804.

Computer system 800 may also include an input device 832. In one example, a user of computer system 800 may enter commands and/or other information into computer system 800 via input device 832. Examples of an input device 832 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 832 may be interfaced to bus 812 via any of a variety of interfaces (not shown) including, but not limited to, a serial interface, a parallel interface, a game port, a USB interface, a FIREWIRE interface, a direct interface to bus 812, and any combinations thereof. Input device 832 may include a touch screen interface that may be a part of or separate from display 836, discussed further below. Input device 832 may be utilized as a user selection device for selecting one or more graphical representations in a graphical interface as described above.

A user may also input commands and/or other information to computer system 800 via storage device 824 (e.g., a removable disk drive, a flash drive, etc.) and/or network interface device 840. A network interface device, such as network interface device 840, may be utilized for connecting computer system 800 to one or more of a variety of networks, such as network 844, and one or more remote devices 848 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 844, may employ a wired and/or a wireless mode of communication. In general, any network topology may be used. Information (e.g., data, software 820, etc.) may be communicated to and/or from computer system 800 via network interface device 840.

Computer system 800 may further include a video display adapter 852 for communicating a displayable image to a display device, such as display device 836. Examples of a display device include, but are not limited to, a liquid crystal display (LCD), a cathode ray tube (CRT), a plasma display, a light emitting diode (LED) display, and any combinations thereof. Display adapter 852 and display device 836 may be utilized in combination with processor 804 to provide graphical representations of aspects of the present disclosure. In addition to a display device, computer system 800 may include one or more other peripheral output devices including, but not limited to, an audio speaker, a printer, and any combinations thereof. Such peripheral output devices may be connected to bus 812 via a peripheral interface 856. Examples of a peripheral interface include, but are not limited to, a serial port, a USB connection, a FIREWIRE connection, a parallel connection, and any combinations thereof.

### Example 5: Materials and Methods for Examples 6 Through 8

We have developed human ovarian support cells (OSCs) generated from human induced pluripotent stem cells (hiPSCs) that hold the ability to recapitulate dynamic ovarian function in vitro. Here we investigate the potential of these OSCs to improve human oocyte maturation, retrieved from abbreviated gonadotropin stimulated cycles, as a co-culture system applied to IVM. We reveal that OSC-IVM significantly improves maturation rates compared to available IVM systems. Most importantly, we demonstrate OSC-assisted IVM oocytes are capable of robust euploid blastocyst formation, a key marker of their clinical utility. Together, these findings demonstrate a novel approach to IVM with broad applicability to modern IVF practice.

Specifically, to determine if in vitro maturation (IVM) of human oocytes can be improved by co-culture with ovarian support cells (OSCs) derived from human induced pluripotent stem cells (hiPSCs), oocyte donors were recruited to undergo abbreviated gonadotropin stimulation with or without hCG triggers and cumulus oocyte complexes (COCs) were allocated between the OSC-IVM condition and media only IVM controls.

Oocyte donors between the ages of 19 to 37 years were recruited for donation under informed consent, with an anti-mullerian hormone (AMH) value of greater than 1 ng/mL as inclusion criteria. The OSC-IVM culture condition was composed of 100,000 OSCs in suspension culture with human chorionic gonadotropin (hCG), recombinant follicle stimulating hormone (rFSH), androstenedione and doxycycline supplementation. IVM controls lacked OSCs and contained the same supplementation or only FSH and hCG.

Primary endpoints consisted of metaphase II (MII) formation rate and morphological quality assessment. A limited cohort of oocytes were additionally utilized for fertilization and blastocyst formation with PGT-A analysis. OSC-IVM resulted in a statistically significant improvement in MII formation rate compared to the media only control. OSC-IVM resulted in a statistically significant improvement in MII formation rate compared to a commercially available IVM control. Oocyte morphological quality between OSC-IVM and controls did not significantly differ. OSC-IVM improved maturation, fertilization, cleavage, blastocyst formation, high quality blastocyst formation and euploid blastocyst formation compared to the commercially available IVM control.

As a conclusion, the novel OSC-IVM platform is an effective tool for maturation of human oocytes obtained from abbreviated gonadotropin stimulation cycles, yielding improved blastocyst formation. OSC-IVM shows broad utility for different stimulation regimens, including hCG triggered truncated IVF and untriggered traditional IVM cycles making it a highly useful tool for modern fertility treatment.

i. Collection of Cumulus Oocyte Complexes (COCs)

### Subject Ages, IRB and Informed Consent

Subjects were enrolled in the study through Ruber Clinic (Madrid, Spain), Spring Fertility Clinic (New York, USA) and Pranor Clinic (Lima, Peru) using informed consent (CNRHA 47/428973.9/22, IRB #20225832, Western IRB, and Protocol #GC-MSP-01 respectively). Subject ages ranged between 19 and 37 years of age. Oocytes retrieved from the Ruber and Pranor clinics were utilized for maturation analysis endpoints only, while oocytes retrieved from Spring Fertility were utilized for embryo formation endpoints.

### Stimulation Characteristics

Twenty-five subjects received 3-4 days of stimulation using 300-600 IU of rFSH with an hCG trigger in preparation for immature oocyte aspiration for Experiment 1, with an AMH value of >1 ng/mL (see below). Twenty-one subjects received three consecutive days of 200 IU of rFSH with an hCG trigger in preparation for immature oocyte aspiration for Experiment 2, with an AMH value of >1.5 ng/mL used as an inclusion criterion to enrich for donors yielding more oocytes (see below). Six subjects received three to five doses of clomiphene citrate (100 mg) with an additional one to two doses of 150 IU rFSH with or without an hCG trigger for Experiment 2 with the goal of subsequent embryo formation, and an AMH value of >2.0 ng/mL was utilized as an inclusion criterion (see below). Gonadotropin injections were initiated on day 2 of a natural cycle or on the fifth day following cessation of oral contraceptive pills. A complete table of donor stimulation regimens for each donor in the study is provided in Table 1 below.

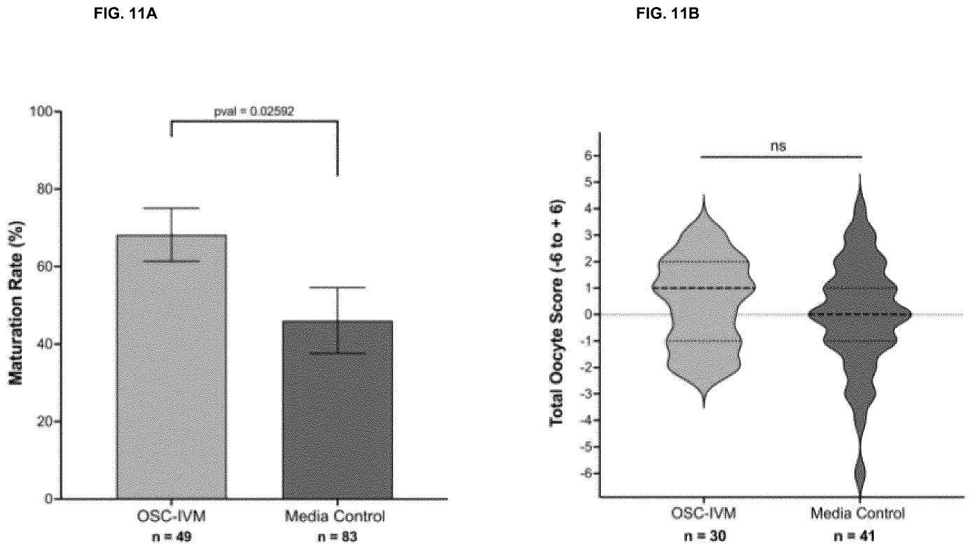

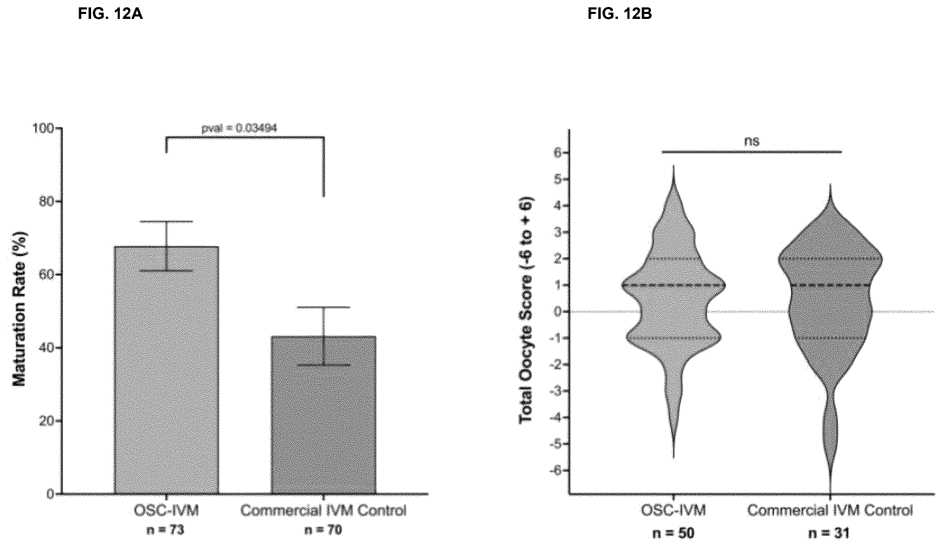

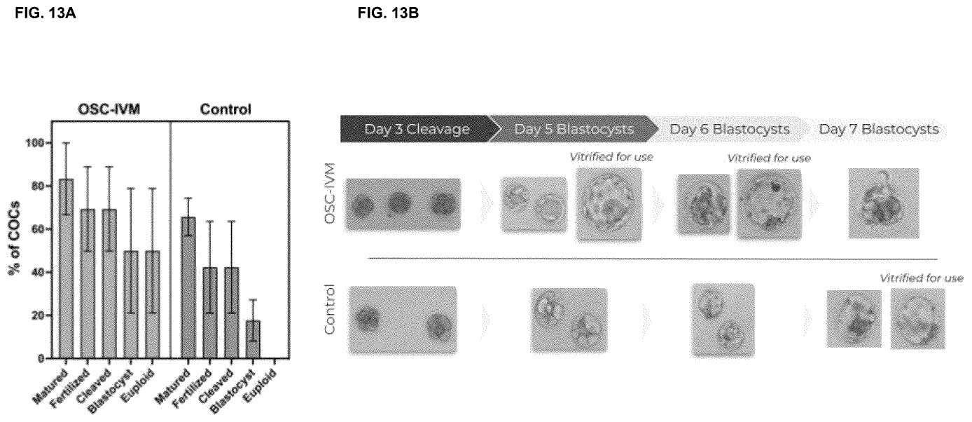

TABLE 1
Donor stimulation regimens

| Oocyte | | | | | | | | | |
|---|---|---|---|---|---|---|---|---|---|
| Donor | Study | Donor | | Clomid | rFSH | FSH | hCG | Oocytes | Text |
| Clinic | ID | Age | Donor BMI | (mg) | (IU) | Regimen | (IU) | Retrieved | Reference |
| Pranor | P01 | 25 | 55.00 | 0 | 450 | 150-150- | 10,000 | 7 | FIG. 11, |
| | | | | | | 150 | | | Table 3 |
| Pranor | P02 | 25 | 45.00 | 0 | 450 | 150-150- | 10,000 | 6 | FIG. 11, |
| | | | | | | 150 | | | Table 3 |
| Pranor | P03 | 19 | 50.00 | 0 | 450 | 150-150- | 10,000 | 3 | FIG. 11, |
| | | | | | | 150 | | | Table 3 |
| Pranor | P04 | 24 | 58.00 | 0 | 375 | 125-125- | 10,000 | 3 | FIG. 11, |
| | | | | | | 125 | | | Table 3 |
| Pranor | P05 | 21 | 57.00 | 0 | 325 | 125-125- | 10,000 | 9 | FIG. 11, |
| | | | | | | 75 | | | Table 3 |
| Pranor | P06 | 23 | 53.00 | 0 | 325 | 125-125- | 10,000 | 11 | FIG. 11, |
| | | | | | | 75 | | | Table 3 |
| Ruber | R01 | 25 | 24.00 | 0 | 600 | 200-200- | 10,000 | 3 | FIG. 11, |
| | | | | | | 200 | | | Table 3 |
| Ruber | R02 | 20 | 22.00 | 0 | 600 | 200-200- | 10,000 | 10 | FIG. 11, |
| | | | | | | 200 | | | Table 3 |
| Ruber | R03 | 21 | 19.00 | 0 | 600 | 200-200- | 10,000 | 7 | FIG. 11, |
| | | | | | | 200 | | | Table 3 |
| Ruber | R04 | 37 | 19.00 | 0 | 600 | 200-200- | 10,000 | 12 | FIG. 11, |
| | | | | | | 200 | | | Table 3 |
| Ruber | R05 | 26 | 21.00 | 0 | 600 | 200-200- | 10,000 | 21 | FIG. 11, |
| | | | | | | 200 | | | Table 3 |
| Ruber | R06 | 23 | 20.00 | 0 | 450 | 150-150- | 10,000 | 8 | FIG. 11, |
| | | | | | | 150 | | | Table 3 |
| Ruber | R07 | 19 | 19.00 | 0 | 450 | 150-150- | 10,000 | 3 | FIG. 11, |
| | | | | | | 150 | | | Table 3 |
| Ruber | R08 | 31 | Not | 0 | 400 | 125-125- | 10,000 | 10 | FIG. 11, |
| | | | Determined | | | 75-75 | | | Table 3 |
| Ruber | R09 | 20 | 22.00 | 0 | 400 | 125-125- | 10,000 | 7 | FIG. 11, |
| | | | | | | 75-75 | | | Table 3 |
| Ruber | R10 | 21 | Not | 0 | 400 | 125-125- | 10,000 | 7 | FIG. 11, |
| | | | Determined | | | 75-75 | | | Table 3 |
| Ruber | R11 | 26 | 21.00 | 0 | 400 | 125-125- | 10,000 | 4 | FIG. 11, |
| | | | | | | 75-75 | | | Table 3 |
| Ruber | R12 | 31 | 24.00 | 0 | 600 | 200-200- | 10,000 | 7 | FIG. 11, |
| | | | | | | 200 | | | Table 3 |
| Ruber | R13 | 25 | 28.00 | 0 | 600 | 200-200- | 10,000 | 3 | FIG. 11, |
| | | | | | | 200 | | | Table 3 |
| Ruber | R16 | 31 | 20.00 | 0 | 600 | 200-200- | 10,000 | 14 | FIG. 11, |
| | | | | | | 200 | | | Table 3 |
| Ruber | R18 | 31 | 20.55 | 0 | 600 | 300-150- | 10,000 | 6 | FIG. 11, |
| | | | | | | 150 | | | Table 3 |
| Ruber | R19 | 27 | 21.23 | 0 | 600 | 300-150- | 10,000 | 10 | FIG. 11, |
| | | | | | | 150 | | | Table 3 |
| Ruber | R22 | 25 | 23.44 | 0 | 600 | 300-150- | 10,000 | 7 | FIG. 11, |
| | | | | | | 150 | | | Table 3 |
| Ruber | R23 | 28 | 27.44 | 0 | 600 | 200-200- | 10,000 | 5 | FIG. 11, |
| | | | | | | 200 | | | Table 3 |
| Ruber | R24 | 25 | Not | 0 | 600 | 300-150- | 10,000 | 12 | FIG. 11, |
| | | | Determined | | | 150 | | | Table 3 |
| Ruber | R25 | 29 | 22.66 | 0 | 600 | 300-150- | 10,000 | 11 | FIG. 11, |
| | | | | | | 150 | | | Table 3 |
| Ruber | R38 | 25 | 22.76 | 0 | 600 | 200-200- | 10,000 | 10 | FIG. 12, |
| | | | | | | 200 | | | Table 3 |
| Ruber | R39 | 27 | 22.98 | 0 | 600 | 200-200- | 10,000 | 7 | FIG. 12, |
| | | | | | | 200 | | | Table 3 |
| Ruber | R40 | 30 | 22.77 | 0 | 600 | 200-200- | 10,000 | 8 | FIG. 12, |
| | | | | | | 200 | | | Table 3 |
| Ruber | R41 | 28 | 24.38 | 0 | 600 | 200-200- | 10,000 | 4 | FIG. 12, |
| | | | | | | 200 | | | Table 3 |
| Ruber | R42 | 33 | 24.24 | 0 | 600 | 200-200- | 10,000 | 11 | FIG. 12, |
| | | | | | | 200 | | | Table 3 |
| Ruber | R43 | 34 | 26.23 | 0 | 600 | 200-200- | 10,000 | 7 | FIG. 12, |
| | | | | | | 200 | | | Table 3 |
| Ruber | R44 | 27 | 20.6 | 0 | 600 | 200-200- | 10,000 | 12 | FIG. 12, |
| | | | | | | 200 | | | Table 3 |
| Ruber | R45 | 28 | 17.04 | 0 | 600 | 200-200- | 10,000 | 11 | FIG. 12, |
| | | | | | | 200 | | | Table 3 |
| Ruber | R46 | 34 | 19.05 | 0 | 600 | 200-200- | 10,000 | 8 | FIG. 12, |
| | | | | | | 200 | | | Table 3 |
| Ruber | R47 | 29 | 18.36 | 0 | 600 | 200-200- | 10,000 | 2 | FIG. 12, |
| | | | | | | 200 | | | Table 3 |
| Ruber | R49 | 33 | 19.72 | 0 | 600 | 200-200- | 10,000 | 9 | FIG. 12, |
| | | | | | | 200 | | | Table 3 |
| Ruber | R50 | 28 | 24.84 | 0 | 600 | 200-200- | 10,000 | 2 | FIG. 12, |
| | | | | | | 200 | | | Table 3 |
| Ruber | R51 | 26 | 23.53 | 0 | 600 | 200-200- | 10,000 | 8 | FIG. 12, |
| | | | | | | 200 | | | Table 3 |
| Ruber | R52 | 30 | 23.03 | 0 | 600 | 200-200- | 10,000 | 8 | FIG. 12, |
| | | | | | | 200 | | | Table 3 |
| Ruber | R53 | 25 | 19.03 | 0 | 600 | 200-200- | 10,000 | 7 | FIG. 12, |
| | | | | | | 200 | | | Table 3 |
| Ruber | R54 | 31 | 24.22 | 0 | 600 | 200-200- | 10,000 | 4 | FIG. 12, |
| | | | | | | 200 | | | Table 3 |
| Ruber | R55 | 29 | 20.31 | 0 | 600 | 200-200- | 10,000 | 6 | FIG. 12, |
| | | | | | | 200 | | | Table 3 |
| Ruber | R56 | 27 | 21.3 | 0 | 600 | 200-200- | 10,000 | 3 | FIG. 12, |
| | | | | | | 200 | | | Table 3 |
| Ruber | R57 | 32 | 25.97 | 0 | 600 | 200-200- | 10,000 | 4 | FIG. 12, |
| | | | | | | 200 | | | Table 3 |
| Ruber | R58 | 32 | 25.34 | 0 | 600 | 200-200- | 10,000 | 5 | FIG. 12, |
| | | | | | | 200 | | | Table 3 |
| Ruber | R59 | 27 | 21.8 | 0 | 600 | 200-200- | 10,000 | 8 | FIG. 12, |
| | | | | | | 200 | | | Table 3 |
| Spring | SP02 | 26 | 22.70 | 500 | 150 | 150 | 2,500 | 7 | FIG. 13, |
| | | | | | | | | | Table 3, |
| | | | | | | | | | Table 4 |
| Spring | SP03 | 33 | 23.40 | 500 | 300 | 150-150 | 2,500 | 11 | FIG. 13, |
| | | | | | | | | | Table 3, |
| | | | | | | | | | Table 4 |
| Spring | SP04 | 33 | 19.10 | 500 | 300 | 150-150 | 2,500 | 5 | FIG. 13, |
| | | | | | | | | | Table 3, |
| | | | | | | | | | Table 4 |
| Spring | SP05 | 30 | 21.60 | 500 | 300 | 150-150 | 0 | 11 | FIG. 13, |
| | | | | | | | | | Table 3, |
| | | | | | | | | | Table 4 |
| Spring | SP09 | 29 | 24.10 | 500 | 300 | 150-150 | 0 | 4 | FIG. 13, |
| | | | | | | | | | Table 3, |
| | | | | | | | | | Table 4 |

ii. Aspiration of Small Ovarian Follicles to Retrieve Immature Cumulus Oocyte Complexes

Aspirations were performed 36 hours after the trigger injection (10,000 IU hCG) using a transvaginal ultrasound with a needle guide on the probe to retrieve oocytes for co-culture experiments. Aspiration was performed using ASP medium (Vitrolife®) without follicular flushing using double lumen 19-gauge needles (double lumen needles were selected due to the additional stiffness provided by the second channel inside the needle). Vacuum pump suction (100 mm Hg) was used to harvest follicular contents through the aspiration needle and tubing into a 15 mL round bottom polystyrene centrifuge tube. For the conditions where the final outcome was embryo formation, aspirations were performed 36 hours after trigger injection (10,000 IU hCG) or 48 hours after last rFSH injection for untriggered cycles. Aspiration was performed without follicular flushing using a single lumen 19- or 20-gauge needle with a vacuum pump suction (˜200 mm Hg) used to harvest follicular contents through the aspiration needle and tubing into a 15 mL round bottom polystyrene centrifuge tube. In all cases, rapid rotation of the aspiration needle around its long axis, when the follicle had collapsed, provided a curettage effect to assist the release of COCs into the aspirate fluid. Although follicles were not flushed, the aspiration needle was removed from the subject and flushed frequently throughout the oocyte retrieval procedure to limit clotting and needle blockages.

Follicular aspirates were examined in the laboratory using a dissecting microscope. Aspirates tended to include more blood than in typical IVF follicle aspirations, so were washed with HEPES media (G-MOPS Plus, Vitrolife®) to minimize clotting. Often, the aspirate was additionally filtered using a 70-micron cell strainer (Falcon®, Corning) to improve the oocyte search process. COCs were transferred using a sterile Pasteur pipette to a dish containing LAG Medium (Medicult, CooperSurgical®) until use in the IVM procedure. The number of COCs aspirated was equal to roughly 40% of the antral follicles seen in the subject's ovaries on the start day.

iii. Preparation of Ovarian Supporting Cells (OSCs)

OSCs were created from human induced pluripotent stem cells (hiPSCs) according to transcription factor (TF)-directed protocols described previously. The OSCs were produced in multiple batches and cryopreserved in vials of 120,000 to 150,000 live cells each and stored in liquid nitrogen in CryoStor CS10 Cell Freezing Medium (StemCell Technologies®).

Culture dishes (4+8 Dishes, BIRR) for oocyte maturation experiments were prepared with culture medium and additional constituents in 100 μL droplets under mineral oil the day before oocyte collection. The morning of oocyte collection, cryopreserved OSCs were thawed for 2-3 minutes at 37° C. (in a heated bead or water bath), resuspended in OSC-IVM medium and washed twice using centrifugation and pelleting to remove residual cryoprotectant. Equilibrated OSC-IVM media was used for final resuspension. OSCs were then plated at a concentration of 100,000 OSCs per 100 μL droplet by replacing 50 μL of the droplet with 50 μL of the OSC suspension 2-4 hours before the addition of oocytes to allow for culture equilibration and media conditioning (FIG. 2A).

iv. In Vitro Maturation

COCs were maintained in preincubation LAG Medium (Medicult, CooperSurgical®) at 37° C. for 2-3 hours after retrieval prior to introduction to in vitro maturation conditions. Two different sets of experimental comparisons were performed to address the following goals:

Experiment 1 (OSC activity): The purpose of this comparison was to determine whether the stimulated OSCs were the active ingredient of the co-culture system. For this purpose, medium in experimental and control conditions was prepared by following Medicult manufacturer's recommendations, and further supplemented with androstenedione and doxycycline (both necessary for activation/stimulation of OSCs) in order to compare maturation outcomes with or without OSCs in the same medium formulation (see Table 2 below).

Experiment 2 (OSC clinical relevance): The purpose of this experiment was to compare the efficacy of the OSC-IVM system and the commercially available in vitro maturation system (Medicult IVM).

For this purpose, the Control Group condition was prepared and supplemented by following Medicult manufacturer's recommendations, while medium for OSC-IVM was prepared with all supplements (see Table 2 below).

TABLE 2
Cell culture media conditions

| | Experiment 1: | | Experiment 2: | |
|---|---|---|---|---|
| | OSC Activity | | OSC Clinical Relevance | |
| | Experimental | Control | Experimental | Control |
| | Group | Group | Group | Group |
| IVM medium (Medicult, CooperSurgical ®) | X | X | X | X |
| 75 mIU/mL rFSH (Millipore, F4021) | X | X | X | X |
| 100 mIU/mL rhCG (Sigma, CG10) | X | X | X | X |
| 500 ng/mL Androstenedione (Sigma, A-075I) | X | X | X | |
| 1.0 μ/mL Doxycycline (StemCell Tech., | X | X | X | |
| 100-1047) | | | | |
| 100,000 OSCs per 100 μL droplet | X | | X | |

Subject description (Experiment 1): We collected 132 oocytes from 25 subjects (average age of 25) who underwent abbreviated gonadotropin stimulation, with 49 utilized in OSC-IVM co-culture, and 83 utilized in control culture. Co-culture in the Experimental and Control Conditions was performed in parallel when possible. COCs were distributed equitably when performed in parallel. Equitable distribution means that COCs with distinctly large cumulus masses, small cumulus masses, or expanded cumulus masses were distributed as equally as possible between the two conditions. Other than the selective distribution of the distinct COC sizes, the COCs were distributed as randomly as possible between one to two conditions. Due to the low number of oocytes retrieved per subject in this comparison, it was often not possible to distribute oocytes effectively between conditions simultaneously. COCs were subjected to these in vitro maturation conditions at 37° C. for a total of 24-28 hours in a tri-gas incubator with CO₂ adjusted so that the pH of the bicarbonate-buffered medium was 7.2-7.3 and with the O₂ level maintained at 5%.

Subject description (Experiment 2): For the IVM outcome endpoint, 21 subjects were recruited for the comparison. We collected 143 COCs included in the comparison, allocating 70 utilized in IVM control and 73 utilized in the OSC-IVM condition. Co-culture in the Experimental and Control Conditions was performed in parallel for all subjects. COCs were distributed equitably between the two conditions, as described above. COCs were subjected to these in vitro maturation conditions at 37° C. for a total of 28 hours in a tri-gas incubator with CO₂ adjusted so that the pH of the bicarbonate-buffered medium was 7.2-7.3 and with the O₂ level maintained at 5%. In vitro maturation with subsequent embryo formation was performed to assess developmental competence of the oocytes treated in the OSC-co-culture system in comparison to oocytes treated with commercially available IVM medium. For embryo formation, a small cohort of oocyte donors were recruited and donor sperm was utilized for fertilization. For the embryo outcomes endpoint, six additional subjects were recruited for the comparison. We collected 46 COCs included in the comparison, allocating 21 utilized in Media-IVM control and 25 utilized in the OSC-IVM condition. Co-culture in the Experimental and Control Conditions was performed in parallel. COCs were distributed equitably between the two conditions, as described above. COCs were subjected to these in vitro maturation conditions at 37° C. for a total of 28 hours in a tri-gas incubator with CO₂ adjusted so that the pH of the bicarbonate-buffered medium was 7.2-7.3 and with the O₂ level maintained at 5%. Embryo formation proceeded in parallel, with groups kept separate, with culture proceeding no longer than day 7 post-IVM.

v. Assessment of In Vitro Maturation

Following the end of the 24 to 28 hour in vitro maturation period, COCs were subjected to hyaluronidase treatment to remove surrounding cumulus and corona cells. After hyaluronidase treatment, cumulus cells were banked for future analysis and oocytes were assessed for maturation state according to the following criteria:
GV—presence of a germinal vesicle, typically containing a single nucleolus within the oocyte.
MI—absence of a germinal vesicle within the oocyte and absence of a polar body in the perivitelline space between the oocyte and the zona pellucida.
MII—absence of a germinal vesicle within the oocyte and presence of a polar body in the perivitelline space between the oocyte and the zona pellucida.
   vi. Oocyte Morphology Scoring

Following IVM, oocytes were harvested from culture dishes and stripped of cumulus cells and OSCs, assessed for maturation assessment, then individually imaged using digital photomicrography. After imaging, oocytes were flash frozen in 0.2 mL PCR tubes prefilled with 5 μL DPBS. The images were later scored according to the Total Oocyte Score (TOS) grading system. Oocytes were scored by a single trained embryologist and given a score of −1, 0, 1 for each of the following criteria: morphology, cytoplasmic granularity, perivitelline space (PVS), zona pellucida (ZP) size, polar body (PB) size, and oocyte diameter. Zona pellucida and oocyte diameter were measured using ECHO™ Revolve Microscope software and ImageJ image analysis software (2.9.0/1.53 t). The sum of all categories was taken to give the oocyte a total quality score, ranging from −6 to +6 with higher scores indicating better morphological quality.

vii. Oocyte Disposition Following Morphological Scoring

For oocytes used only for evaluation of oocyte maturation, oocytes were snap frozen following assessment of in vitro maturation and any further morphology scoring. Snap freezing was performed by placing each oocyte in a 0.25 mL PCR tube with 5 μL DPBS. After capping the tube, it was submerged in liquid nitrogen until all bubbling ceased. Then the PCR tube was stored at −80° C. for future molecular analysis.

For oocytes used to create embryos, matured oocytes were immediately utilized for intracytoplasmic sperm injection (ICSI) and subsequent embryo formation to the blastocyst stage. No oocytes from this study were utilized for transfer, implantation, or reproductive purposes.

viii. In Vitro Fertilization and Embryo Culture

A cohort of six subjects was utilized for in vitro maturation and subsequent embryo formation. The COCs from these subjects were subjected to the conditions used in Experiment 2 (treatment with OSC co-culture with all adjuvants versus commercially available IVM treatment as the control). All COCs were cultured for 28 hours then denuded and assessed for MII formation and micrographed. Individual oocytes in each condition were injected with sperm (intracytoplasmic sperm injection (ICSI) on day 1 post-retrieval. After ICSI, the oocytes were cultured in a medium designed for embryo culture (Global Total, CooperSurgical®, Bedminster, NJ) at 37° C. in a tri-gas incubator with CO₂ adjusted so that the pH of the bicarbonate-buffered medium was 7.2-7.3 and with the O₂ level maintained at 5%. The following day they were assessed for fertilization 12 to 16 hours post-ICSI, and oocytes with one or two pronuclei were cultured until day 3. Cleaved embryos underwent laser-assisted zona perforation and were allowed to develop until the blastocyst stage. Blastocysts were scored according to the Gardner scale then underwent trophectoderm biopsy for preimplantation genetic testing for aneuploidy (PGT-A) and cryopreservation if deemed high quality, i.e., greater than or equal to a 3CC rating.

Trophectoderm biopsies were transferred to 0.25 mL PCR tubes and sent to a reference laboratory (Juno Genetics®, Basking Ridge, NJ) for comprehensive chromosomal analysis using a single nucleotide polymorphism (SNP) based next generation sequencing (NGS) of all 46 chromosomes (pre-implantation genetic testing for aneuploidy, PGT-A).

ix. Data Analysis and Statistics

Oocyte maturation outcome data was analyzed using Python statistical packages pandas (1.5.0), scipy (1.7.3), and statsmodels (0.13.2). Maturation percentages by donor group were analyzed using linear regression as functions of the IVM environment (OSC-IVM or Media control). t-test statistics were computed comparing cell line incubation outcomes versus media control, then used to calculate p-values. Bar graphs depict mean values for each population and error bars represent standard error of the mean (SEM).

### Example 6. HiPSC-Derived OSCs Effectively Promote Human Oocyte Maturation Following Co-Culture System

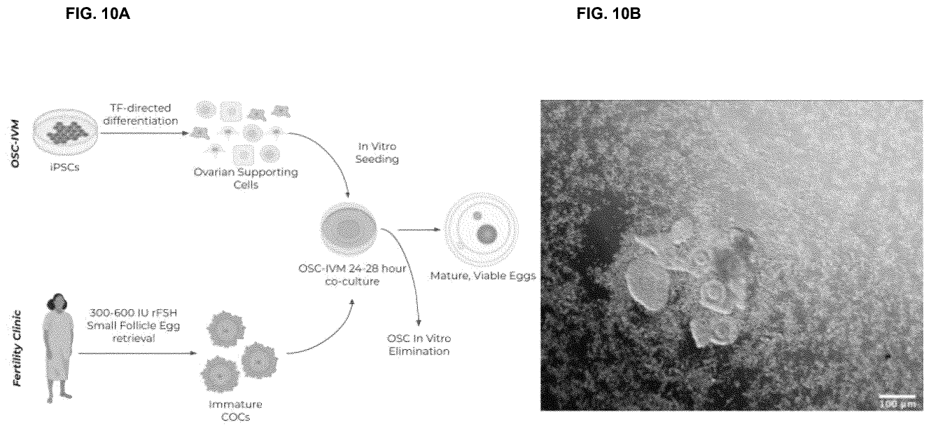

In order to obtain immature COCs for IVM, we utilized similar protocols to previous studies for IVM, truncated IVF or hCG primed-IVM, namely 3-4 days of minimal gonadotropin stimulation and most often an hCG trigger. This abbreviated stimulation program, particularly when hCG was included, yielded a mixed cohort of oocytes that were mostly immature (GV and MI), but expanded cumulus COCs were obtained as well, which may have contained MII oocytes. Oocyte donor demographics and treatment regimens are shown in Table 3 for each experimental group. Overall, the results demonstrate we were able to retrieve oocytes from non-polycystic ovarian syndrome (non-PCO/PCOS) donors, albeit at a lower yield than traditional controlled ovarian hyperstimulation cycles. In Experiment 1, oocytes from each donor were allocated to either the control IVM or OSC-IVM arm. Age, body mass index (BMI) and total COCs retrieved did not significantly differ between groups in Experiment 1. For Experiment 2, the control and OSC-IVM arms for both endpoints contained identical donor groups as oocytes were split equally between culture conditions for each donor. Age and BMI significantly differed in Experiment 2 compared to Experiment 1, and total COCs retrieved per donor was lower but not significantly. A schematic of the OSC-IVM condition is shown in FIG. 10A, with a representative image of the OSC co-culture seen in FIG. 10B.

TABLE 3
Donor demographic and stimulation characteristics

| | Experiment 1: | | Experiment 2: | |
|---|---|---|---|---|
| | OSC Activity | | OSC Clinical Relevance | |
| | Experimental | Control | | Embryo |
| | Group | Group | IVM Only | Formation |
| Number of Oocyte | 12 | 18 | 21 | 6 |
| Donors Included | | | | |
| Oocyte Donor Age | 26 ± 0.94 | 24 ± 1.1 | 29 ± 0.63 | 30.2 ± 1.07 |
| (Avg ± SEM) | | | | |
| Oocyte Donor BMI | 34 ± 4.7 | 33 ± 3.8 | 22 ± 0.58 | 24.9 ± 2.84 |
| (Avg ± SEM) | | | | |
| Total rFSH used | 325-600 | 325-600 | 600 | 5 days |
| for stimulation (IU) | | | | Clomid |
| | | | | plus |
| | | | | 150-300 |
| hCG Trigger | 10,000 | 10,000 | 10,000 | 0 IU or |
| utilized (IU) | | | | 2,500 IU |
| COCs obtained | 7.8 ± 0.98 | 7.7 ± 1.03 | 7.0 ± 0.64 | 7.7 ± 1.20 |
| (Avg ± SEM) | | | | |

We have previously demonstrated that hiPSC-derived OSCs are predominantly composed of granulosa-like cells and ovarian stroma-like cells. In response to hormonal stimulation treatment in vitro, these OSCs produce growth factors and steroids necessary for interaction with oocytes and cumulus cells. To investigate whether hiPSC-derived OSCs are functionally capable of promoting human oocyte maturation in vitro, we established a co-culture system of these cells with freshly retrieved cumulus enclosed oocytes and assessed maturation rates after 24-28 hours (see Materials and Methods, Experiment 1). In this comparison, due to low numbers of retrieved oocytes per donor, we were unable to consistently split oocytes between both conditions simultaneously, therefore each group contains oocytes from predominantly non-overlapping donor groups and pairwise comparisons are not utilized. Strikingly, we observed significant improvement (˜1.5×) in maturation outcomes for oocytes that undergo IVM with OSCs (FIG. 11A) compared to control. More specifically, the OSC-IVM group yielded a maturation rate of 68%±6.83% SEM versus 46%±8.51% SEM in the Media Control (FIG. 11A, p=0.02592, unpaired t-test). The maturation rate for OSC-IVM compared to control was statistically significant. These results support functional activity of hiPSC-derived OSC in in vitro co-culture systems demonstrated by the significantly higher oocyte maturation rates.

We next examined whether hiPSC-derived OSCs would also affect the outcome of the Total Oocyte Score (TOS). Interestingly, the assessment scores (FIG. 11B) were not statistically significantly different for the two groups (unpaired t-test, p=0.2909), indicating that the mature MII oocytes outcome was of equivalent morphological quality between the two IVM conditions. Altogether, these data indicate that OSC co-culture improve maturation without a detrimental effect on morphological quality of human oocytes, and highlights the potential for the use of hiPSC-derived OSCs as a high performing system for cumulus enclosed oocyte IVM.

### Example 7. Oocyte Maturation Rates in OSC-IVM Outperforms Commercially Available IVM System

To further examine the potential of using OSC-IVM as a viable system to mature human oocytes in a clinical setting, we compared our OSC co-culture system against a commercially available IVM standard. The commercially available IVM standard was utilized as described in its clinical instructions for use, with no modification (Medicult IVM). We performed a sibling oocyte study comparing the MII formation rate and oocyte morphological quality after 28 hours of in vitro maturation in both systems (Materials and Methods, Experiment 2). Notably, OSC-IVM yielded ˜1.6×higher average MII formation rate with 68%±6.74% of mature oocytes across donors compared to 43%±7.90% in the control condition (FIG. 12A, p=0.0349, paired t-test). The maturation rate for OSC-IVM compared to the commercial IVM control was statistically significant. Similar to previous observations, co-culture with hiPSC-derived OSCs did not affect oocyte morphological quality between groups as measured by TOS, indicating equivalent oocyte visual morphological characteristics (FIG. 12B, p=0.9420, unpaired t-test). These results show that OSC-IVM significantly outperformed the commercially available IVM culture medium in MII formation rate with no apparent detriment to oocyte morphological quality, pointing to a beneficial application for human IVM.

### Example 8. Cumulus Enclosed Immature Oocytes from Abbreviated Gonadotropin Stimulation Matured by OSC-IVM are Developmentally Competent for Embryo Formation

We sought to investigate the developmental competency of oocytes treated in the OSC-IVM system, by assessing euploid blastocyst formation, compared to the commercially available IVM control. Utilizing a limited cohort of six subjects who underwent abbreviated stimulation (see Materials and Methods Experiment 2, Tables 2 and 3) we investigated whether OSC-IVM treated oocytes were capable of fertilization, cleavage, and formation of euploid blastocysts. We compared these embryo outcomes to those found from oocytes treated in the commercially available IVM medium. OSC-IVM yielded ˜1.2X higher average MII formation rate with 60%±15.4% of mature oocytes across donors compared to 52%±8% in the control condition (FIG. 13A, Table 4). Mature oocytes in both treatment groups were subjected to ICSI and fertilized oocytes were cultured until Day 7 of development. OSC-assisted Mils demonstrate a trend towards improved fertilization, cleavage, blastocyst and usable quality blastocyst formation rates as a proportion of the input COC number (52%, 52%, 40%, and 28%) compared to the commercial IVM control (38%, 38%, 24%, and 19%) (FIG. 13A, Table 4). When examined on an incremental basis, OSC-IVM oocytes fertilize and form blastocysts at an improved rate, while cleavage of fertilized oocytes is similar to the commercial IVM control. Overall, in both conditions we find that all oocytes that fertilized subsequently cleaved. Strikingly, PGT-A results show that of the blastocysts of transferable quality generated by OSC-IVM, 100% are euploid versus 25% in the commercial IVM system. While these results are not statistically significant, likely due to the small underpowered sample size for each group, these findings demonstrate that OSC-IVM generates healthy matured oocytes with high quality developmental competency. These results additionally demonstrate OSC-IVM is capable of producing healthy, euploid embryos from abbreviated stimulation cycles at a higher rate than the commercially available IVM condition, highlighting the clinical relevance of this novel system for IVM ART practice.

TABLE 4
OSC-IVM oocytes are developmentally competent for euploid embryo formation

| Developmental | OSC-IVM (COCs = 25) | Commercial IVM (COCs = 21) | |
|---|---|---|---|
| Step | n (% incremental, % of COCs) | n (% incremental, % of COCs) | p-value |
| Maturation Rate | 15 (60% ± 15.4%, 60% ± 15.4%) | 11 (52% ± 8%, 52% ± 8%) | 0.351 |
| Fertilization Rate | 13 (87% ± 7%, 52% ± 14%) | 8 (73% ± 16%, 38% ± 11%) | 0.263 |
| Cleavage Rate | 13 (100% ± 0%, 52% ± 14%) | 8 (100% ± 0%, 38% ± 11%) | 0.263 |
| Blastocyst Rate | 10 (77% ± 20%, 40% ± 17%) | 5 (63% ± 21%, 24% ± 11%) | 0.257 |
| Usable Quality | 7 (70% ± 21%, 28% ± 16%) | 4 (80% ± 13%, 19% ± 8%) | 0.324 |
| Blastocyst Rate | | | |
| High Quality | 7 (100% ± 0%, 28% ± 16%) | 1 (25% ± 25%, 5% ± 3%) | 0.081 |
| Euploid | | | |
| Blastocyst Rate | | | |

### Example 9. Materials and Methods for Examples 10-12

We have demonstrated that human ovarian support cells (OSCs) generated from human induced pluripotent stem cells (hiPSCs) exhibit the ability to recapitulate dynamic ovarian function in vitro. Here we investigate the utilization of these OSCs as a co-culture system to better mimic the ovarian environment in vitro and promote IVM to rescue denuded immature oocytes derived from conventional gonadotropin stimulated cycles. We find that OSC-IVM significantly improves oocyte maturation rates compared to spontaneous maturation in media matched controls. Additionally, oocytes matured in combination with OSC-IVM are transcriptionally more similar to conventional IVF metaphase II (MII) oocytes than oocytes that had spontaneously matured in media controls. Together, these findings demonstrate a novel approach to improve the outcome of matured MII oocytes in modern IVF practice by leveraging an optimized IVM system that better mimics the ovarian environment in vitro.

Specifically, to determine if rescue in vitro maturation (IVM) of human oocytes can be improved by co-culture with ovarian support cells (OSCs) derived from human induced pluripotent stem cells (hiPSCs), fertility patients undergoing conventional ovarian stimulation donated denuded immature germinal vesicle (GV) and metaphase I (MI) oocytes for research, which were allocated between either the control or intervention.

Oocyte donors between the ages of 25 to 45 years old donated immature oocytes under informed consent, with no additional inclusion criteria. The 24-28 hour OSC-IVM culture condition was composed of 100,000 OSCs in suspension culture with human chorionic gonadotropin (hCG), recombinant follicle stimulating hormone (rFSH), androstenedione and doxycycline supplementation. The IVM control lacked OSCs and contained the same supplementation.

Primary endpoints consisted of MII formation rate and morphological quality assessment. Additionally, metaphase spindle assembly location and oocyte transcriptomic profiles were assessed compared to in vivo matured oocyte controls. OSC-IVM resulted in a statistically significant improvement in MII formation rate compared to the Media-IVM control. Oocyte morphological quality between OSC-IVM and the Media-IVM control did not significantly differ. OSC-IVM resulted in MII oocytes with no instance of spindle absence and no significant difference in position compared to in vivo matured MII controls. OSC-IVM treated MII oocytes display a transcriptomic maturity signature significantly more similar to IVF-MII controls than the Media-IVM control MII oocytes.

i. Collection of Immature Oocytes

Forty-seven oocyte donor subjects were enrolled in the study using informed consent (IRB #20222213, Western IRB). Subject ages ranged between 25 and 45 years of age, with an average age of 35. Oocytes were retrieved at several in vitro fertilization and egg freezing clinics in New York City (IRB #20222213, Western IRB). Fertility patients providing discarded immature oocytes had signed informed consents, provided by the clinic, permitting their use for research purposes. Patients underwent typical age-appropriate controlled ovarian hyperstimulation using gonadotropin releasing hormone (GnRH) analogs (agonist or antagonist) or injections with recombinant or highly purified urinary gonadotropins (recombinant FSH, human menopausal gonadotropins) followed by an ovulatory trigger (human Chorionic Gonadotropin (hCG) or GnRH agonist). 34-36 hours following the trigger injection(s), oocytes were retrieved from the patient under conscious sedation using standard clinical procedures.

Retrieved oocytes were exposed to hyaluronidase briefly then adherent cumulus cells were mechanically removed by repeatedly drawing up and expelling each cumulus-oocyte complex with a small-bore pipette. Denuded oocytes were assessed for maturation by observation of a polar body or a germinal vesicle. Immature oocytes (GV or MI), which would usually be discarded, were instead allocated to our research study. All immature oocytes retrieved from the clinic each day were pooled and were placed in LAG Medium (Medicult, CooperSurgical®) in a 5 mL round-bottom tube that was transferred from the clinic to our research laboratory in a 37° C. transport incubator.

For some experiments, immature (GV and MI) oocytes from similar IVF and egg freezing cycles were vitrified and stored at the clinics. Cryopreserved oocytes were transported from the clinic to our laboratory in liquid nitrogen and stored until use. Oocytes were then thawed using the standard Kitazato protocol for vitrified or slow frozen oocytes (Vitrolife®, USA), evaluated for maturation status as GV or MI, and used for comparisons of in vitro maturation conditions.

A limited number of MII oocytes obtained from conventional controlled ovarian hyperstimulation, which were previously banked for research purposes, were provided as controls for this study (IVF-MII). These oocytes were transferred to our laboratory from the tissue repository and thawed using either the standard Kitazato protocol for vitrified oocytes (Kitazato™, USA) or slow freeze-thaw protocol for previously slow frozen oocytes (Vitrolife®, USA) and utilized for live fluorescent imaging and transcriptomic analysis.

ii. Preparation of Ovarian Supporting Cells (OSCs)

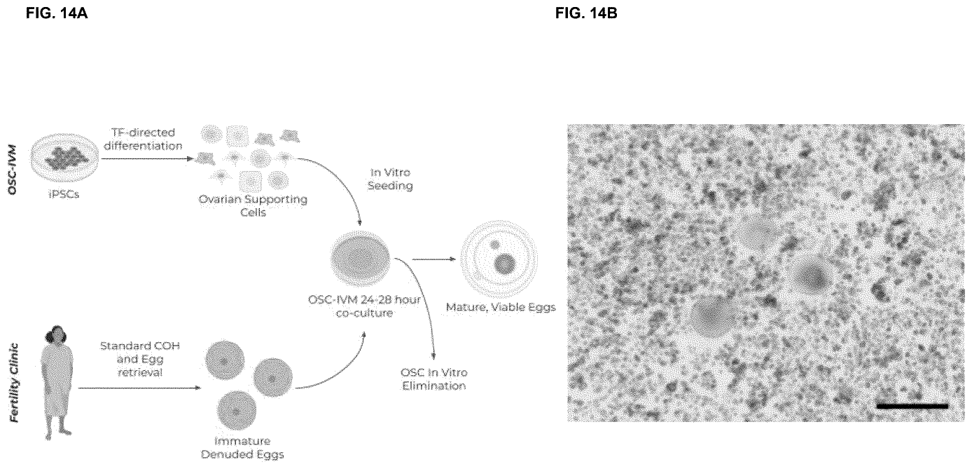

Human induced pluripotent stem cell (hiPSC) derived OSCs were created according to transcription factor (TF)-directed protocols described previously. OSCs were produced in multiple batches and cryopreserved in vials of 120,000 to 150,000 cells each and stored in the vapor phase of liquid nitrogen in CryoStor™ CS10 Cell Freezing Medium (StemCell Technologies®). Culture dishes (4+8 Dishes, BIRR) for oocyte maturation experiments were prepared with culture medium and additional constituents in 100 μL droplets under mineral oil (LifeGuard, LifeGlobal Group®) the day before oocyte collection. The morning of oocyte collection, cryopreserved OSCs were thawed for 2-3 minutes at 37° C. (in a heated bead or water bath), resuspended in OSC-IVM medium and washed twice using centrifugation and pelleting to remove residual cryoprotectant. Equilibrated OSC-IVM medium was used for final resuspension. OSCs were then plated at a concentration of 100,000 OSCs per 100 μL droplet by replacing 50 μL of the droplet with 50 μL of the OSC suspension 2-4 hours before the addition of oocytes to allow for culture equilibration and culture medium conditioning (FIG. 14A). OSCs were cultured in suspension culture surrounding the denuded oocytes in the microdroplet under oil. IVM culture proceeded for 24 to 28 hours, after which oocytes were removed from culture, imaged, and harvested for molecular analysis.

iii. In Vitro Maturation

Immature oocytes were maintained in preincubation LAG Medium (Medicult, CooperSurgical®) at 37° C. for 2-3 hours after retrieval prior to introduction to in vitro maturation conditions (either Media-IVM or OSC-IVM).

A single experimental condition was examined:

Experiment (OSC activity): The purpose of this comparison was to determine whether the stimulated OSCs were the active ingredient or contributor to the co-culture system. For this purpose, medium in both experimental and control condition was prepared by following Medicult manufacturer's recommendations, and further supplemented with androstenedione and doxycycline (both necessary for activation/stimulation of OSCs) in order to compare maturation outcomes with or without OSCs in the same medium formulation (see Table 5 below).

TABLE 5
Cell culture media conditions

| | Experiment: | |
|---|---|---|
| | OSC activity | |
| | OSC- | Media- |
| | IVM | IVM |
| IVM medium (Medicult, CooperSurgical) | X | X |
| 75 mIU/mL of recombinant FSH (Millipore, F4021) | X | X |
| 100 mIU/mL of recombinant hCG (Sigma, CG10) | X | X |
| 500 ng/mL Androstenedione (Sigma, A-075I) | X | X |
| 1.0 μg/mL Doxycycline (StemCell Tech., 100-1047) | X | X |
| 100,000 OSCs per 100 μL droplet | X | |

Donated oocytes were retrieved from 56 patients and pooled into 29 independent cultures, totaling 141 oocytes, with 82 oocytes utilized in OSC-IVM and 59 oocytes utilized in Media-IVM. Culture in the Experimental and Control Conditions was performed in parallel when possible. Immature oocytes from each donor pool were distributed equitably between two conditions at a time, with no more than 15 oocytes per culture at a time. Specifically, immature oocytes (GV and MI) were distributed as equally and randomly as possible between the two conditions. Due to low and highly variable numbers of available immature oocytes which were provided as discard donation, both conditions often could not be run in parallel from the same oocyte donation source often. Immature oocytes were subjected to in vitro maturation at 37° C. for a total of 24-28 hours in a tri-gas incubator with CO₂ adjusted so that the pH of the bicarbonate-buffered medium was 7.2-7.3 and with the O₂ level maintained at 5%.

iv. Assessment of In Vitro Maturation

At the end of the in vitro culture, oocytes were harvested from culture dishes and mechanically denuded and washed of any residual OSCs. Oocytes were then individually assessed for maturation state according to the following criteria:
GV—presence of a germinal vesicle, typically containing a single nucleolus within the oocyte.
MI—absence of a germinal vesicle within the oocyte and absence of a polar body in the perivitelline space between the oocyte and the zona pellucida.
MII—absence of a germinal vesicle within the oocyte and presence of a polar body in the perivitelline space between the oocyte and the zona pellucida.

Following assessment of in vitro maturation and morphology scoring, oocytes were individually imaged using digital photomicrography and if required, examined by fluorescent imaging for the second meiotic metaphase spindle. No oocytes from this study were utilized for embryo formation, transfer, implantation, or reproductive purpose.

v. Oocyte Morphology Scoring

Oocytes harvested post-IVM were individually imaged using digital photomicrography on the ECHO™ Revolve inverted fluorescent microscope using phase contrast imaging. The images were later scored according to the Total Oocyte Score (TOS) grading system. A single trained embryologist was blinded and oocytes were given a score of −1, 0, 1 for each of the following criteria: morphology, cytoplasmic granularity, perivitelline space (PVS), zona pellucida (ZP) size, polar body (PB) size, and oocyte diameter. Zona pellucida and oocyte diameter were measured using ECHO™ Revolve Microscope software and the image analysis software FIJI (2.9.0/1.53t). The sum of all categories was taken to give the oocyte a total quality score, ranging from −6 to +6 with higher scores indicating better morphological quality.

vi. Examination of the Second Meiotic Metaphase Spindle and its Position Relative to the Polar Body

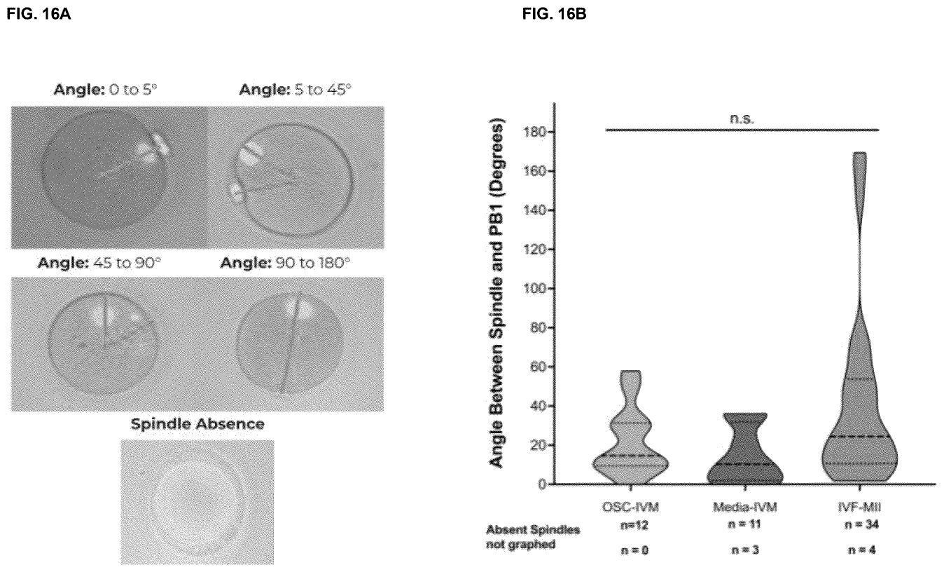

Previously vitrified denuded immature oocytes were thawed and equitably distributed across OSC-IVM and Media-IVM conditions before being cultured for 28 hours. Additional donated MII oocytes were collected and stained to visualize the microtubules of the meiotic spindle apparatus by fluorescent microscopy as an IVF control (IVF-MII) (FIG. 16A-B). MII oocytes were incubated in 2 μM of an alpha-tubulin dye (Abberior™ Live AF610) for one hour in the presence of 10 μM verapamil (Abberior™ Live AF610). Spindle position was then visualized using fluorescence microscopy (ECHO™ Revolve microscope, TxRED filter block EX:560/40 EM:630/75 DM:585). The angle of the first polar body and spindle apparatus in the IVM oocytes was determined (with the vertex at the center of the oocyte) using FIJI software. This measurement was also made on the cohort of IVF MII oocytes (n=34) as a control reference population.

vii. Cryopreservation of Oocytes for Subsequent Molecular Analyses

Following the completion of morphological examination, oocytes were individually placed in 0.25 mL tubes containing 5 μL Dulbecco's Phosphate Buffered Saline (DPBS) and snap frozen in liquid nitrogen. After the cessation of nitrogen bubble formation the tubes were stored at −80° C. until subsequent molecular analysis.

viii. Single Oocyte Transcriptomics Library Preparation and RNA Sequencing

Libraries for RNA sequencing were generated using the NEBNext™ Single Cell/Low Input RNA Library Prep Kit for Illumina® (NEB #E6420) in conjunction with NEBNext™ Multiplex Oligos for Illumina® (96 Unique Dual Index Primer Pairs) (NEB #E6440S), according to the manufacturer's instructions. Briefly, oocytes frozen in 5 μL DPBS and stored at −80° C. were thawed and lysed in lysis buffer, then RNA was processed for reverse transcriptase and template switching. cDNA was PCR amplified with 12-18 cycles, then size purified with KAPA™ Pure Beads (Roche). cDNA input was normalized across samples. Following fragmentation and end prep, NEBNext™ Unique Dual Index Primer Pair adapters were ligated, and samples were enriched using 8 cycles of PCR. Libraries were cleaned up with KAPA™ Pure Beads, quantified using Quant-iT PicoGreen dsDNA Reagent and Kit (Invitrogen), then an equal amount of cDNA was pooled from each oocyte library. The pool was subjected to a final KAPA™ Pure bead size selection if required and quantified using Qubit dsDNA HS kit (Invitrogen). After verification of library size distribution (˜325 bp peak) using Bioanalyzer HS DNA kit (Agilent), the library pool was subjected to RNA sequencing analysis using the MiSeq Micro V2 (2×150 bp) or MiSeq V2 (2×150 bp) kit on an Illumina® MiSeq according to the manufacturer's instructions.

ix. Oocyte Transcriptomics Data Analysis

Illumina® sequencing files (bcl-files) were converted into fastq read files using Illumina® bcl2fastq (v2.20) software deployed through BaseSpace using standard parameters for low input RNA-seq of individual oocytes. Low input RNA-seq data gene transcript counts were aligned to Homo sapiens GRCH38 (v 2.7.4a) genome using STAR (v 2.7.10a) to generate gene count files and annotated using ENSEMBL. Gene counts were combined into sample gene matrix files (h5). Computational analysis was performed using data structures and methods from the Scanpy (v 1.9.1) package as a basis. Gene transcript counts were normalized to 10,000 per sample and log (In) plus 1 transformed. Principal component analysis was performed using Scanpy package methods focusing on 30 PCA components. Integration and project (batch) correction was performed using BBKNN. Projection into two dimensions was performed using the Uniform Manifold Approximation and Projection (UMAP) method. Cluster discovery was performed with the Ledien methods with resolution 0.5.

To define the expected transcriptomic profile for normal MII oocytes we used the donated cohort of in vivo matured IVF-MII samples (n=34) as a reference point and compared this reference set to subsets of the post-IVM GV cells using differential gene expression. The top 50 differentially expressed genes were collected for each comparison using both the Wilcoxon ranked sum test and the cosine similarity-based marker gene identification (COSG) method. No other MI or MII oocyte sets were used as reference points, as these marker genes were developed to ensure minimal bias for other MII transcriptomic profiling. This method generated the failed-to-mature GV and IVF MII signature marker gene expression profiles. Cells were scored for each marker gene set using Scanpy gene marker scoring methods.

To visualize our cells in signature marker space we plotted the marker scores as a two-dimensional space. We then manually divided the space into quadrants based on morphological maturation outcomes and Leiden clusters. Clusters are annotated taking into consideration their distribution in score space and presence in each quadrant correlating their IVM maturation outcome and whole transcriptomic profiles.

x. Data Analysis and Statistics

Oocyte maturation outcome data was analyzed using Python statistical packages pandas (1.5.0), scipy (1.7.3), and statsmodels (0.13.2). Maturation percentages by donor group were analyzed using linear regression as functions of the IVM environment as OSC-IVM or Media-IVM. t-test statistics were computed comparing OSC-IVM versus Media-IVM, then used to calculate p-values using Welch's correction for unequal variance. One way ANOVA was utilized for comparisons of more than two groups for spindle apparatus location analysis. Chi-squared analysis was utilized for comparison of the leiden group population make up in transcriptomic analysis for the three sample conditions. Bar graphs depict mean values for each population and error bars represent standard error of the mean (SEM).

### Example 10. HiPSC-Derived OSCs Effectively Promote Human Oocyte Maturation Following Co-Culture System with Denuded Oocytes

We have previously demonstrated that hiPSC-derived OSCs are predominantly composed of granulosa-like cells and ovarian stroma-like cells. In response to hormonal stimulation treatment in vitro, namely FSH, these OSCs produce growth factors and steroids, and express adhesion molecules necessary for interaction with oocytes and cumulus cells. To investigate whether hiPSC-derived OSCs are functionally capable of promoting human oocyte maturation in vitro, as an approach to rescue immature denuded oocytes, we established a co-culture system of these cells with freshly retrieved denuded immature oocytes and assessed maturation rates after 24-28 hours (FIG. 14).

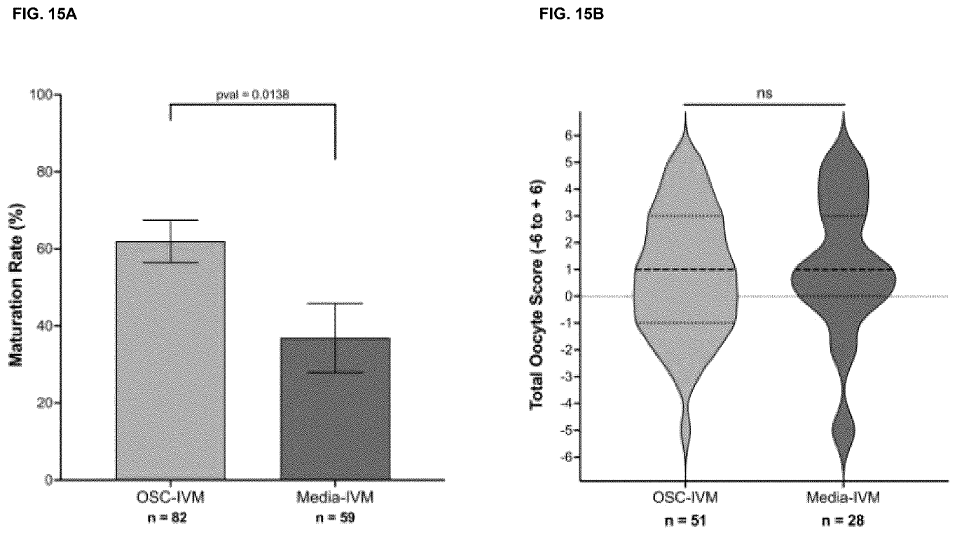

We first examined whether OSC-IVM affected the rate of maturation of denuded oocytes compared to oocytes kept in the Media-IVM condition containing the same culture medium and all supplements but no OSCs, with maturation rates determined per oocyte culture group for each condition. Strikingly, we observed significant improvement in maturation outcome rates (˜1.7×) for oocytes that underwent IVM with OSCs. Specifically, the OSC-IVM group yielded a maturation rate of 62%±5.57% SEM versus 37%±8.96% SEM in the Media-IVM (FIG. 15A, p=0.0138, unpaired t-test). We additionally scored the morphological quality of MII oocytes obtained in both IVM conditions by assessing the Total Oocyte Score (TOS). We found no significant difference between the two groups (FIG. 15B, p=0.5725, unpaired t-test), suggesting that in vitro maturation of denuded oocytes is not affecting the morphological features of Mils. Altogether, these data indicate that OSC co-culture increases the ratio of oocyte maturation, compared to spontaneous maturation observed in the control IVM media, without a detrimental effect on morphological quality of human oocytes, and highlights the potential for the use of hiPSC-derived OSCs for rescuing immature denuded oocytes from IVF procedures.

### Example 11. OSC-IVM Promotes High Quality Assembly of the Second Meiotic Spindle Apparatus in IVM Oocytes

Second meiotic spindle assembly, more specifically both the presence of and the angle of the spindle relative to PB1, has been implicated in previous studies as a key indicator of oocyte quality in relation to fertilization and developmental competence, with the presence of a spindle with a smaller angle relative to the PB1 as an indicator of improved quality. We sought to determine the relative position of the second meiotic spindle apparatus and first polar body in OSC-treated oocytes in comparison to MII oocytes retrieved from IVF cycles (IVF-MII), as a comparative measure of oocyte quality (FIG. 16). We also included as a control the oocytes that spontaneously matured in the Media-IVM condition (FIG. 16A). We found that the spindle angle was not significantly different between conditions (MII OSC-IVM: 22°±5.2 SEM; MII Media-IVM: 15°±5.7 SEM; IVF-MII: 41°±8.3 SEM; p=0.1155; ANOVA), suggesting that in vitro maturation of denuded oocytes does not impair spindle position. Interestingly, the only condition in which we did not observe instances of spindle absence was the condition containing MII oocytes from OSC-IVM (FIG. 16B). More studies are needed to validate the relevance of this observation, but it is likely to be an indication of formation of high-quality oocytes. Altogether, these results indicate that MII oocytes matured in vitro in combination with OSCs hold equivalent spindle angle values to MII oocytes directly retrieved from IVF procedures, suggesting that IVM applied to rescue denuded immature oocytes is not detrimental to oocyte quality based on this parameter.

### Example 12. OSC-IVM Promotes Maturation of Mil Oocytes with High Transcriptomic Similarity Compared to In Vivo Matured Mil Oocytes

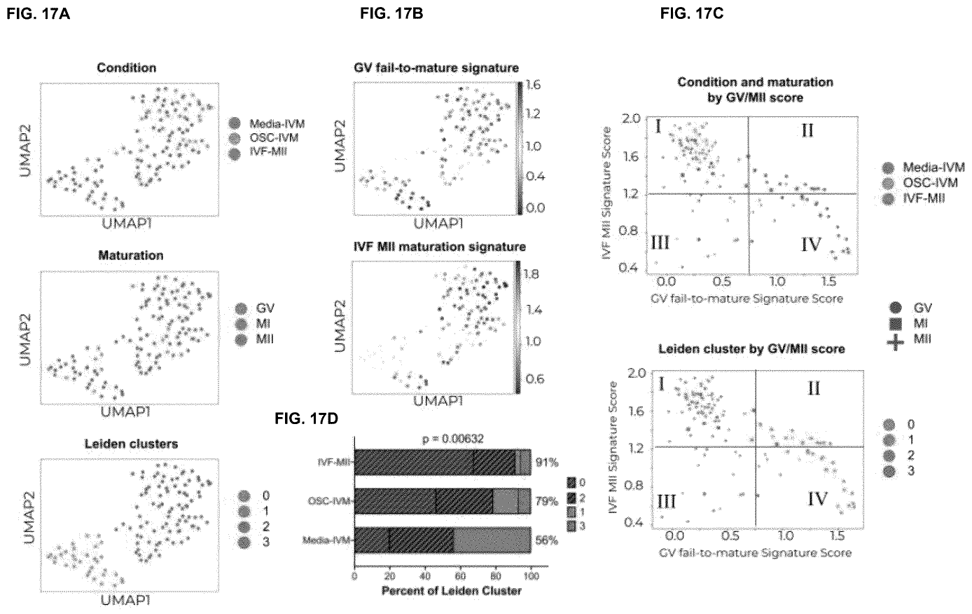

To further compare the quality and maturation of OSC-IVM oocytes relative to a cohort of IVF-MII control oocytes and the Media-IVM oocytes, we performed single oocyte transcriptomic analysis. Transcriptomic analyses provide a global view of oocyte gene expression, providing a strong representation of their cellular state, function, and general attributes. We started by combining our datasets that included: 1) denuded immature oocytes after 24-28 hours in co-culture with OSC (OSC-IVM), 2) denuded immature oocytes kept in the in vitro maturation media control (Media-IVM), and 3) MII oocytes retrieved from regular IVF cycles (IVF-MII). We next generated UMAP plots and annotated individual oocytes by Condition (OSC-IVM, Media-IVM, and IVF-MII) and Maturation outcome (GV, MI, MII) (FIG. 17A). From this analysis, we observed that maturation state was the main driver of oocyte separation in whole transcriptomic space, suggesting that transcriptional profile is a good predictor of oocyte maturation state. MII oocytes project predominantly into the large cluster on the upper right half of the plot (FIG. 17A Maturation). GV oocytes project predominantly into a smaller cluster on the lower left half of the plot. Hence the separation in the UMAP is a combination of the two projected dimensions. As expected, MII oocytes retrieved from IVF (IVF-MII) show close grouping together with MII from both the OSC-IVM, as well as Media-IVM. Similarly, GVs from OSC-IVM and Media-IVM show close distance among each other and apart from the MII oocytes. In contrast, MI oocytes were scattered among both groups, likely a consequence of being an intermediate maturation state and being present in very low numbers in comparison with the other two maturation states (GVs and Mils).

We next generated a reference transcriptomic signature of conventionally matured MII oocytes to assess the quality of Mils rescued/matured in vitro. To set a standard, we used MII oocytes retrieved from conventional ovarian hyperstimulation IVF samples (IVF-MII) to create a gene score for IVF MII maturation signature. In parallel, we used the stalled GVs resultant from IVM conditions (OSC-IVM and Media-IVM) to generate a gene score for IVM GV failed maturation signature (FIG. 17B). These two gene signatures were utilized to capture a relative positive control of IVM, namely an IVF-like successful maturation outcome, as well as a negative control of IVM, namely oocytes that arrest as GVs.

To better understand transcriptomic nuances amongst the mature MII oocytes, we used the Leiden algorithm to further subcluster our samples into groups sharing closer transcriptomic profiles. We identified three clusters (0, 2, and 3) within the MII oocytes population, and one cluster (1) comprised mostly GVs. As expected, the GV maturation signature was strongly represented in cluster 1. Similarly, the MII maturation signature included Mils from both IVF and IVM, and it was more overrepresented in clusters 0 and 2. As such, we designate cluster 1 as representing the (GV) failed maturation transcriptomic profile, while clusters 0 and 2 represent a profile similar to the IVF MII maturation transcriptomic profile. Interestingly, cluster 3 shows lower expression for both the IVF MII and IVM GV failed maturation signatures. This could indicate a transitional state between immature and mature development in which neither signature is highly upregulated, or could result from cell activity stasis, shutdown, or oocyte stalling.

In FIG. 17C, we assess the quality of individual oocytes relative to our IVF MII maturation signature (y-axis), as well as the IVM GV failed maturation signature (x-axis). For visual clarity we divide our signature dimension plot into labeled quadrants which help denote the separation between classification groups. As expected, we observe that most of the oocytes morphologically classified as GVs clustered in the lower right quadrant (IV), holding a high score for GV failed maturation signature along with a low score for IVF MII maturation signature. In contrast, individual oocytes from the IVF-MII condition clustered together (˜91%) in the upper left quadrant (I), holding a high score for MII maturation signature and a low score for GV failed maturation signature. Strikingly, OSC-IVM Mils (blue cross) were found mostly (˜79%) in the upper left quadrant (I) along with the IVF-MII oocytes, suggesting a strong transcriptomic similarity between these two groups. In contrast, Mils from the Media-IVM were often (˜46%) located on the lower left quadrant (III) depicting a low score for both MII maturation signature and GV maturation signature. Interestingly, this lower left quadrant (III) comprises in its majority cells derived from cluster 3, which despite their weak MII maturation signature, were morphologically classified as Mils. This divergence in morphological classification and transcriptomic profile suggests that these oocytes are in a low activity state, possibly as a transitional phase before maturation or a holding state. To assess for confounding variables in our transcriptomic analysis, we assessed expression of cell cycle, apoptosis and oxidative stress genes and did not detect any significant patterns, indicating that the oocytes were not biased or stressed (FIG. 16). Altogether this observation suggests that MII oocytes derived from OSC-IVM were transcriptionally more similar to those from the IVF-MII condition compared to Media-IVM control.

Finally, to determine the ratio of MII oocytes with a strong IVF MII maturation signature in each experimental condition, we calculated the percentage of cells in clusters 0 and 2, identified as containing oocytes with a ‘IVF-like MII’ signature (FIG. 17D). As expected, the strong majority (91%) of MII oocytes from the IVF-MII condition were classified within clusters 0 and 2. As a continued indication of positive maturation impact, co-culture with OSCs led to generation of 79% MII oocytes with an ‘IVF-like MII’ profile (cluster 0 and 2). In comparison, just 56% of resultant MII oocytes from the Media-IVM condition were found in ‘IVF-like MII’ profile clusters. This population distribution is significantly different from random (X² test, α=0.00632). Altogether, we conclude that OSC-IVM supports formation of MII oocytes with high transcriptomic similarity to IVF matured MII oocytes and highlights the potential of using this novel approach to rescue denuded immature oocytes from IVF procedures.

### Example 13. Granulosa-Like Cells Support Germ Cell Development within Ovaroids

Current methods for inducing and culturing human primordial germ cell-like cells (hPGCLCs) produce cells corresponding to immature, premigratory primordial germ cells (PGCs) that lack expression of gonadal PGC markers such as DAZL. During fetal development, PGCs mature through interactions with gonadal somatic cells, with DAZL playing a key role in downregulation of pluripotency factors and commitment to gametogenesis. This process has recently been recreated in vitro using mouse fetal ovarian somatic cells, which allowed the development of hPGCLCs to the oogonia-like stage. We hypothesized that in vitro-derived human granulosa-like cells could perform a similar role, with the potential for eliminating interspecies developmental mismatches. Therefore, we combined our granulosa-like cells with hPGCLCs to form ovarian organoids, which we termed ovaroids.

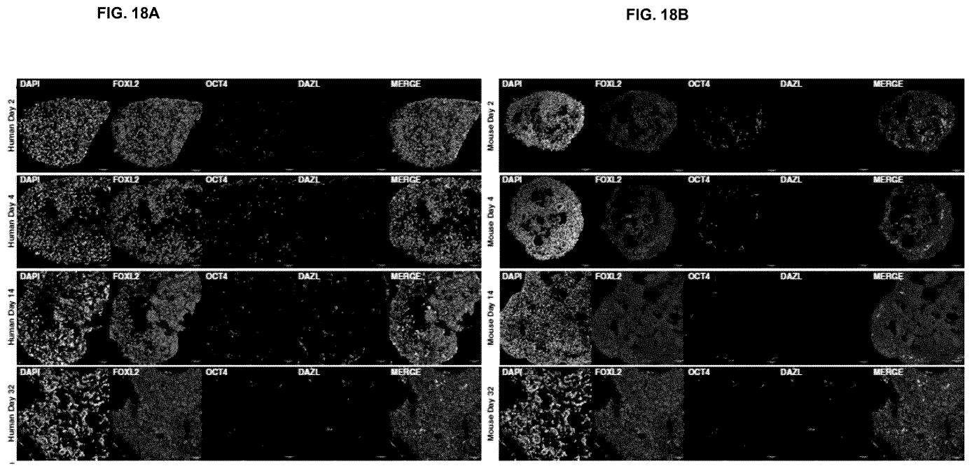

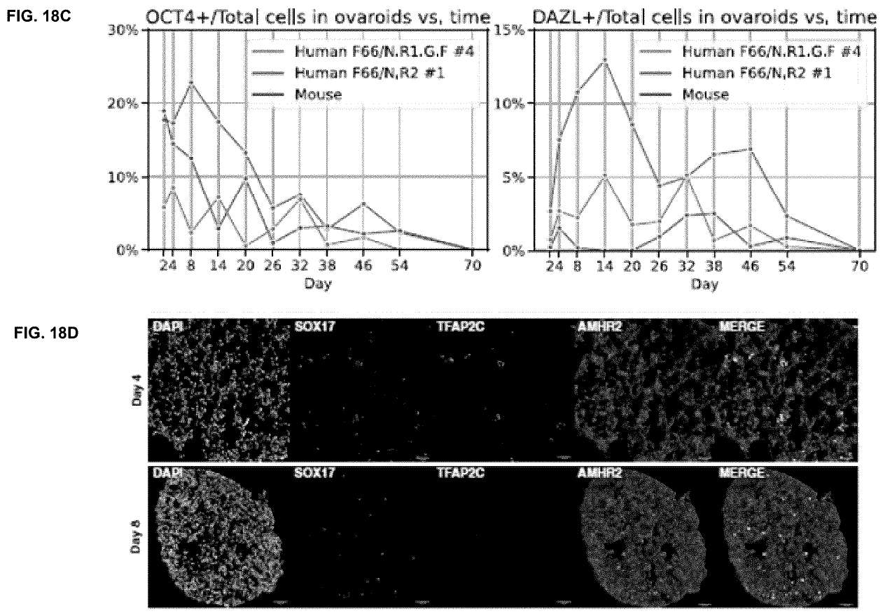

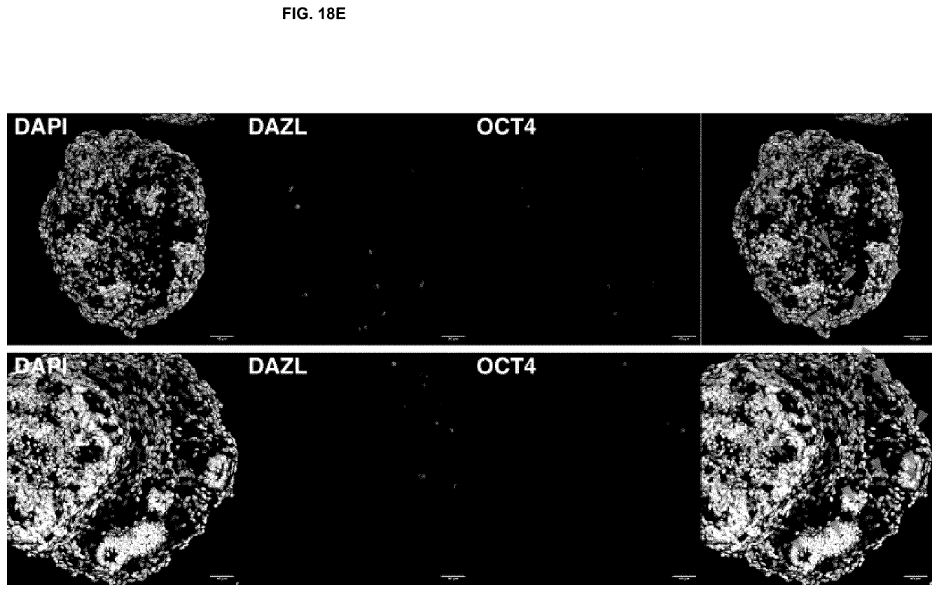

To generate ovaroids, we aggregated these two cell types in low-binding U-bottom wells, followed by transfer to air-liquid interface Transwell culture. As a comparison, we followed a previously described protocol to isolate fetal mouse ovarian somatic cells and aggregate them with hPGCLCs. By immunofluorescence, we observed expression of the mature marker DAZL beginning in a subset of OCT4+hPGCLCs at 4 days of co-culture with hiPSC-derived granulosa-like cells (FIG. 18A). In contrast, robust DAZL expression in co-culture with mouse cells was not observed until day 32 (FIG. 18B), with fainter expression visible at day 26. Similarly, in a previous study using the same hPGCLC line and anti-DAZL antibody, DAZL expression was observed only after 77 days of co-culture with mouse fetal testis somatic cells.

The fraction of DAZL+ cells reached its maximum at day 14 in human ovaroids and day 38 in mouse ovaroids (FIG. 18C). In human ovaroids, the fraction of OCT4+ cells declined after day 8. In mouse ovaroids, the fraction of OCT4+ cells also declined over time. At day 16 in human ovaroids, DAZL+OCT4− cells were also apparent (FIG. 18E) in addition to DAZL+OCT4+ cells, and past day 38 there were more DAZL+ cells than OCT4+ cells in total (FIG. 18C). The downregulation of OCT4 in DAZL+ oogonia occurs in vivo during the second trimester of human fetal ovarian development; however, we did not observe the transition of DAZL to exclusively cytoplasmic localization that was reported to take place at this stage. Expression of TFAP2C, an early-stage PGC marker, declined during ovaroid culture (FIG. 18D) and was almost entirely absent by day 8. By contrast, SOX17 expression was still visible on day 8, and OCT4 and DAZL expression continued to day 54 (FIG. 18A, C).

Although this system allowed the rapid development of hPGCLCs to the gonadal stage, the number of germ cells in both hiPSC- and mouse-derived ovaroids declined over prolonged culture (FIG. 18C), indicating that either they were dying or differentiating to other lineages. Unlike mouse-derived ovaroids, the hiPSC-derived ovaroids cultured on Transwells gradually flattened and widened, and by day 38 were largely collapsed.

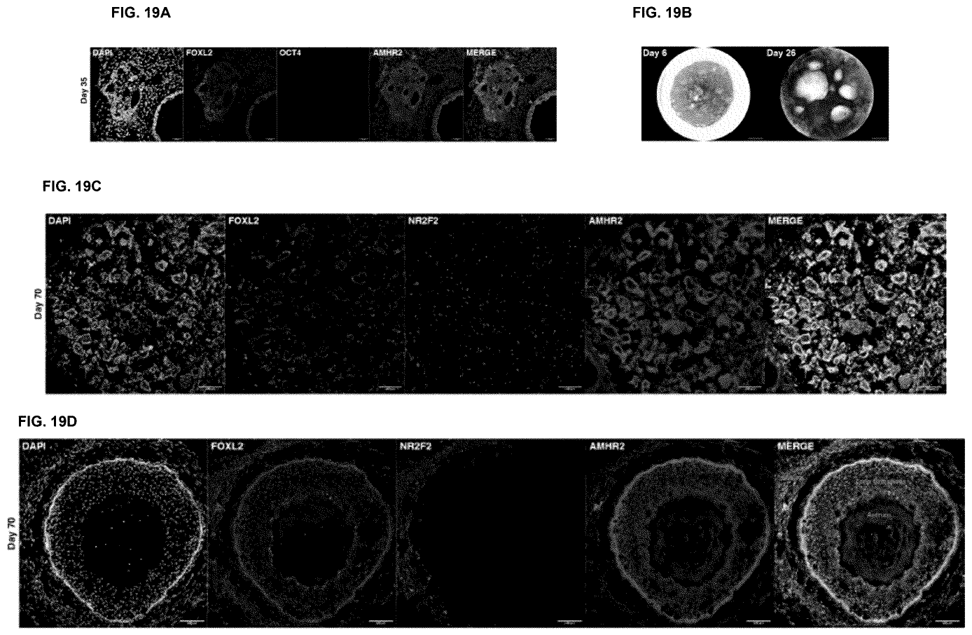

Nonetheless, in these longer-term experiments, we observed the formation of empty follicle-like structures composed of cuboidal AMHR2+FOXL2+ granulosa-like cells (FIG. 19A), suggesting that the TFs could drive folliculogenesis even in the absence of oocytes. Follicle-like structure formation was first visible at day 16 (FIG. 18E), and at day 26 the largest of these structures had grown to 1-2 mm diameter (FIG. 19B). At day 70, ovaroids had developed follicles of a variety of sizes, mainly small single-layer follicles (FIG. 19C) but also including antral follicles (FIG. 19D). Cells outside of the follicles stained positive for NR2F2 (FIG. 19C, D), a marker of ovarian stromal and theca cells.

To further examine the gene expression of hPGCLCs and somatic cells in this system, we performed scRNA-seq on dissociated ovaroids at days 2, 4, 8, and 14 of culture, and clustered cells according to gene expression. As expected, the largest cluster (cluster 0) contained cells expressing granulosa markers such as FOXL2, WNT4, and CD82 (FIG. 19A, B). Cells expressing markers of secondary/antral granulosa cells such as FSHR and CYP19A1 were also found within this cluster, although these were much less numerous. A smaller cluster (cluster 1) expressing the ovarian stromal marker NR2F2 was also present. NR2F2 is expressed by both stromal and theca cells, but the cells in cluster 1 did not express 17a-hydroxylase (CYP17A1), indicating that they could not produce androgens and were not theca cells.

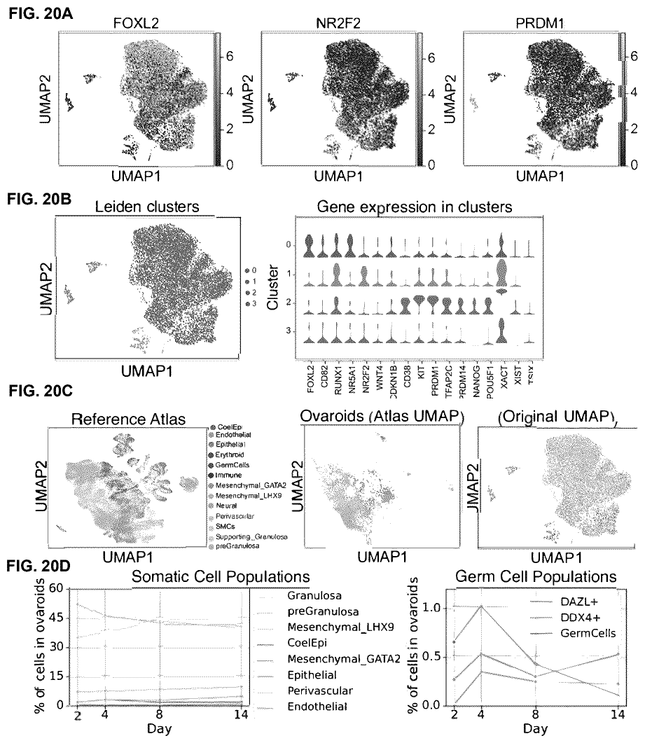

We also observed a cluster of hPGCLCs expressing marker genes such as CD38, KIT, PRDM1, TFAP2C, PRDM14, NANOG, and POU5F1. Notably, X-chromosomal lncRNAs XIST, TSIX, and XACT were all more highly expressed (an average of ˜80-, ˜20-, and ˜2900-fold, respectively) in the hPGCLCs relative to other clusters (FIG. 20B), suggesting that the hPGCLCs were starting the process of X-reactivation, which in hPGCs is associated with high expression of both XIST and XACT. The X-chromosomal HPRT1 gene, known to be more highly expressed in cells with two active X chromosomes, was also ˜3-fold upregulated.

We next compared our in vitro-generated ovaroids to a reference atlas of human fetal ovarian development. We used scanpy ingest to integrate our samples into the atlas and annotate each cell with the closest cell type from the in vivo data (FIG. 20C). The ovaroids consisted mainly of granulosa, gonadal mesenchyme, and pre-granulosa lineages (FIG. 20D), with a small fraction of coelomic epithelium. The fraction of granulosa cells increased from day 2 through day 8, potentially representing a maturation of the somatic cell population. As expected, neural, immune, smooth muscle, and erythroid cells, which were present in fetal ovaries, were completely absent from our ovaroids. Epithelial, endothelial, and perivascular cells were detected, but at very low frequency (1% or less), possibly representing a low rate of off-target differentiation.

We additionally examined the overall fraction of germ cells, as well as the fraction of cells expressing the gonadal germ cell markers DAZL and DDX4, over the course of our experiment (FIG. 20D). We defined the germ cell population based on the fetal ovary atlas integration. This population increased from days 2 to 4 but declined thereafter. In comparison, the fraction of DAZL+ and DDX4+ cells also increased from days 2 to 4 but remained roughly constant from days 4 to 14 (FIG. 20D). We performed a differential gene expression analysis and gene ontology enrichment on DAZL+ cells relative to DAZL− cells. Upregulated genes (log 2fc>2, n=221) were most highly enriched for terms related to generic developmental processes but also included terms related to adhesion and migration (e.g., ‘ameboidal-type cell migration’), as well as reproductive system development. Downregulated genes (log 2fc<−2, n=6451) were strongly related to metabolic processes and mitotic cell. These data suggest that DAZL+ cells in our ovaroids are downregulating their metabolism and proliferation, in agreement with the known role of DAZL in suppressing PGC proliferation.

### Example 14. Pre-Clinical Trials

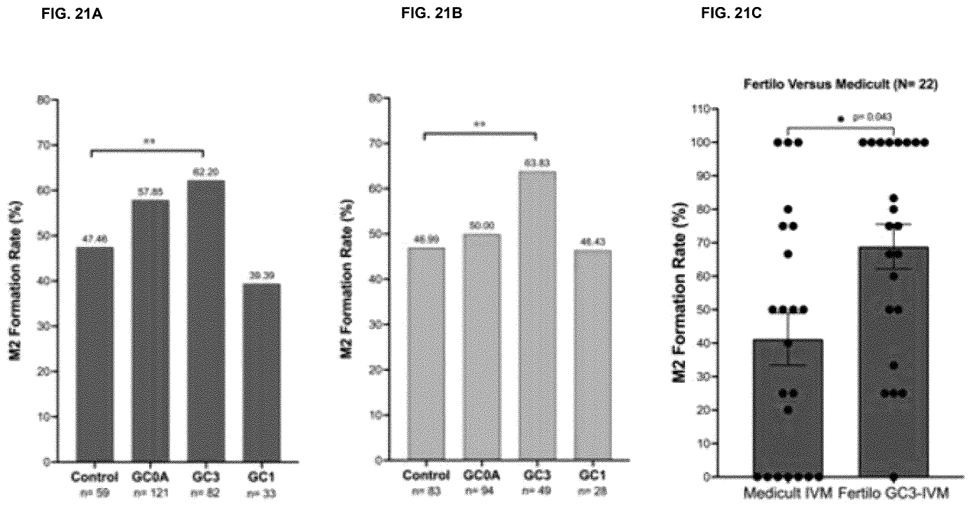

Preclinical trials of the OSCs-IVM system were performed using cell culture media-matched controls in a sibling oocyte study for both human denuded immature oocytes retrieved after standard of care gonadotropin stimulation, and intact immature COCs retrieved after minimal gonadotropin stimulation. The control condition contained an identical media formulation as the OSCs-IVM condition, with the only difference between conditions being the presence of the OSCs in the OSC-IVM. Results show that the OSCs-IVM system statistically significantly improved oocyte maturation rate, determined by the presence of a polar body, by ˜15% with denuded oocytes from standard of care (FIG. 21A) and by ˜17% in intact COCs from minimal stimulation (FIG. 21B). OSCs-IVM were compared to the clinically approved Medicult-IVM system, which is marketed for use with intact COCs after minimal stimulation. OSC-IVM statistically significantly improves oocyte maturation rates by ˜28% on average per study donor, compared to Medicult-IVM in an on-label, sibling oocyte study (FIG. 21C).

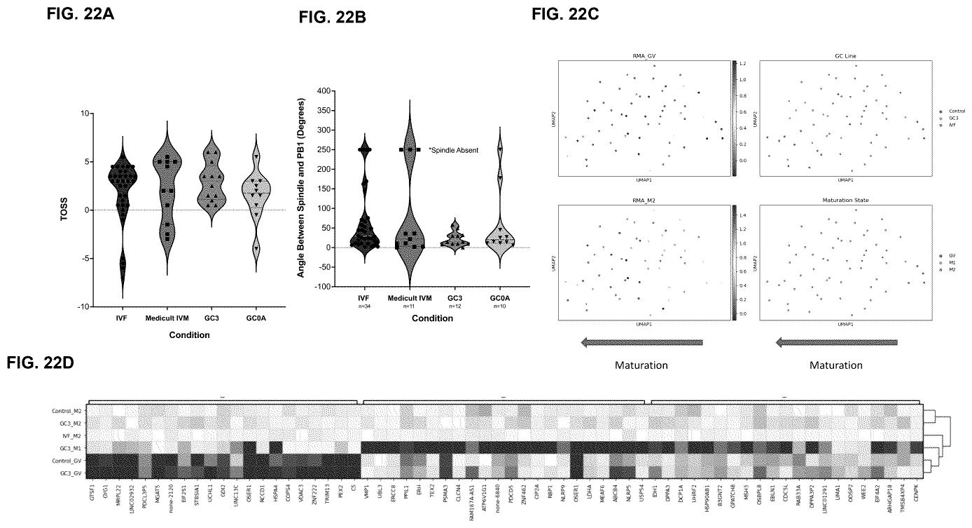

It was also determined if OSC co-culture could improve oocyte quality. While no universally accepted method exists yet to determine “oocyte quality”, studies have shown that certain morphological and molecular features can be used to infer oocyte quality, as these features are correlated with improvements in embryo formation and live birth rates in IVF. One such measure is a total oocyte score (TOS) generated from manual qualitative assessment of six morphological features of mature oocytes: oocyte size, zona size, color/shape, cytoplasmic granularity, polar body quality, and PVS quality. Another metric of quality is spindle assembly position, which has been shown as a reliable metric of oocyte quality by measuring the angle between polar body 1 (PB1) and the spindle apparatus, with a decrease in angle correlated with an improvement in oocyte quality. Lastly, certain genetic markers identified in transcriptomic analysis have been correlated with oocyte quality, measuring indications such as oxidative stress, embryogenesis competence, and DNA damage. All three of these metrics were employed here to determine if OSCs-IVM could improve oocyte quality relative to media matched controls. Using a limited set of denuded immature oocytes and IVF in vivo MII controls, it was determined that the OSCs described herein trend towards improvement of oocyte quality compared to media-matched controls and show similarity with in vivo MII oocytes in terms of morphological quality (FIG. 22A). OSCs-IVM were likewise shown to on average decrease the angle between the PB1 and spindle compared to media-matched controls and IVF in vivo Mils, with no instance of spindle absence in OSCs-IVM Mils (FIG. 22B). Additionally, through differential gene expression analysis (DGEA), it was evidenced that the OSCs-IVM oocytes show high similarity to in vivo MII oocytes, with expected expression of key embryogenesis competence genes (FIG. 22C-3D).

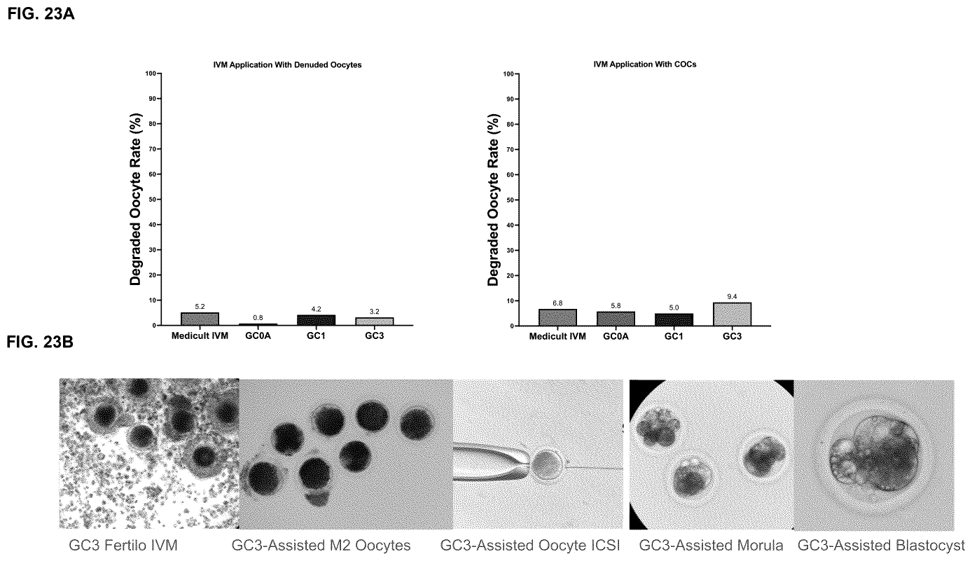

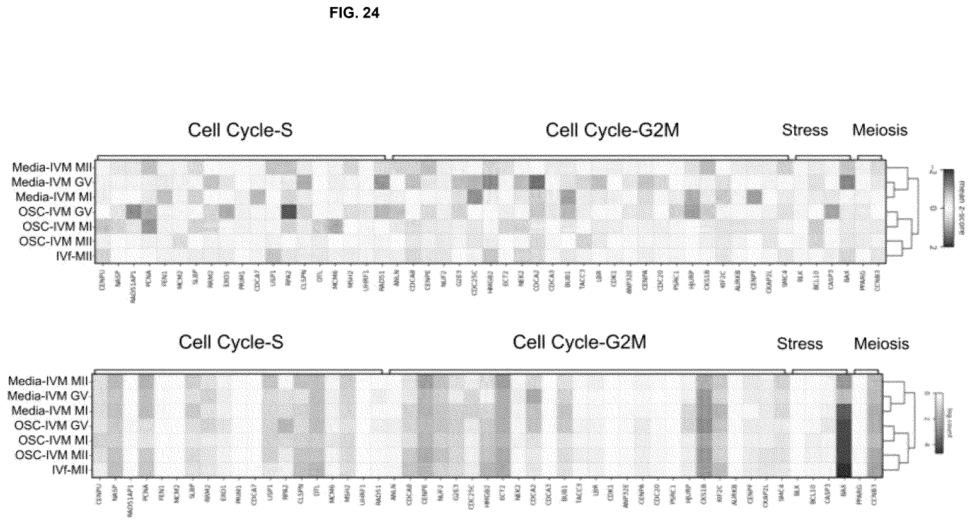

Additionally, both human and porcine animal models were studied to determine toxicity of the OSCs co-culture. Utilizing human preclinical models, the OSCs-IVM condition was performed and assessed for oocyte outcomes considered as “degraded”, meaning the oocytes are undergoing a rapid state of apoptosis or cell death. The OSCs-IVM results in no significant enhancement in oocyte degradation rate in human oocytes compared to the Medicult-IVM media alone (FIG. 23A). We likewise assessed whether porcine oocytes matured in the presence of the OSC-IVM product were capable of forming blastocysts. As can be seen, we were able to successfully fertilize and generate blastocysts in the OSC-IVM condition for porcine oocytes (FIG. 23B). While these porcine studies are not designed to test efficacy, they demonstrate that OSCs-IVM is not toxic to oocytes or preventative of embryo formation.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, apparatuses, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

### Specific Embodiments

Several non-limiting, exemplary embodiments of the disclosure are enumerated below. The below embodiments should not be construed to limit the scope of the invention, rather, the below are presented as some examples of the utility of the invention.

1. A method of preparing one or more oocytes that have previously been retrieved from a human subject for use in an assisted reproduction technology (ART) procedure, the method comprising co-culturing the one or more oocytes with a population of ovarian support cells.

2. A method of producing a mature oocyte for use in an ART procedure, the method comprising co-culturing one or more oocytes that have previously been retrieved from a human subject with a population of ovarian support cells.

3. A method of inducing oocyte maturation in vitro, the method comprising co-culturing one or more oocytes that have previously been retrieved from a human subject with a population of ovarian support cells, wherein the co-culturing is conducted for a period of from about 6 hours to about 120 hours.

4. The method of any one of embodiments 1-3, wherein the subject is not administered a follicular triggering agent prior to retrieval of the one or more oocytes from the subject.

5. The method of any one of embodiments 1-3, wherein prior to retrieval of the one or more oocytes from the subject, the subject is administered one or more follicular triggering agents during a follicular triggering period.

6. The method of embodiment 5, wherein the follicular triggering period has a duration of no greater than 8 days.

7. The method of embodiment 6, wherein the follicular triggering period has a duration of no greater than 7 days.

8. The method of embodiment 7, wherein the follicular triggering period has a duration of no greater than 6 days.

9. The method of embodiment 8, wherein the follicular triggering period has a duration of no greater than 5 days.

10. The method of embodiment 9, wherein the follicular triggering period has a duration of no greater than 4 days.

11. The method of embodiment 10, wherein the follicular triggering period has a duration of no greater than 3 days.

12. The method of embodiment 11, wherein the follicular triggering period has a duration of no greater than 2 days.

13. The method of embodiment 12, wherein the follicular triggering period has a duration of no greater than 1 day.

14. The method of embodiment 5, wherein the follicular triggering period has a duration of from 1 day to 8 days.

15. The method of embodiment 14, wherein the follicular triggering period has a duration of from 1 day to 7 days.

16. The method of embodiment 15, wherein the follicular triggering period has a duration of from 1 day to 6 days.

17. The method of embodiment 16, wherein the follicular triggering period has a duration of from 1 day to 5 days.

18. The method of embodiment 17, wherein the follicular triggering period has a duration of from 1 day to 4 days.

19. The method of embodiment 18, wherein the follicular triggering period has a duration of from 1 day to 3 days.

20. The method of embodiment 5, wherein the follicular triggering period has a duration of from 2 days to 8 days.

21. The method of embodiment 20, wherein the follicular triggering period has a duration of from 2 days to 7 days.

22. The method of embodiment 21, wherein the follicular triggering period has a duration of from 2 days to 6 days.

23. The method of embodiment 22, wherein the follicular triggering period has a duration of from 2 days to 5 days.

24. The method of embodiment 23, wherein the follicular triggering period has a duration of from 2 days to 4 days.

25. The method of embodiment 5, wherein the follicular triggering period has a duration of from 3 days to 8 days.

26. The method of embodiment 25, wherein the follicular triggering period has a duration of from 3 days to 7 days.

27. The method of embodiment 26, wherein the follicular triggering period has a duration of from 3 days to 6 days.

28. The method of embodiment 27, wherein the follicular triggering period has a duration of from 3 days to 5 days.

29. The method of any one of embodiments 5-28, wherein the one or more follicular triggering agents comprise follicle stimulating hormone (FSH), clomiphene citrate, and/or human chorionic gonadotropin (hCG).

30. The method of embodiment 29, wherein the one or more follicular triggering agents comprise FSH.

31. The method of embodiment 30, wherein the FSH is administered to the subject in one or more doses per day.

32. The method of embodiment 31, wherein the FSH is administered to the subject once daily.

33. The method of any one of embodiments 30-32, wherein the FSH is administered to the subject in an amount of from about 100 international units (IU) to about 1,000 IU per day.

34. The method of embodiment 33, wherein the FSH is administered to the subject in an amount of from about 200 IU to about 800 IU per day.

35. The method of embodiment 34, wherein the FSH is administered to the subject in an amount of from about 300 IU to about 700 IU per day.

36. The method of embodiment 35, wherein the FSH is administered to the subject in an amount of from about 300 IU to about 600 IU per day, from about 300 IU to about 500 IU per day, or from about 300 IU to about 400 IU per day.

37. The method of any one of embodiments 30-36, wherein the duration of FSH administration is equal to the duration of the follicular triggering period.

38. The method of any one of embodiments 30-36, wherein the duration of FSH administration is less than the duration of the follicular triggering period.

39. The method of embodiment 38, wherein the duration of FSH administration is 1, 2, 3, 4, or 5 days during the follicular triggering period, optionally wherein the FSH is administered to the subject in an amount of about 200 IU per day for 1, 2, 3, 4, or 5 days during the follicular triggering period, optionally wherein the FSH is administered to the subject in an amount of about 200 IU per day for 3 days during the follicular triggering period.

40. The method of any one of embodiments 29-39, wherein the one or more follicular triggering agents comprise clomiphene citrate.

41. The method of embodiment 40, wherein the clomiphene citrate is administered to the subject in one or more doses per day.

42. The method of embodiment 41, wherein the clomiphene citrate is administered to the subject once daily.

43. The method of any one of embodiments 40-42, wherein the clomiphene citrate is administered to the subject in an amount of from about 50 mg to about 100 mg per day.

44. The method of embodiment 43, wherein the clomiphene citrate is administered to the subject in an amount of about 50 mg per day.

45. The method of any one of embodiments 40-44, wherein the duration of clomiphene citrate administration is equal to the duration of the follicular triggering period.

46. The method of any one of embodiments 40-44, wherein the duration of clomiphene citrate administration is less than the duration of the follicular triggering period.

47. The method of embodiment 46, wherein the duration of clomiphene citrate administration is 1, 2, 3, 4, or 5 days during the follicular triggering period.

48. The method of any one of embodiments 29-47, wherein the one or more follicular triggering agents comprise hCG.

49. The method of embodiment 48, wherein the hCG is administered to the subject in one or more doses per day.

50. The method of embodiment 49, wherein the hCG is administered to the subject in 1, 2, or 3 doses during the follicular triggering period.

51. The method of any one of embodiments 48-50, wherein the hCG is administered to the subject in an amount of from about 200 μg to about 700 μg per dose.

52. The method of embodiment 51, wherein the hCG is administered to the subject in an amount of from about 200 μg to about 500 μg per dose, from about 300 μg to about 600 μg per dose, from about 400 μg to about 700 μg per dose, from about 200 μg to about 300 μg per dose, from about 300 μg to about 400 μg per dose, from about 400 μg to about 500 μg per dose, from about 500 μg to about 600 μg per dose, or from about 600 μg to about 700 μg per dose.

53. The method of embodiment 52, wherein the hCG is administered to the subject in an amount of about 500 μg per dose.

54. The method of any one of embodiments 48-50, wherein the hCG is administered to the subject in an amount of from about 2,500 IU to about 10,000 IU per dose.

55. The method of any one of embodiments 5-54, wherein the subject is one that has completed oral contraceptive treatment within 28 days of commencement of the follicular triggering period.

56. The method of embodiment 55, wherein the follicular triggering period commences at least 5 days after cessation of the contraceptive treatment.

57. The method of any one of embodiments 5-54, wherein the subject has not undergone oral contraceptive treatment within 28 days of commencement of the follicular triggering period.

58. The method of embodiment 57, wherein the follicular triggering period commences on day 2 of the subject's menstrual cycle.

59. The method of any one of embodiments 55-58, wherein the contraceptive treatment comprises administration to the subject of a gonadotropin-releasing hormone (GnRH) agonist.

60. The method of any one of embodiments 5-59, wherein the subject has been determined to exhibit a follicle size of from about 6 mm to about 8 mm prior to commencement of the follicular triggering period.

61. The method of any one of embodiments 5-59, wherein the subject has been determined to exhibit a follicle size of from about 6 mm to about 8 mm prior to administration of a final follicular triggering agent.

62. The method of any one of embodiments 1-61, wherein a biological sample isolated from the subject prior to retrieval of the one or more oocytes has been determined to have an anti-Mullerian hormone (AMH) concentration of from about 0.1 ng/mL to about 1 ng/mL, or from about 1 ng/mL to about 6 ng/mL.

63. The method of embodiment 62, wherein the biological sample has been determined to have an AMH concentration of from about 1 ng/mL to about 6 ng/mL, optionally wherein the biological sample has been determined to have an AMH concentration of from about 2.5 ng/mL to about 3.0 ng/mL.

64. The method of any one of embodiments 1-61, wherein a biological sample isolated from the subject prior to retrieval of the one or more oocytes has been determined to have an AMH concentration of at least 1 ng/mL.

65. The method of any one of embodiments 1-61, wherein a biological sample isolated from the subject prior to retrieval of the one or more oocytes has been determined to have an AMH concentration of no greater than 6 ng/mL.

66. The method of any one of embodiments 1-61, wherein a biological sample isolated from the subject prior to retrieval of the one or more oocytes has been determined to have an AMH concentration of from about 0.1 ng/mL to about 1 ng/mL.

67. The method of any one of embodiments 62-66, wherein the biological sample is a blood sample.

68. The method of any one of embodiments 1-67, wherein the subject is from 18 years old to 48 years old at the time of retrieval of the one or more oocytes.

69. The method of embodiment 68, wherein the subject is from 20 years old to 45 years old at the time of retrieval of the one or more oocytes.

70. The method of embodiment 68, wherein the subject is less than 35 years old at the time of retrieval of the one or more oocytes.

71. The method of embodiment 68, wherein the subject is greater than 35 years old at the time of retrieval of the one or more oocytes.

72. The method of any one of embodiments 1-71, wherein prior to retrieval of the one or more oocytes from the subject, the subject has been determined to exhibit a follicle size of from about 6 mm to about 14 mm.

73. The method of embodiment 72, wherein prior to retrieval of the one or more oocytes from the subject, the subject has been determined to exhibit a follicle size of from about 8 mm to about 12 mm.

74. The method of embodiment 73, wherein prior to retrieval of the one or more oocytes from the subject, the subject has been determined to exhibit a follicle size of from about 8 mm to about 9 mm.

75. The method of any one of embodiments 1-71, wherein prior to retrieval of the one or more oocytes from the subject, the subject has been determined to exhibit a follicle size of no greater than 14 mm.

76. The method of any one of embodiments 72-75, wherein the follicle size has been assessed by way of ultrasound image analysis.

77. The method of any one of embodiments 1-76, wherein a total of 20 oocytes or less are retrieved from the subject.

78. The method of embodiment 77, wherein 15 oocytes or less are retrieved from the subject.

79. The method of embodiment 78, wherein 10 oocytes or less are retrieved from the subject.

80. The method of embodiment 79, wherein 9 oocytes or less are retrieved from the subject.

81. The method of embodiment 80, wherein 8 oocytes or less are retrieved from the subject.

82. The method of embodiment 81, wherein 7 oocytes or less are retrieved from the subject.

83. The method of embodiment 82, wherein 6 oocytes or less are retrieved from the subject.

84. The method of embodiment 83, wherein 5 oocytes or less are retrieved from the subject.

85. The method of any one of embodiments 1-84, wherein a plurality of oocytes are retrieved from the subject.

86. The method of embodiment 85, wherein from 10% to 100% of the oocytes retrieved from the subject are germinal vesicle (GV)-stage or meiosis I (MI)-stage oocytes.

87. The method of embodiment 86, wherein from 20% to 100% of the oocytes retrieved from the subject are GV-stage or MI-stage oocytes.

88. The method of embodiment 87, wherein from 30% to 100% of the oocytes retrieved from the subject are GV-stage or MI-stage oocytes.

89. The method of embodiment 88, wherein from 40% to 100% of the oocytes retrieved from the subject are GV-stage or MI-stage oocytes.

90. The method of embodiment 89, wherein from 50% to 100% of the oocytes retrieved from the subject are GV-stage or MI-stage oocytes.

91. The method of embodiment 90, wherein from 60% to 100% of the oocytes retrieved from the subject are GV-stage or MI-stage oocytes.

92. The method of embodiment 91, wherein from 70% to 100% of the oocytes retrieved from the subject are GV-stage or MI-stage oocytes.

93. The method of embodiment 92, wherein from 80% to 100% of the oocytes retrieved from the subject are GV-stage or MI-stage oocytes.

94. The method of embodiment 93, wherein from 90% to 100% of the oocytes retrieved from the subject are GV-stage or MI-stage oocytes.

95. The method of embodiment 94, wherein 100% of the oocytes retrieved from the subject are GV-stage or MI-stage oocytes.

96. The method of any one of embodiments 1-95, wherein the population of ovarian support cells comprises ovarian granulosa cells and/or ovarian stroma cells, optionally wherein the ovarian granulosa cells are forkhead box protein L2 (FOXL2)-positive and/or wherein the ovarian stroma cells are nuclear receptor subfamily 2 group F member 2 (NR2F2)-positive.

97. The method of any one of embodiments 1-96, wherein the population of ovarian support cells comprises from about 50,000 to about 500,000 ovarian support cells.

98. The method of any one of embodiments 1-97, wherein the population of ovarian support cells comprises from about 50,000 to about 60,000 ovarian support cells, from about 60,000 to about 70,000 ovarian support cells, from about 70,000 to about 80,000 ovarian support cells, from about 80,000 to about 90,000 ovarian support cells, from about 90,000 to about 100,000 ovarian support cells, or from about 100,000 to about 150,000 ovarian support cells, optionally wherein the population of ovarian support cells comprises about 125,000 ovarian support cells.

99. The method of any one of embodiments 1-98, wherein the population of ovarian support cells comprises about 50,000 ovarian support cells, about 55,000 ovarian support cells, about 60,000 ovarian support cells, about 65,000 ovarian support cells, about 70,000 ovarian support cells, about 75,000 ovarian support cells, about 80,000 ovarian support cells, about 85,000 ovarian support cells, about 90,000 ovarian support cells, about 95,000 ovarian support cells, about 100,000 ovarian support cells, about 105,000 ovarian support cells, about 110,000 ovarian support cells, about 115,000 ovarian support cells, about 120,000 ovarian support cells, about 125,000 ovarian support cells, about 130,000 ovarian support cells, about 135,000 ovarian support cells, about 140,000 ovarian support cells, about 145,000 ovarian support cells, or about 150,000 ovarian support cells.

100. The method of any one of embodiments 96-99, wherein the ovarian support cells comprise steroidogenic granulosa cells.

101. The method of embodiment 100, wherein the steroidogenic granulosa cells produce estradiol.

102. The method of any one of embodiments 1-101, wherein the ovarian support cells are obtained by differentiation of a population of induced pluripotent stem cells (iPSCs).

103. The method of embodiment 102, wherein the ovarian support cells are obtained by modifying the iPSCs to express one or more transcription factors selected from FOXL2, NR5A1, GATA4, RUNX1, and RUNX2.

104. The method of embodiment 103, wherein the ovarian support cells are obtained by modifying the iPSCs to express two or more of FOXL2, NR5A1, GATA4, RUNX1, and RUNX2.

105. The method of embodiment 104, wherein the ovarian support cells are obtained by modifying the iPSCs to express three or more of FOXL2, NR5A1, GATA4, RUNX1, and RUNX2.

106. The method of embodiment 105, wherein the ovarian support cells are obtained by modifying the iPSCs to express four or more of FOXL2, NR5A1, GATA4, RUNX1, and RUNX2.

107. The method of embodiment 106, wherein the ovarian support cells are obtained by modifying the iPSCs to express all five of FOXL2, NR5A1, GATA4, RUNX1, and RUNX2.

108. The method of any one of embodiments 1-107, wherein the ovarian support cells are cryopreserved and thawed prior to the co-culturing with the one or more oocytes.

109. The method of embodiment 108, wherein the ovarian support cells are thawed from about 24 hours to about 120 hours prior to the co-culturing with the one or more oocytes.

110. The method of embodiment 108, wherein the ovarian support cells are thawed from about 24 hours to about 48 hours, from about 48 hours to about 72 hours, from about 72 hours to about 96 hours, or from about 96 hours to about 120 hours prior to the co-culturing with the one or more oocytes.

111. The method of embodiment 108, wherein the ovarian support cells are thawed from about 24 hours to about 36 hours, from about 30 hours to about 40 hours, from about 36 hours to about 48 hours, from about 48 hours to about 56 hours, from about 56 hours to about 72 hours, from about 72 hours to about 84 hours, from about 80 hours to about 96 hours, from about 90 hours to about 100 hours, from about 96 hours to about 108 hours, or from about 108 hours to about 120 hours prior to the co-culturing with the one or more oocytes.

112. The method of any one of embodiments 1-111, wherein the one or more oocytes are co-cultured with the population of ovarian support cells for from about 12 hours to about 120 hours.

113. The method of any one of embodiments 1-111, wherein the one or more oocytes are co-cultured with the population of ovarian support cells for from about 12 hours to about 24 hours, from about 12 hours to about 36 hours, from about 24 hours to about 48 hours, from about 36 hours to about 60 hours, from about 54 hours to about 72 hours, from about 68 hours to about 96 hours, or from about 96 hours to about 120 hours.

114. The method of any one of embodiments 1-111, wherein the one or more oocytes are co-cultured with the population of ovarian support cells for about 12 hours, about 14 hours, about 16 hours, about 18 hours, about 20 hours, about 22 hours, about 24 hours, about 26 hours, about 28 hours, about 30 hours, about 32 hours, about 34 hours, about 36 hours, about 38 hours, about 40 hours, about 42 hours, about 44 hours, about 46 hours, about 48 hours, about 50 hours, about 52 hours, about 54 hours, about 56 hours, about 58 hours, about 60 hours, about 62 hours, about 64 hours, about 66 hours, about 68 hours, about 70 hours, about 72 hours, about 74 hours, about 76 hours, about 78 hours, about 80 hours, about 82 hours, about 84 hours, about 86 hours, about 88 hours, about 90 hours, about 92 hours, about 94 hours, about 96 hours, about 98 hours, about 100 hours, about 102 hours, about 104 hours, about 106 hours, about 108 hours, about 110 hours, about 112 hours, about 114 hours, about 116 hours, about 118 hours, or about 120 hours.

115. The method of any one of embodiments 1-114, wherein the co-culturing is conducted in an adherent co-culture system.

116. The method of any one of embodiments 1-114, wherein the co-culturing is conducted in a suspension co-culture system.

117. The method of any one of embodiments 1-116, wherein prior to and/or after the co-culturing, the one or more oocytes are evaluated for a parameter selected from the group consisting of total oocyte score, GV-stage to MII-stage oocyte maturation rate, GV-stage to MI-stage oocyte maturation rate, MI-stage to MII-stage oocyte maturation rate, average oocyte shape, average oocyte size, average ooplasm quality, average perivitelline space (PVS) quality, average zona pellucida (ZP) quality, and average polar body quality.

118. The method of any one of embodiments 1-117, wherein the one or more oocytes are denuded following the co-culturing.

119. The method of any one of embodiments 1-118, the method further comprising isolating one or more meiosis II (MII)-stage oocytes from the mixture produced by co-culturing the one or more oocytes retrieved from the subject with the population of ovarian support cells.

120. The method of embodiment 119, wherein the subject is undergoing an autologous ART procedure, and wherein the method further comprises contacting each of the one or more MII-stage oocytes with a mature sperm cell.

121. The method of embodiment 120, wherein the one or more MII-stage oocytes are cryopreserved and thawed prior to the contacting.

122. The method of embodiment 120, wherein the one or more MII-stage oocytes are not cryopreserved and thawed prior to the contacting.

123. The method of any one of embodiments 120-122, wherein the contacting comprises in vitro fertilization (IVF) of the one or more MII-stage oocytes.

124. The method of any one of embodiments 120-122, wherein the contacting comprises intracytoplasmic sperm injection (ICSI) into the one or more MII-stage oocytes.

125. The method of any one of embodiments 120-124, wherein the contacting results in formation of an embryo.

126. The method of embodiment 125, wherein the embryo is transferred to the uterus of the subject.

127. The method of embodiment 126, wherein the embryo is transferred to the uterus of the subject about 3 days following the contacting of the one or more MII-stage oocytes with a mature sperm cell.

128. The method of embodiment 126, wherein the embryo is transferred to the uterus of the subject about 5 days following the contacting of the one or more MII-stage oocytes with a mature sperm cell.

129. The method of embodiment 126, wherein the embryo transferred to the uterus of the subject is a blastocyst-stage embryo.

130. A method of producing a mature oocyte for use in an ART procedure, the method comprising:
(a) administering to a human subject one or more follicular triggering agents during a follicular triggering period;
(b) retrieving one or more oocytes from the subject following the follicular triggering period; and
(c) culturing the one or more oocytes with a population of ovarian support cells, thereby producing one or more mature oocytes.

131. A method of promoting oocyte maturation for a subject undergoing an ART procedure and that has previously been administered one or more follicular triggering agents during a follicular triggering period, the method comprising:
(a) retrieving one or more oocytes from the subject;
(b) culturing the one or more oocytes with a population of ovarian support cells, thereby producing one or more mature oocytes; and
(c) isolating the one or more mature oocytes.

132. The method of embodiment 130 or 131, wherein the follicular triggering period has a duration of no greater than 8 days.

133. The method of embodiment 132, wherein the follicular triggering period has a duration of no greater than 7 days.

134. The method of embodiment 133, wherein the follicular triggering period has a duration of no greater than 6 days.

135. The method of embodiment 134, wherein the follicular triggering period has a duration of no greater than 5 days.

136. The method of embodiment 135, wherein the follicular triggering period has a duration of no greater than 4 days.

137. The method of embodiment 136, wherein the follicular triggering period has a duration of no greater than 3 days.

138. The method of embodiment 137, wherein the follicular triggering period has a duration of no greater than 2 days.

139. The method of embodiment 138, wherein the follicular triggering period has a duration of no greater than 1 day.

140. The method of embodiment 130 or 131, wherein the follicular triggering period has a duration of from 1 day to 8 days.

141. The method of embodiment 140, wherein the follicular triggering period has a duration of from 1 day to 7 days.

142. The method of embodiment 141, wherein the follicular triggering period has a duration of from 1 day to 6 days.

143. The method of embodiment 142, wherein the follicular triggering period has a duration of from 1 day to 5 days.

144. The method of embodiment 143, wherein the follicular triggering period has a duration of from 1 day to 4 days.

145. The method of embodiment 144, wherein the follicular triggering period has a duration of from 1 day to 3 days.

146. The method of embodiment 145, wherein the follicular triggering period has a duration of from 2 days to 8 days.

147. The method of embodiment 146, wherein the follicular triggering period has a duration of from 2 days to 7 days.

148. The method of embodiment 147, wherein the follicular triggering period has a duration of from 2 days to 6 days.

149. The method of embodiment 148, wherein the follicular triggering period has a duration of from 2 days to 5 days.

150. The method of embodiment 149, wherein the follicular triggering period has a duration of from 2 days to 4 days.

151. The method of embodiment 130 or 131, wherein the follicular triggering period has a duration of from 3 days to 8 days.

152. The method of embodiment 151, wherein the follicular triggering period has a duration of from 3 days to 7 days.

153. The method of embodiment 152, wherein the follicular triggering period has a duration of from 3 days to 6 days.

154. The method of embodiment 153, wherein the follicular triggering period has a duration of from 3 days to 5 days.

155. The method of any one of embodiments 130-154, wherein the one or more follicular triggering agents comprise FSH, clomiphene citrate, and/or hCG.

156. The method of embodiment 155, wherein the one or more follicular triggering agents comprise FSH.

157. The method of embodiment 156, wherein the FSH is administered to the subject in one or more doses per day.

158. The method of embodiment 157, wherein the FSH is administered to the subject once daily.

159. The method of any one of embodiments 156-158, wherein the FSH is administered to the subject in an amount of from about 100 IU to about 1,000 IU per day.

160. The method of embodiment 159, wherein the FSH is administered to the subject in an amount of from about 200 IU to about 800 IU per day.

161. The method of embodiment 160, wherein the FSH is administered to the subject in an amount of from about 300 IU to about 700 IU per day.

162. The method of embodiment 161, wherein the FSH is administered to the subject in an amount of from about 300 IU to about 600 IU per day, from about 300 IU to about 500 IU per day, or from about 300 IU to about 400 IU per day.

163. The method of any one of embodiments 156-162, wherein the duration of FSH administration is equal to the duration of the follicular triggering period.

164. The method of any one of embodiments 156-162, wherein the duration of FSH administration is less than the duration of the follicular triggering period.

165. The method of embodiment 164, wherein the duration of FSH administration is 1, 2, 3, 4, or 5 days during the follicular triggering period, optionally wherein the FSH is administered to the subject in an amount of about 200 IU per day for 1, 2, 3, 4, or 5 days during the follicular triggering period, optionally wherein the FSH is administered to the subject in an amount of about 200 IU per day for 3 days during the follicular triggering period.

166. The method of any one of embodiments 155-165, wherein the one or more follicular triggering agents comprise clomiphene citrate.

167. The method of embodiment 166, wherein the clomiphene citrate is administered to the subject in one or more doses per day.

168. The method of embodiment 167, wherein the clomiphene citrate is administered to the subject once daily.

169. The method of any one of embodiments 166-168, wherein the clomiphene citrate is administered to the subject in an amount of from about 50 mg to about 100 mg per day.

170. The method of embodiment 169, wherein the clomiphene citrate is administered to the subject in an amount of about 50 mg per day.

171. The method of any one of embodiments 166-170, wherein the duration of clomiphene citrate administration is equal to the duration of the follicular triggering period.

172. The method of any one of embodiments 166-170, wherein the duration of clomiphene citrate administration is less than the duration of the follicular triggering period.

173. The method of embodiment 172, wherein the duration of clomiphene citrate administration is 1, 2, 3, 4, or 5 days during the follicular triggering period.

174. The method of any one of embodiments 155-173, wherein the one or more follicular triggering agents comprise hCG.

175. The method of embodiment 174, wherein the hCG is administered to the subject in one or more doses per day.

176. The method of embodiment 175, wherein the hCG is administered to the subject in 1, 2, or 3 doses during the follicular triggering period.

177. The method of any one of embodiments 174-176, wherein the hCG is administered to the subject in an amount of from about 200 μg to about 700 μg per dose.

178. The method of embodiment 177, wherein the hCG is administered to the subject in an amount of from about 200 μg to about 500 μg per dose, from about 300 μg to about 600 μg per dose, from about 400 μg to about 700 μg per dose, from about 200 μg to about 300 μg per dose, from about 300 μg to about 400 μg per dose, from about 400 μg to about 500 μg per dose, from about 500 μg to about 600 μg per dose, or from about 600 μg to about 700 μg per dose.

179. The method of embodiment 178, wherein the hCG is administered to the subject in an amount of about 500 μg per dose.

180. The method of any one of embodiments 174-176, wherein the hCG is administered to the subject in an amount of from about 2,500 IU to about 10,000 IU per dose.

181. The method of any one of embodiments 130-180, wherein the subject is one that has completed oral contraceptive treatment within 28 days of commencement of the follicular triggering period.

182. The method of embodiment 181, wherein the follicular triggering period commences at least 5 days after cessation of the contraceptive treatment.

183. The method of any one of embodiments 130-180, wherein the subject has not undergone oral contraceptive treatment within 28 days of commencement of the follicular triggering period.

184. The method of embodiment 183, wherein the follicular triggering period commences on day 2 of the subject's menstrual cycle.

185. The method of any one of embodiments 181-184, wherein the contraceptive treatment comprises administration to the subject of a GnRH agonist.

186. The method of any one of embodiments 130-185, wherein the subject has been determined to exhibit a follicle size of from about 6 mm to about 8 mm prior to commencement of the follicular triggering period.

187. The method of any one of embodiments 130-185, wherein the subject has been determined to exhibit a follicle size of from about 6 mm to about 8 mm prior to administration of a final follicular triggering agent.

188. The method of any one of embodiments 130-187, wherein a biological sample isolated from the subject prior to retrieval of the one or more oocytes has been determined to have an AMH concentration of from about 1 ng/mL to about 6 ng/mL.

189. The method of embodiment 188, wherein the biological sample has been determined to have an AMH concentration of from about 2 ng/mL to about 5 ng/mL.

190. The method of embodiment 189, wherein the biological sample has been determined to have an AMH concentration of from about 2.5 ng/mL to about 3.0 ng/mL.

191. The method of any one of embodiments 130-187, wherein a biological sample isolated from the subject prior to retrieval of the one or more oocytes has been determined to have an AMH concentration of at least 1 ng/mL.

192. The method of any one of embodiments 130-187, wherein a biological sample isolated from the subject prior to retrieval of the one or more oocytes has been determined to have an AMH concentration of no greater than 6 ng/mL.

193. The method of any one of embodiments 188-192, wherein the biological sample is a blood sample.

194. The method of any one of embodiments 130-193, wherein the subject is from 18 years old to 48 years old at the time of retrieval of the one or more oocytes.

195. The method of embodiment 194, wherein the subject is from 20 years old to 45 years old at the time of retrieval of the one or more oocytes.

196. The method of embodiment 194, wherein the subject is less than 35 years old at the time of retrieval of the one or more oocytes.

197. The method of embodiment 194, wherein the subject is greater than 35 years old at the time of retrieval of the one or more oocytes.

198. The method of any one of embodiments 130-197, wherein prior to retrieval of the one or more oocytes from the subject, the subject has been determined to exhibit a follicle size of from about 6 mm to about 14 mm.

199. The method of embodiment 198, wherein prior to retrieval of the one or more oocytes from the subject, the subject has been determined to exhibit a follicle size of from about 8 mm to about 12 mm.

200. The method of embodiment 199, wherein prior to retrieval of the one or more oocytes from the subject, the subject has been determined to exhibit a follicle size of from about 8 mm to about 9 mm.

201. The method of any one of embodiments 130-197, wherein prior to retrieval of the one or more oocytes from the subject, the subject has been determined to exhibit a follicle size of no greater than 14 mm.

202. The method of any one of embodiments 198-201, wherein the follicle size has been assessed by way of ultrasound image analysis.

203. The method of any one of embodiments 130-202, wherein a total of 20 oocytes or less are retrieved from the subject.

204. The method of embodiment 203, wherein 15 oocytes or less are retrieved from the subject.

205. The method of embodiment 204, wherein 10 oocytes or less are retrieved from the subject.

206. The method of embodiment 205, wherein 9 oocytes or less are retrieved from the subject.

207. The method of embodiment 206, wherein 8 oocytes or less are retrieved from the subject.

208. The method of embodiment 207, wherein 7 oocytes or less are retrieved from the subject.

209. The method of embodiment 208, wherein 6 oocytes or less are retrieved from the subject.

210. The method of embodiment 209, wherein 5 oocytes or less are retrieved from the subject.

211. The method of any one of embodiments 130-210, wherein a plurality of oocytes are retrieved from the subject.

212. The method of embodiment 211, wherein from 10% to 100% of the oocytes retrieved from the subject are GV-stage or MI-stage oocytes.

213. The method of embodiment 212, wherein from 20% to 100% of the oocytes retrieved from the subject are GV-stage or MI-stage oocytes.

214. The method of embodiment 213, wherein from 30% to 100% of the oocytes retrieved from the subject are GV-stage or MI-stage oocytes.

215. The method of embodiment 214, wherein from 40% to 100% of the oocytes retrieved from the subject are GV-stage or MI-stage oocytes.

216. The method of embodiment 215, wherein from 50% to 100% of the oocytes retrieved from the subject are GV-stage or MI-stage oocytes.

217. The method of embodiment 216, wherein from 60% to 100% of the oocytes retrieved from the subject are GV-stage or MI-stage oocytes.

218. The method of embodiment 217, wherein from 70% to 100% of the oocytes retrieved from the subject are GV-stage or MI-stage oocytes

219. The method of embodiment 218, wherein from 80% to 100% of the oocytes retrieved from the subject are GV-stage or MI-stage oocytes.

220. The method of embodiment 219, wherein from 90% to 100% of the oocytes retrieved from the subject are GV-stage or MI-stage oocytes.

221. The method of embodiment 220, wherein 100% of the oocytes retrieved from the subject are GV-stage or MI-stage oocytes.

222. The method of any one of embodiments 130-221, wherein the population of ovarian support cells comprises ovarian granulosa cells and/or ovarian stroma cells, optionally wherein the ovarian granulosa cells are FOXL2-positive and/or wherein the ovarian stroma cells are NR2F2-positive.

223. The method of any one of embodiments 130-222, wherein the population of ovarian support cells comprises from about 50,000 to about 100,000 ovarian support cells.

224. The method of any one of embodiments 130-222, wherein the population of ovarian support cells comprises from about 50,000 to about 60,000 ovarian support cells, from about 60,000 to about 70,000 ovarian support cells, from about 70,000 to about 80,000 ovarian support cells, from about 80,000 to about 90,000 ovarian support cells, or from about 90,000 to about 100,000 ovarian support cells.

225. The method of any one of embodiments 130-222, wherein the population of ovarian support cells comprises about 50,000 ovarian support cells, about 55,000 ovarian support cells, about 60,000 ovarian support cells, about 65,000 ovarian support cells, about 70,000 ovarian support cells, about 75,000 ovarian support cells, about 80,000 ovarian support cells, about 85,000 ovarian support cells, about 90,000 ovarian support cells, about 95,000 ovarian support cells, or about 100,000 ovarian support cells.

226. The method of any one of embodiments 222-225, wherein the ovarian granulosa cells comprise steroidogenic granulosa cells

227. The method of embodiment 226, wherein the steroidogenic granulosa cells produce estradiol.

228. The method of any one of embodiments 130-227, wherein the ovarian support cells are obtained by differentiation of a population of iPSCs.

229. The method of embodiment 228, wherein the ovarian support cells are obtained by modifying the iPSCs to express one or more transcription factors selected from FOXL2, NR5A1, GATA4, RUNX1, and RUNX2.

230. The method of embodiment 229, wherein the ovarian support cells are obtained by modifying the iPSCs to express two or more of FOXL2, NR5A1, GATA4, RUNX1, and RUNX2.

231. The method of embodiment 230, wherein the ovarian support cells are obtained by modifying the iPSCs to express three or more of FOXL2, NR5A1, GATA4, RUNX1, and RUNX2.

232. The method of embodiment 231, wherein the ovarian support cells are obtained by modifying the iPSCs to express four or more of FOXL2, NR5A1, GATA4, RUNX1, and RUNX2.

233. The method of embodiment 232, wherein the ovarian support cells are obtained by modifying the iPSCs to express all five of FOXL2, NR5A1, GATA4, RUNX1, and RUNX2.

234. The method of any one of embodiments 130-233, wherein the ovarian support cells are cryopreserved and thawed prior to the co-culturing with the one or more oocytes.

235. The method of embodiment 234, wherein the ovarian support cells are thawed from about 24 hours to about 120 hours prior to the co-culturing with the one or more oocytes.

236. The method of embodiment 234, wherein the ovarian support cells are thawed from about 24 hours to about 48 hours, from about 48 hours to about 72 hours, from about 72 hours to about 96 hours, or from about 96 hours to about 120 hours prior to the co-culturing with the one or more oocytes.

237. The method of embodiment 234, wherein the ovarian support cells are thawed from about 24 hours to about 36 hours, from about 30 hours to about 40 hours, from about 36 hours to about 48 hours, from about 48 hours to about 56 hours, from about 56 hours to about 72 hours, from about 72 hours to about 84 hours, from about 80 hours to about 96 hours, from about 90 hours to about 100 hours, from about 96 hours to about 108 hours, or from about 108 hours to about 120 hours prior to the co-culturing with the one or more oocytes.

238. The method of any one of embodiments 130-237, wherein the one or more oocytes are co-cultured with the population of ovarian support cells for from about 12 hours to about 120 hours.

239. The method of any one of embodiments 130-237, wherein the one or more oocytes are co-cultured with the population of ovarian support cells for from about 12 hours to about 24 hours, from about 12 hours to about 36 hours, from about 24 hours to about 48 hours, from about 36 hours to about 60 hours, from about 54 hours to about 72 hours, from about 68 hours to about 96 hours, or from about 96 hours to about 120 hours.

240. The method of any one of embodiments 130-237, wherein the one or more oocytes are co-cultured with the population of ovarian support cells for about 12 hours, about 14 hours, about 16 hours, about 18 hours, about 20 hours, about 22 hours, about 24 hours, about 26 hours, about 28 hours, about 30 hours, about 32 hours, about 34 hours, about 36 hours, about 38 hours, about 40 hours, about 42 hours, about 44 hours, about 46 hours, about 48 hours, about 50 hours, about 52 hours, about 54 hours, about 56 hours, about 58 hours, about 60 hours, about 62 hours, about 64 hours, about 66 hours, about 68 hours, about 70 hours, about 72 hours, about 74 hours, about 76 hours, about 78 hours, about 80 hours, about 82 hours, about 84 hours, about 86 hours, about 88 hours, about 90 hours, about 92 hours, about 94 hours, about 96 hours, about 98 hours, about 100 hours, about 102 hours, about 104 hours, about 106 hours, about 108 hours, about 110 hours, about 112 hours, about 114 hours, about 116 hours, about 118 hours, or about 120 hours.

241. The method of any one of embodiments 130-240, wherein the co-culturing is conducted in an adherent co-culture system.

242. The method of any one of embodiments 130-240, wherein the co-culturing is conducted in a suspension co-culture system.

243. The method of any one of embodiments 130-242, wherein prior to and/or after the co-culturing, the one or more oocytes are evaluated for a parameter selected from the group consisting of total oocyte score, GV-stage to MII-stage oocyte maturation rate, GV-stage to MI-stage oocyte maturation rate, MI-stage to MII-stage oocyte maturation rate, average oocyte shape, average oocyte size, average ooplasm quality, average perivitelline space (PVS) quality, average zona pellucida (ZP) quality, and average polar body quality.

244. The method of any one of embodiments 130-243, wherein the one or more oocytes are denuded following the co-culturing.

245. The method of any one of embodiments 130-244, the method further comprising isolating one or more MII-stage oocytes from the mixture produced by co-culturing the one or more oocytes retrieved from the subject with the population of ovarian support cells.

246. The method of embodiment 245, wherein the subject is undergoing an autologous ART procedure, and wherein the method further comprises contacting each of the one or more MII-stage oocytes with a mature sperm cell.

247. The method of embodiment 246, wherein the one or more MII-stage oocytes are cryopreserved and thawed prior to the contacting.

248. The method of embodiment 246, wherein the one or more MII-stage oocytes are not cryopreserved and thawed prior to the contacting.

249. The method of any one of embodiments 246-248, wherein the contacting comprises IVF of the one or more MII-stage oocytes.

250. The method of any one of embodiments 246-248, wherein the contacting comprises ICSI into the one or more MII-stage oocytes.

251. The method of any one of embodiments 246-250, wherein the contacting results in formation of an embryo.

252. The method of embodiment 251, wherein the embryo is transferred to the uterus of the subject.

253. The method of embodiment 252, wherein the embryo is transferred to the uterus of the subject about 3 days following the contacting of the one or more MII-stage oocytes with a mature sperm cell.

254. The method of embodiment 252, wherein the embryo is transferred to the uterus of the subject about 5 days following the contacting of the one or more MII-stage oocytes with a mature sperm cell.

255. The method of embodiment 252, wherein the embryo transferred to the uterus of the subject is a blastocyst-stage embryo.

256. An ex vivo composition comprising a population of ovarian support cells and one or more diluents or excipients, optionally wherein the population comprises from about 10,000 to about 100,000 ovarian support cells

257. The composition of embodiment 256, wherein the population of ovarian support cells comprises from about 50,000 to about 100,000 ovarian support cells.

258. The composition of embodiment 256, wherein the population of ovarian support cells comprises from about 50,000 to about 60,000 ovarian support cells, from about 60,000 to about 70,000 ovarian support cells, from about 70,000 to about 80,000 ovarian support cells, from about 80,000 to about 90,000 ovarian support cells, or from about 90,000 to about 100,000 ovarian support cells.

259. The composition of embodiment 256, wherein the population of ovarian support cells comprises about 50,000 ovarian support cells, about 55,000 ovarian support cells, about 60,000 ovarian support cells, about 65,000 ovarian support cells, about 70,000 ovarian support cells, about 75,000 ovarian support cells, about 80,000 ovarian support cells, about 85,000 ovarian support cells, about 90,000 ovarian support cells, about 95,000 ovarian support cells, or about 100,000 ovarian support cells.

260. The composition of any one of embodiments 256-259, wherein the ovarian support cells comprise steroidogenic granulosa cells.

261. The composition of embodiment 260, wherein the steroidogenic granulosa cells produce estradiol.

262. The composition of any one of embodiments 256-261, wherein the ovarian support cells are obtained by differentiation of a population of iPSCs.

263. The composition of embodiment 262, wherein the ovarian support cells are obtained by modifying the iPSCs to express one or more transcription factors selected from FOXL2, NR5A1, GATA4, RUNX1, and RUNX2.

264. The composition of embodiment 263, wherein the ovarian support cells are obtained by modifying the iPSCs to express two or more of FOXL2, NR5A1, GATA4, RUNX1, and RUNX2.

265. The composition of embodiment 264, wherein the ovarian support cells are obtained by modifying the iPSCs to express three or more of FOXL2, NR5A1, GATA4, RUNX1, and RUNX2.

266. The composition of embodiment 265, wherein the ovarian support cells are obtained by modifying the iPSCs to express four or more of FOXL2, NR5A1, GATA4, RUNX1, and RUNX2.

267. The composition of embodiment 266, wherein the ovarian support cells are obtained by modifying the iPSCs to express all five of FOXL2, NR5A1, GATA4, RUNX1, and RUNX2.

268. The composition of any one of embodiments 256-267, wherein the ovarian support cells are cryopreserved.

269. A cell culture medium comprising a population of ovarian support cells, optionally wherein the population comprises from about 10,000 to about 150,000 ovarian support cells.

270. The cell culture medium of embodiment 269, wherein the population of ovarian support cells comprises from about 50,000 to about 150,000 ovarian support cells.

271. The cell culture medium of embodiment 269, wherein the population of ovarian support cells comprises from about 50,000 to about 60,000 ovarian support cells, from about 60,000 to about 70,000 ovarian support cells, from about 70,000 to about 80,000 ovarian support cells, from about 80,000 to about 90,000 ovarian support cells, from about 90,000 to about 100,000 ovarian support cells, from about 100,000 to about 110,000 ovarian support cells, from about 110,000 to about 120,000 ovarian support cells, from about 120,000 to about 130,000 ovarian support cells, from about 130,000 to about 140,000 ovarian support cells, or from about 140,000 to about 150,000 ovarian support cells.

272. The cell culture medium of embodiment 269, wherein the population of ovarian support cells comprises about 50,000 ovarian support cells, about 55,000 ovarian support cells, about 60,000 ovarian support cells, about 65,000 ovarian support cells, about 70,000 ovarian support cells, about 75,000 ovarian support cells, about 80,000 ovarian support cells, about 85,000 ovarian support cells, about 90,000 ovarian support cells, about 95,000 ovarian support cells, about 100,000 ovarian support cells, about 105,000 ovarian support cells, about 110,000 ovarian support cells, about 115,000 ovarian support cells, about 120,000 ovarian support cells, about 125,000 ovarian support cells, about 130,000 ovarian support cells, about 135,000 ovarian support cells, about 140,000 ovarian support cells, about 145,000 ovarian support cells, or about 150,000 ovarian support cells.

273. The cell culture medium of any one of embodiments 269-272, wherein the ovarian support cells comprise steroidogenic granulosa cells.

274. The cell culture medium of embodiment 273, wherein the steroidogenic granulosa cells produce estradiol.

275. The cell culture medium of any one of embodiments 269-274, wherein the ovarian support cells are obtained by differentiation of a population of iPSCs.

276. The cell culture medium of embodiment 275, wherein the ovarian support cells are obtained by modifying the iPSCs to express one or more transcription factors selected from FOXL2, NR5A1, GATA4, RUNX1, and RUNX2.

277. The cell culture medium of embodiment 276, wherein the ovarian support cells are obtained by modifying the iPSCs to express two or more of FOXL2, NR5A1, GATA4, RUNX1, and RUNX2.

278. The cell culture medium of embodiment 277, wherein the ovarian support cells are obtained by modifying the iPSCs to express three or more of FOXL2, NR5A1, GATA4, RUNX1, and RUNX2.

279. The cell culture medium of embodiment 278, wherein the ovarian support cells are obtained by modifying the iPSCs to express four or more of FOXL2, NR5A1, GATA4, RUNX1, and RUNX2.

280. The cell culture medium of embodiment 279, wherein the ovarian support cells are obtained by modifying the iPSCs to express all five of FOXL2, NR5A1, GATA4, RUNX1, and RUNX2.

281. The cell culture medium of any one of embodiments 269-280, wherein the cell culture medium is cryopreserved

282. The composition of any one of embodiments 256-268 or the cell culture medium of any one of embodiments 269-281 for use in performing the method of any one of embodiments 1-255.

283. A kit comprising the composition of any one of embodiments 256-268 and a package insert, wherein the package insert instructs a user of the kit to co-culture the population of ovarian support cells with one or more oocytes in accordance with the method of any one of embodiments 1-255.

284. A kit comprising the cell culture medium of any one of embodiments 269-281 and a package insert, wherein the package insert instructs a user of the kit to co-culture the population of ovarian support cells with one or more oocytes in accordance with the method of any one of embodiments 1-255.

285. An apparatus for aiding in human oocyte maturation in vitro, the apparatus comprising:
a computing device, wherein the computing device comprises:
at least a processor; and
a memory communicatively connected to the at least processor, the memory containing instructions configuring the at least processor to:
   receive first biological sample data from a first biological sample relating to a user;
   assign the user to a stimulation protocol as a function of the first biological sample;
      receive second biological sample data from a second biological sample relating to the user wherein the second biological sample comprises at least an oocyte;
   receive culture data relating to the second biological sample; and assign the second biological sample a scoring metric as a function of the culture data of the second biological sample.

286. The apparatus of embodiment 285, wherein the first biological sample from the user comprises blood.

287. The apparatus of embodiment 285, wherein the stimulation protocol is assigned based on a measured hormone level.

288. The apparatus of embodiment 285, wherein the stimulation protocol further comprises a minimal stimulation protocol.

289. The apparatus of embodiment 288, wherein the minimal stimulation protocol further comprises:
selecting a first triggering agent as a function of the first biological sample; and
selecting a second triggering agent as a function of a follicle measurement.

290. The apparatus of embodiment 285, wherein culture data further comprises culturing the second biological sample comprising the at least an oocyte in a granulosa group culture.

291. The apparatus of embodiment 290, wherein the granulosa group culture comprises from about 50,000-500,000 granulosa cells.

292. The apparatus of embodiment 290, wherein the group culture further comprises a cell culture metabolite.

293. The apparatus of embodiment 285, wherein the scoring metric comprises oocyte scoring.

294. The apparatus of embodiment 285, wherein the scoring metric comprises outcome analysis.

295. A method for inducing human oocyte maturation in vitro, the method comprising:
receiving a first biological sample relating to a user;
assigning the user to a stimulation protocol as a function of the first biological sample;
receiving a second biological sample relating to the user wherein the second biological sample comprises at least an oocyte;
   culturing the second biological sample; and
assigning the second biological sample a scoring metric as a function of culturing the second biological sample.

296. The method of embodiment 295, wherein the first biological sample from the user comprises blood.

297. The method of embodiment 295, wherein the stimulation protocol is assigned based on a measured hormone level.

298. The method of embodiment 295, wherein the stimulation protocol further comprises a minimal stimulation protocol.

299. The method of embodiment 298, wherein the minimal stimulation protocol further comprises:
selecting a first triggering agent as a function of the first biological sample; and
selecting a second triggering agent as a function of a follicle measurement.

300. The method of embodiment 295, wherein culturing the second biological sample comprises culturing the at least an oocyte in a granulosa group culture.

301. The method of embodiment 300, wherein the granulosa group culture further comprises from about 50,000-500,000 granulosa cells.

302. The method of embodiment 300, wherein the group culture further comprises a cell culture metabolite.

303. The method of embodiment 295, wherein the scoring metric comprises oocyte scoring.

304. The method of embodiment 295, wherein the scoring metric comprises outcome analysis.

305. An apparatus for aiding in oocyte rescue in vitro post stimulation, the apparatus comprising:
a computing device, wherein the computing device comprises:
at least a processor; and
   a memory communicatively connected to the at least processor, the memory containing instructions configuring the at least processor to:
receive biological sample data from a biological sample relating to a user, wherein the biological sample comprises at least an oocyte;
determine a maturity level of the at least an oocyte as a function of the biological sample data;
assign the at least an oocyte to a culture protocol as a function of the maturity level;
   receive culture data relating to the at least an oocyte as a function of the culture protocol; and
calculate a scoring metric as a function of the culture data.

306. The apparatus of embodiment 305, wherein the at least an oocyte comprises a cumulus-oocyte-complex.

307. The apparatus of embodiment 305, wherein determining the maturity level of the at least an oocyte further comprises denuding the oocyte.

308. The apparatus of embodiment 305, wherein determining the maturity level of the at least oocyte furth comprises using a machine learning process.

309. The apparatus of embodiment 305, wherein assigning the culture protocol further comprises selecting a cell culture metabolite as a function of the maturity level.

310. The apparatus of embodiment 305, wherein assigning the culture protocol further comprises selecting a cell culture medium as a function of the maturity level.

311. The apparatus of embodiment 305, wherein the culture protocol further comprises culturing the at least an oocyte with a granulosa co-culture containing granulosa cells sourced from human induced pluripotent stem cells (hiPSCs).

312. The apparatus of embodiment 311, wherein the culture protocol further comprises culturing the at least oocyte with a granulosa co-culture for 24 hours.

313. The apparatus of embodiment 305, wherein the scoring metric comprises omics-based analysis.

314. The apparatus of embodiment 313, wherein omics-based analysis includes genomics.

315. A method for oocyte rescue in vitro post stimulation, the method comprising:
receiving a biological sample relating to a user, comprising at least an oocyte;
   determining a maturity level of the at least an oocyte;
assigning the at least an oocyte to a culture protocol as a function of the maturity level;
culturing the at least an oocyte as a function of the culture protocol; and calculating a scoring metric as a function of the cultured oocyte.

316. The method of embodiment 315, wherein the at least an oocyte comprises a cumulus-oocyte-complex.

317. The method of embodiment 315, wherein determining the maturity level of the at least an oocyte further comprises denuding the oocyte.

318. The method of embodiment 315, wherein assigning the culture protocol further comprises selecting a cell culture metabolite as a function of the maturity level.

319. The method of embodiment 315, wherein assigning the culture protocol further comprises selecting a cell culture medium as a function of the maturity level.

320. The method of embodiment 315, wherein the culture protocol further comprises culturing the at least an oocyte with a granulosa co-culture containing granulosa cells sourced from human induced pluripotent stem cells (hiPSCs).

321. The method of embodiment 315, wherein the culture protocol further comprises culturing the at least oocyte with a granulosa co-culture for 24 hours.

322. The method of embodiment 315, wherein the culture protocol further comprises flash freezing the culture media.

323. The method of embodiment 315, wherein the scoring metric comprises omics-based analysis.

324. The method of embodiment 323, wherein omics-based analysis includes genomics.

325. A method of producing a mature oocyte for use in an ART procedure, the method comprising:
(a) administering to a human subject one or more follicular triggering agents comprising FSH during a follicular triggering period;
(b) retrieving one or more oocytes from the subject following the follicular triggering period; and
(c) culturing the one or more oocytes with a population of ovarian support cells, thereby producing one or more mature oocytes,
wherein the duration of FSH administration is 1, 2, 3, 4, or 5 days during the follicular triggering period, optionally wherein the FSH is administered to the subject in an amount of about 200 IU per day for 1, 2, 3, 4, or 5 days during the follicular triggering period, optionally wherein the FSH is administered to the subject in an amount of about 200 IU per day for 3 days during the follicular triggering period.

326. A method of producing a mature oocyte for use in an ART procedure, the method comprising:
(a) administering to a human subject one or more follicular triggering agents comprising FSH during a follicular triggering period;
(b) retrieving one or more oocytes from the subject following the follicular triggering period; and
(c) culturing the one or more oocytes with a population of ovarian support cells, thereby producing one or more mature oocytes,
wherein the duration of FSH administration is 1, 2, 3, 4, or 5 days during the follicular triggering period, optionally wherein the FSH is administered to the subject in an amount of about 200 IU per day for 1, 2, 3, 4, or 5 days during the follicular triggering period, optionally wherein the FSH is administered to the subject in an amount of about 200 IU per day for 3 days during the follicular triggering period, and
wherein the subject has been determined to exhibit a follicle size of from about 6 mm to about 8 mm prior to commencement of the follicular triggering period.

327. A method of producing a mature oocyte for use in an ART procedure, the method comprising:
(a) retrieving one or more oocytes from the subject; and
(b) culturing the one or more oocytes with a population of ovarian support cells, thereby producing one or more mature oocytes,
wherein the subject is not administered a follicular triggering agent prior to the retrieving of the one or more oocytes.

### Other Embodiments

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations following, in general, the principles and including such departures from the invention that come within known or customary practice within the art to which the invention pertains and may be applied to the essential features hereinbefore set forth, and follows in the scope of the claims.

Other embodiments are within the claims.

1. A method of inducing in vitro maturation of one or more oocytes that have previously been retrieved from a human subject, the method comprising co-culturing the one or more oocytes with a population of ovarian support cells.

2. The method of claim 1, wherein the subject is not administered a follicular triggering agent prior to retrieval of the one or more oocytes from the subject.

3. The method of claim 1, wherein prior to retrieval of the one or more oocytes from the subject, the subject is administered one or more follicular triggering agents during a follicular triggering period.

4. The method of claim 3, wherein the follicular triggering period has a duration of no greater than 8 days.

5. The method of claim 3, wherein the one or more follicular triggering agents comprise follicle stimulating hormone (FSH), clomiphene citrate, and/or human chorionic gonadotropin (hCG).

6. The method of claim 5, wherein the one or more follicular triggering agents comprise FSH, wherein:
(a) the FSH is administered to the subject in one or more doses per day;
(b) the FSH is administered to the subject in an amount of from about 100 international units (IU) to about 1,000 IU per day; and/or
(c) the duration of FSH administration is equal to or less than the duration of the follicular triggering period.

7. The method of claim 6, wherein the duration of FSH administration is less than the duration of the follicular triggering period, wherein:
(a) the duration of FSH administration is 1, 2, 3, 4, or 5 days during the follicular triggering period;
(b) the FSH is administered to the subject in an amount of about 200 IU per day for 1, 2, 3, 4, or 5 days during the follicular triggering period; or
(c) the FSH is administered to the subject in an amount of about 200 IU per day for 3 days during the follicular triggering period.

8. The method of claim 5, wherein the one or more follicular triggering agents comprise clomiphene citrate, wherein:
(a) the clomiphene citrate is administered to the subject in one or more doses per day;
(b) the clomiphene citrate is administered to the subject in an amount of from about 50 mg to about 100 mg per day; and/or
(c) the duration of clomiphene citrate administration is equal to or less than the duration of the follicular triggering period.

9. The method of claim 8, wherein the duration of clomiphene citrate administration is less than the duration of the follicular triggering period, wherein the duration of clomiphene citrate administration is 1, 2, 3, 4, or 5 days during the follicular triggering period.

10. The method of claim 5, wherein the one or more follicular triggering agents comprise hCG, wherein:
(a) the hCG is administered to the subject in one or more doses per day;
(b) the hCG is administered to the subject in 1, 2, or 3 doses during the follicular triggering period; and/or
(c) the hCG is administered to the subject in an amount of from about 200 μg to about 700 μg per dose or from about 2,500 IU to about 10,000 IU per dose.

11. The method of claim 3, wherein the subject is one that has completed or has not undergone oral contraceptive treatment within 28 days of commencement of the follicular triggering period.

12. The method of claim 11, wherein the follicular triggering period commences at least 5 days after cessation of the contraceptive treatment or on day 2 of the subject's menstrual cycle.

13. The method of claim 3, wherein the subject has been determined to exhibit a follicle size of from about 6 mm to about 8 mm prior to commencement of the follicular triggering period or prior to administration of a final follicular triggering agent.

14. The method of claim 1, wherein a biological sample isolated from the subject prior to retrieval of the one or more oocytes has been determined to have an anti-Mullerian hormone (AMH) concentration of from about 0.1 ng/mL to about 1 ng/mL, or from about 1 ng/mL to about 6 ng/mL.

15. The method of claim 1, wherein:
(a) the subject is from 18 years old to 48 years old at the time of retrieval of the one or more oocytes;
(b) prior to retrieval of the one or more oocytes from the subject, the subject has been determined to exhibit a follicle size of from about 6 mm to about 14 mm; and/or
(c) a plurality of oocytes are retrieved from the subject.

16. The method of claim 1, wherein a plurality of oocytes are retrieved from the subject, wherein from 10% to 100% of the oocytes retrieved from the subject are germinal vesicle (GV)-stage or meiosis I (MI)-stage oocytes.

17. The method of claim 1, wherein the population of ovarian support cells comprises:
(a) ovarian granulosa cells and/or ovarian stroma cells;
(b) forkhead box protein L2 (FOXL2)-positive ovarian granulosa cells and/or nuclear receptor subfamily 2 group F member 2 (NR2F2)-positive ovarian stroma cells; and/or
(c) about 50,000 to about 500,000 ovarian support cells.

18. The method of claim 17, wherein the ovarian support cells comprise steroidogenic granulosa cells.

19. The method of claim 1, wherein the ovarian support cells are obtained by differentiation of a population of induced pluripotent stem cells (iPSCs).

20. The method of claim 1, wherein the ovarian support cells heterologously express:
(a) one or more of transcription factors FOXL2, NR5A1, GATA4, RUNX1, and RUNX2;
(b) two or more of transcription factors FOXL2, NR5A1, GATA4, RUNX1, and RUNX2;
(c) three or more of transcription factors FOXL2, NR5A1, GATA4, RUNX1, and RUNX2;
(d) four or more of transcription factors FOXL2, NR5A1, GATA4, RUNX1, and RUNX2; or
(e) all five of transcription factors FOXL2, NR5A1, GATA4, RUNX1, and RUNX2.

21. The method of claim 1, wherein:
(a) the one or more oocytes are denuded following the co-culturing; and/or
(b) the method further comprises isolating one or more meiosis II (MII)-stage oocytes from the mixture produced by co-culturing the one or more oocytes retrieved from the subject with the population of ovarian support cells.

22. The method of claim 21, wherein the method further comprises isolating one or more MII-stage oocytes from the mixture, wherein the subject is undergoing an autologous ART procedure, and wherein the method further comprises contacting each of the one or more MII-stage oocytes with a mature sperm cell, wherein the contacting comprises in vitro fertilization (IVF) of the one or more MII-stage oocytes or intracytoplasmic sperm injection (ICSI) into the one or more MII-stage oocytes, and/or wherein the contacting results in formation of an embryo, optionally wherein the embryo is a blastocyst embryo and/or is transferred to the uterus of the subject.

23. An ex vivo composition comprising a population of ovarian support cells, one or more diluents or excipients, and one or more GV-stage or MI-stage stage oocytes, optionally wherein the population comprises from about 10,000 to about 100,000 ovarian support cells.

24. The ex vivo composition of claim 23, wherein the population of ovarian support cells heterologously express:
(a) one or more of transcription factors FOXL2, NR5A1, GATA4, RUNX1, and RUNX2;
(b) two or more of transcription factors FOXL2, NR5A1, GATA4, RUNX1, and RUNX2;
(c) three or more of transcription factors FOXL2, NR5A1, GATA4, RUNX1, and RUNX2;
(d) four or more of transcription factors FOXL2, NR5A1, GATA4, RUNX1, and RUNX2; or
(e) all five of transcription factors FOXL2, NR5A1, GATA4, RUNX1, and RUNX2.

25. The ex vivo composition of claim 23, wherein the population of ovarian support cells comprise:
(a) ovarian granulosa cells and/or ovarian stroma cells, optionally wherein the ovarian granulosa cells are steroidogenic granulosa cells;
(b) FOXL2-positive ovarian granulosa cells and/or NR2F2-positive ovarian stroma cells.

26. An ex vivo cell culture medium comprising a population of ovarian support cells and one or more GV-stage or MI-stage oocytes, optionally wherein the population comprises from about 10,000 to about 150,000 ovarian support cells.

27. The cell culture medium of claim 26, wherein the ovarian support cells heterologously express:
(a) one or more of transcription factors FOXL2, NR5A1, GATA4, RUNX1, and RUNX2;
(b) two or more of transcription factors FOXL2, NR5A1, GATA4, RUNX1, and RUNX2;
(c) three or more of transcription factors FOXL2, NR5A1, GATA4, RUNX1, and RUNX2;
(d) four or more of transcription factors FOXL2, NR5A1, GATA4, RUNX1, and RUNX2; or
(e) all five of transcription factors FOXL2, NR5A1, GATA4, RUNX1, and RUNX2.

28. The cell culture medium of claim 26, wherein the population of ovarian support cells comprise:
(a) ovarian granulosa cells and/or ovarian stroma cells, optionally wherein the ovarian granulosa cells are steroidogenic granulosa cells; and/or
(b) FOXL2-positive ovarian granulosa cells and/or NR2F2-positive ovarian stroma cells.

29. The cell culture medium of claim 26, wherein the cell culture medium comprises:
(a) human serum albumin;
(b) FSH;
(c) hCG; and/or
(d) androstenedione.

30. The cell culture medium of claim 29, wherein the cell culture medium comprises:
(a) human serum albumin at a concentration of 5 mg/mL to 15 mg/mL;
(b) FSH at a concentration of 70 mIU/mL to 80 mIU/mL;
(c) hCG, at a concentration of 95 mIU/mL to 105 mIU/mL; and/or
(d) androstenedione at a concentration of 495 ng/mL to 505 ng/mL.
